(19) United States
(12) Patent Application Publication
(10) Pub. No.: US 2024/0092783 A1
(43) Pub. Date: Mar. 21, 2024

(54) HETEROAROMATIC COMPOUNDS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Christian EHRENREICH, Darmstadt (DE)

(21) Appl. No.: 18/282,239

(22) PCT Filed: Mar. 15, 2022
(86) PCT No.: PCT/EP2022/056584

§ 371 Date: Sep. 15, 2023

(30) Foreign Application Priority Data
Mar. 18, 2021 (EP) 21163561.0

(51) Int. Cl. C07D 471/14; C07D 403/04; C07D 487/14; C07F 15/00; H10K 50/11; H10K 50/15; H10K 50/18; H10K 85/60

(52) U.S. Cl. CPC C07D 471/14; C07D 403/04; C07D 487/14; C07F 15/0033; H10K 50/11; H10K 50/15; H10K 50/181; H10K 85/6572

(57) ABSTRACT

The present invention relates to heteroaromatic compounds suitable for use in electronic devices, and to electronic devices, in particular organic electroluminescent devices, containing said compounds.

The present invention relates to heteroaromatic compounds suitable for use in electronic devices, especially in organic electroluminescent devices, and to electronic devices, especially organic electroluminescent devices, comprising these materials.

Emitting materials used in organic electroluminescent devices are frequently phosphorescent organometallic complexes. For quantum-mechanical reasons, up to four times the energy efficiency and power efficiency is possible using organometallic compounds as phosphorescent emitters. In electroluminescent devices, especially also in electroluminescent devices that exhibit triplet emission (phosphorescence), there is generally still a need for improvement. The properties of phosphorescent electroluminescent devices are determined not only by the triplet emitters used. More particularly, the other materials used, such as matrix materials, are also of particular significance here. Improvements in these materials can thus also lead to distinct improvements in the properties of the electroluminescent devices.

The publication Tetrahedron Letters 41 (2000) 5857-5860 describes the synthesis of quinazoline derivatives. However, this document does not contain any pointers to use in an electronic device.

In general terms, in the case of these materials, for example for use as matrix materials, there is still a need for improvement, particularly in relation to the lifetime, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage. Particularly the properties of the matrix materials too have a major influence on the lifetime and efficiency of the organic electroluminescent device.

A further problem addressed by the present invention can be considered that of providing compounds suitable for use in phosphorescent or fluorescent electroluminescent devices, especially as a matrix material. A particular problem addressed by the present invention is that of providing matrix materials that are suitable for red- and yellow-phosphorescing electroluminescent devices, especially for red- and green-phosphorescing electroluminescent devices, and if appropriate also for blue-phosphorescing electroluminescent devices.

In addition, the compounds, especially when they are used as matrix materials, as electron transport materials or as hole blocker materials in organic electroluminescent devices, should lead to devices having excellent color purity.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, particular compounds described in detail below solve this problem and are of good suitability for use in electroluminescent devices and lead to improvements in the organic electroluminescent device, especially in relation to lifetime, color purity, efficiency and operating voltage. The present invention therefore provides these compounds and electronic devices, especially organic electroluminescent devices, comprising such compounds.

The present invention provides a compound comprising at least one structure having at least three mutually fused structural elements of the formulae (A), (B) and (C), preferably a compound consisting of three mutually fused structural elements of the formulae (A), (B) and (C):

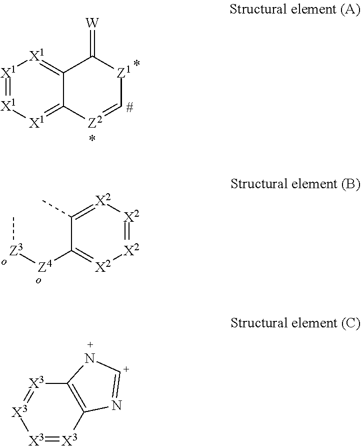

where structural element (A) is fused to structural element (B) and structural element (B) is fused to structural element (C); where structural element (B) binds to structural element (A) via the bonds shown by dotted lines, where there is one bond via the binding site marked # and one bond via a binding site marked *; where structural element (B) is fused to structural element (C) via the atoms marked o and +, and the respectively marked atoms are shared by structural elements (B) and (C), and
the symbols and indices used are as follows:
W is O or S, preferably O;
Z¹ is NAr, or N if structural element (A) is fused to structural element (B) via Z¹;
Z² is X, or C if structural element (A) is fused to structural element (B) via Z²
Z³, Z⁴ is N or C, where one of the Z³, Z⁴ radicals is N and one of the Z³, Z⁴ radicals is C;
X is N or CR, preferably N;
X¹ is the same or different at each instance and is N or CR¹, preferably CR¹, with the proviso that not more than two of the X¹ groups in one cycle are N;
X² is the same or different at each instance and is N or CR², with the proviso that not more than two of the X² groups in one cycle are N;
X³ is the same or different at each instance and is N or CR³, preferably CR³, with the proviso that not more than two of the X³ groups in one cycle are N;
Ar is the same or different at each instance and is an aromatic or heteroaromatic ring system which has 5 to 40 aromatic ring atoms and has R radicals, preferably an aryl group which has 6 to 30 aromatic ring atoms or a heteroaryl group which has 5 to 14 aromatic ring atoms and has R radicals;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for two R radicals or one R radical together with a further radical, preferably an R¹, R², R³ group, to form an aliphatic, heteroaliphatic, aromatic or heteroaromatic ring system, preferably an aliphatic, heteroaliphatic or heteroaromatic ring system; more preferably, the R radical does not form any such ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals or one R¹ radical together with a further radical, preferably an R, R², R³ group, to form an aromatic, heteroaromatic, aliphatic or heteroaliphatic ring system, preferably an aliphatic, heteroaliphatic or heteroaromatic ring system; more preferably, the R¹ radicals do not form any such ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, preferably an R, R¹, R³ group, to form an aromatic, heteroaromatic, aliphatic or heteroaliphatic ring system, preferably an aliphatic, heteroaliphatic or heteroaromatic ring system; more preferably, the R² radicals do not form any such ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, preferably an R, R¹, R² group, to form an aromatic, heteroaromatic, aliphatic or heteroaliphatic ring system, preferably an aliphatic, heteroaliphatic or heteroaromatic ring system; more preferably, the R³ radicals do not form any such ring system;
Ar′ is the same or different at each instance and is an aromatic or heteroaromatic ring system which has 5 to 40 aromatic ring atoms and has R⁴ radicals, preferably an aryl group which has 6 to 30 aromatic ring atoms or a heteroaryl group which has 5 to 14 aromatic ring atoms and has R⁴ radicals;
R⁴ is the same or different at each instance and is H, D, F, Cl, Br, I, R⁵C≡C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system, preferably an aliphatic ring system; more preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
   where at least one of the R, R¹, R², R³ radicals is selected from the group consisting of a heteroaromatic ring system which has 6 to 60 aromatic ring atoms and has R⁴ radicals, an aromatic ring system which has 10 to 60 aromatic ring atoms and has R⁴ radicals, an aryloxy group having 10 to 60 aromatic ring atoms or heteroaryloxy group having 6 to 40 aromatic ring atoms, each of which has R⁴ radicals, a diarylamino group having 6 to 60 aromatic ring atoms in the respective aromatic radical, an arylheteroarylamino group having 6 to 60 aromatic ring atoms in the respective aromatic or heteroaromatic radical, and a diheteroarylamino group having 6 to 60 aromatic ring atoms in the respective heteroaromatic radical, where the diarylamino, arylheteroarylamino, diheteroarylamino group has R⁴ radicals.

The R, R¹, R², R³ groups have R⁴ radicals, where the R⁴ radical may be H. If R⁴ is not H, the R⁴ radical is a substituent, and so the R, R¹, R², R³ groups may be substituted by R⁴ radicals. This clarification applies correspondingly to the further groups and radicals.

Two R radicals or one R radical together with a further radical, preferably an R¹, R², R³ group, may together form an aromatic, heteroaromatic, aliphatic or heteroaliphatic ring system. If a ring system is formed, this is preferably formed by two R¹ radicals, giving rise to a fused ring system. This is also applicable to the further radicals, especially to two R¹, R², R³ radicals.

In a further preferred embodiment, it may be the case that the compounds of the invention include a structure of the formulae (I-1) to (I-7), where the compounds of the invention may more preferably be selected from the compounds of the formulae (I-1) to (I-7)

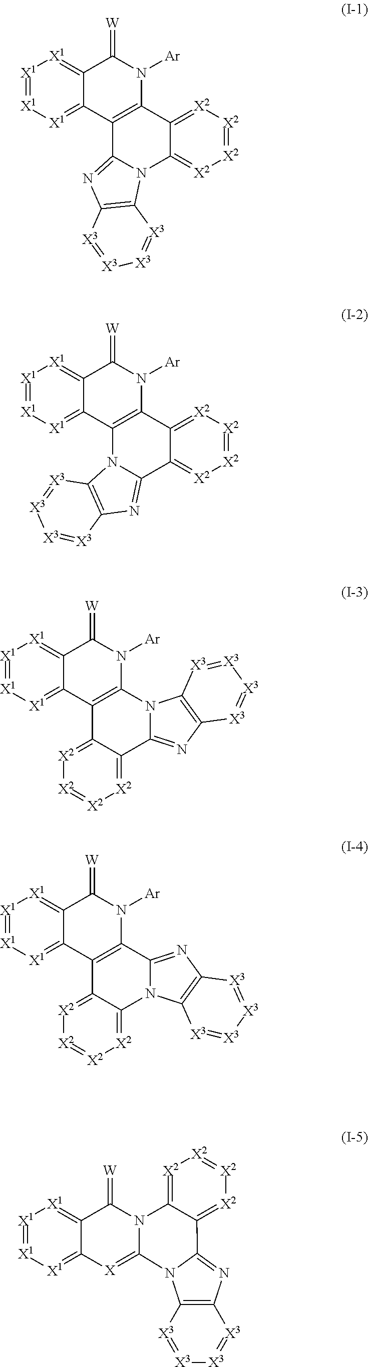

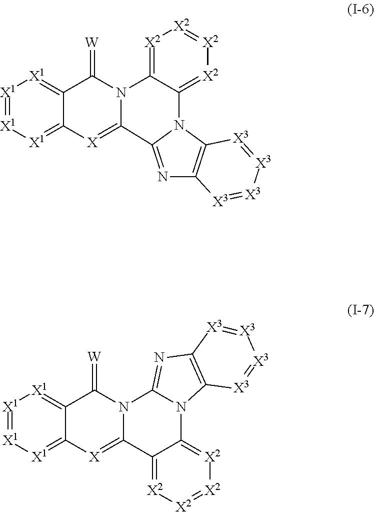

where the symbols W, X, X¹, X², X³ and Ar have the definitions given above, especially for structural elements (A), (B) and (C).

It may further be the case that, in structural element (A) and/or in compounds of the formulae (I-1) to (I-7), not more than one X¹ group is N, preferably all X¹ groups are CR¹, where preferably not more than 3, more preferably not more than 2 and especially preferably not more than 1 of the CR¹ groups that X¹ represents are not the CH group.

In one embodiment of the present invention, it may be the case that, in structural element (B) and/or in compounds of the formulae (I-1) to (I-7), not more than one X² group is N, preferably all X² groups are CR², where preferably not more than 3, more preferably not more than 2 and especially preferably not more than 1 of the CR² groups that X² represents are not the CH group.

It may further be the case that, in structural element (C) and/or in compounds of the formulae (I-1) to (I-7), not more than one X³ group is N, preferably all X³ groups are CR³, where preferably not more than 3, more preferably not more than 2 and especially preferably not more than 1 of the CR³ groups that X³ represents are not the CH group.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5-60 or 5-40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

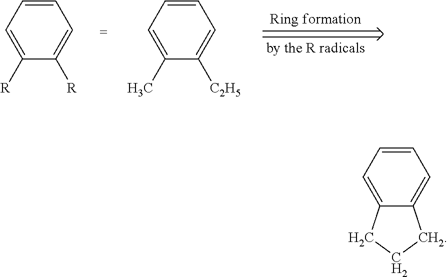

In addition, however, the abovementioned wording shall also be understood to mean that, if one of the two radicals is hydrogen, the second radical binds to the position to which the hydrogen atom was bonded, forming a ring. This will be illustrated by the following scheme:

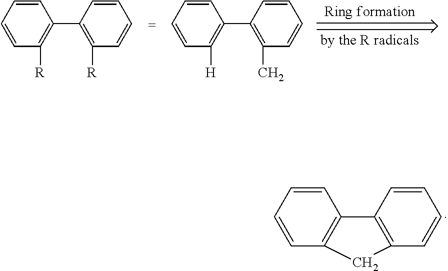

In a preferred configuration, the compounds of the invention may preferably comprise at least one structure of the formulae (II-1) to (II-30) and are more preferably selected from the compounds of the formulae (II-1) to (II-30):

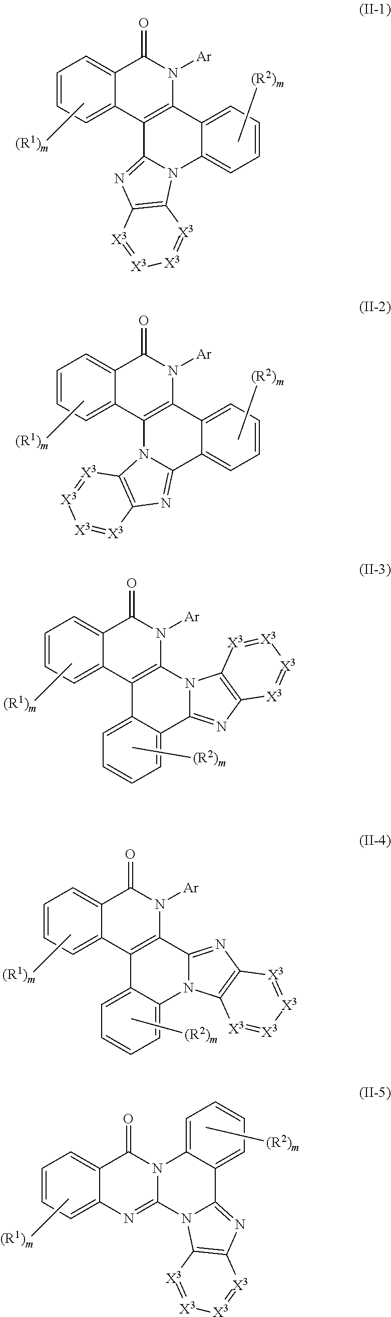

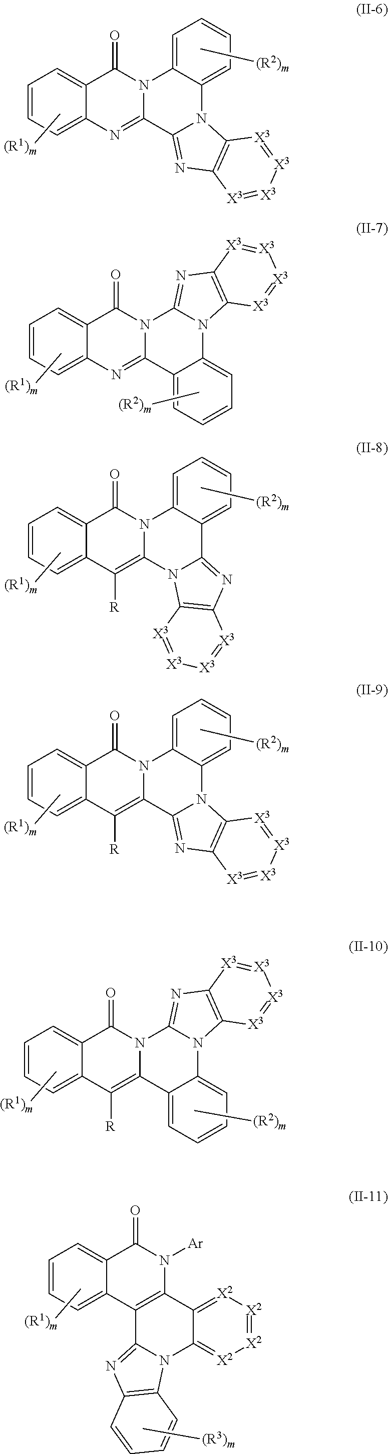

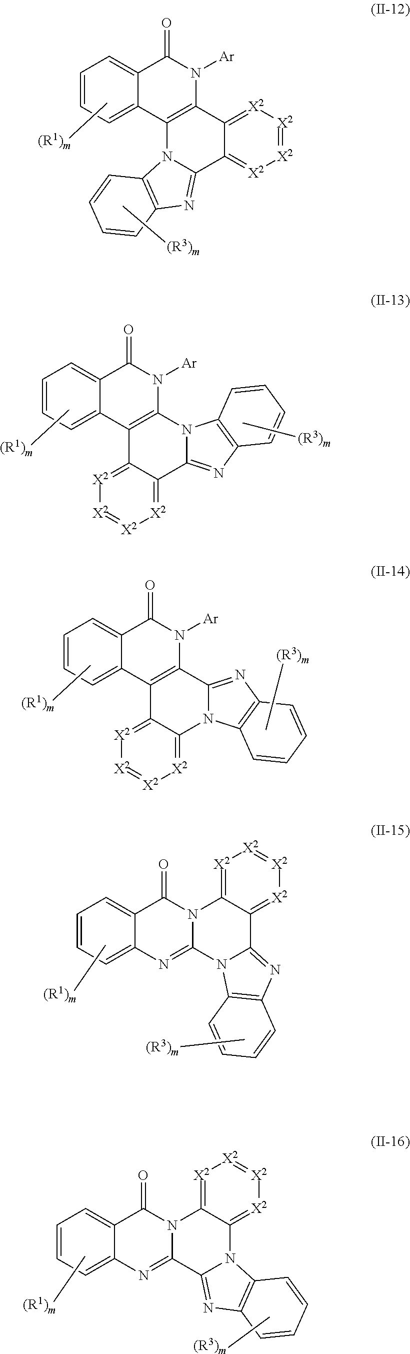

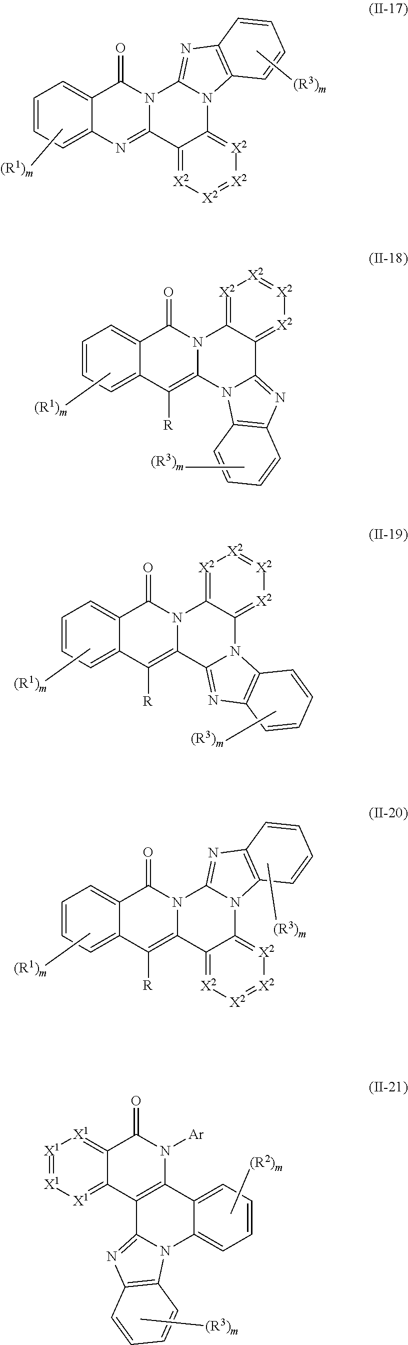

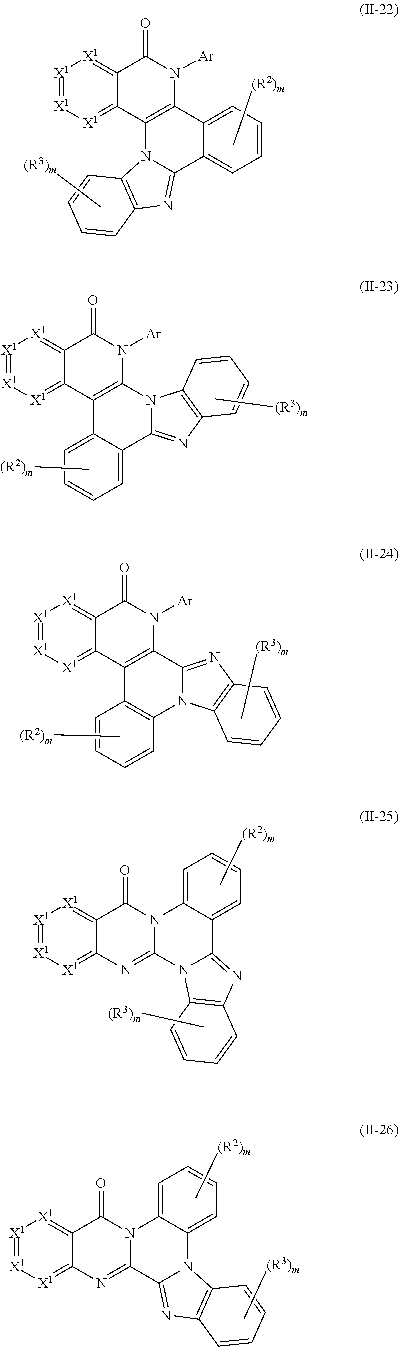

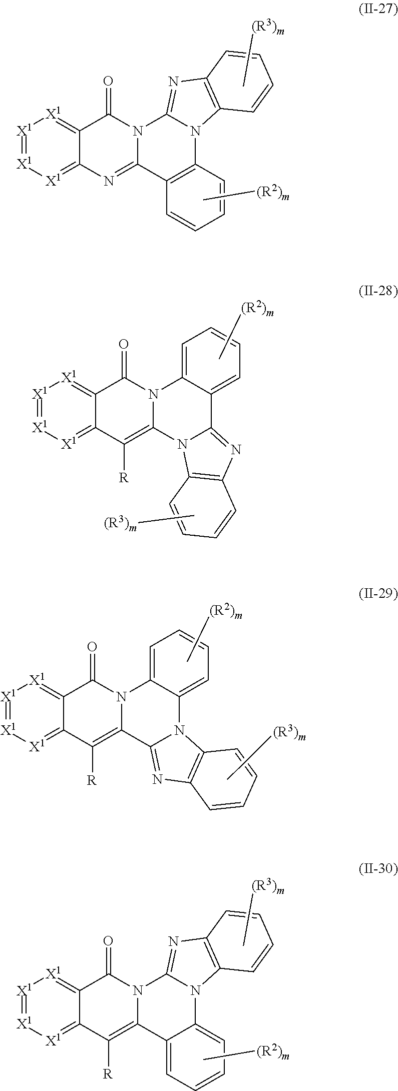

where the symbols X¹, X², X³, R, R¹, R², R³ and Ar have the definitions given above, especially for structural elements (A), (B) and (C), and the index m is 0, 1, 2, 3 or 4, preferably 0, 1 or 2, Preference is given here to structures/compounds of the formulae (II-1) to (II-7), (II-11) to (II-17) and/or (II-21) to (II-27).

In a further preferred embodiment, it may be the case that the compounds of the invention include a structure of the formulae (III-1) to (III-10), where the compounds of the invention may more preferably be selected from the compounds of the formulae (III-1) to (III-10)

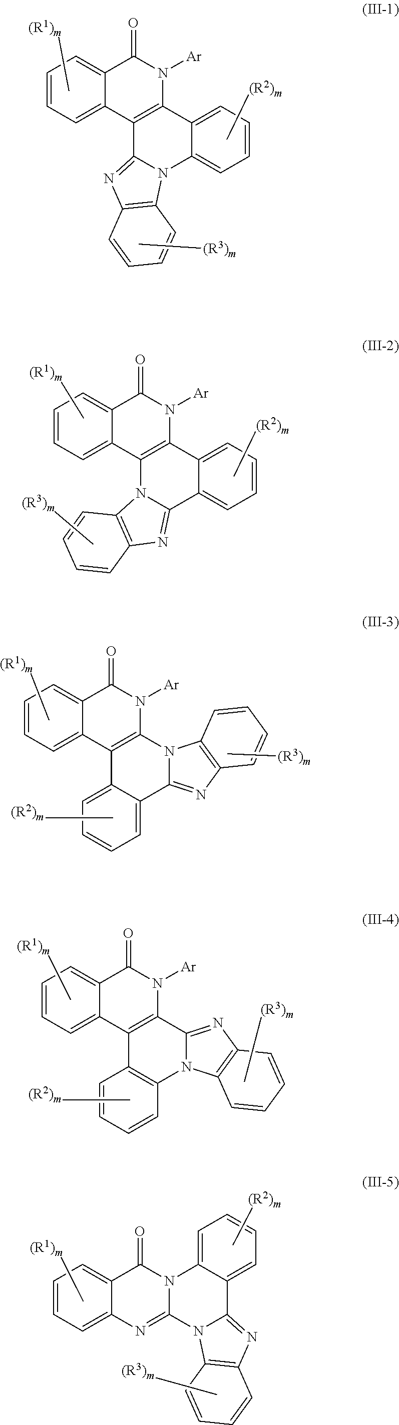

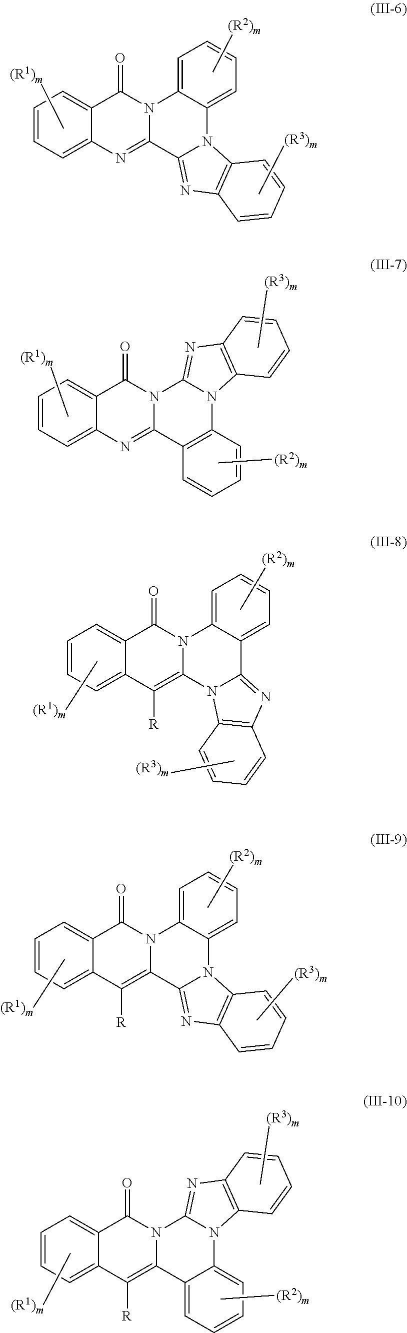

where the symbols R, R¹, R², R³ and Ar have the definitions given above, especially for structural elements (A), (B) and (C), and the index m is 0, 1, 2, 3 or 4, preferably 0, 1 or 2. Preference is given here to structures/compounds of the formulae (III-1) to (III-7).

The index m is 0, 1, 2, 3 or 4, preferably 0, 1 or 2. If the index m is less than 4, the respective rings have a corresponding number of hydrogen atoms. It should be emphasized here that the R¹, R², R³ groups may be H. If, therefore, the index n is not 0, these rings preferably have substituents R¹, R², R³. This means that the corresponding R¹, R², R³ groups are preferably a radical other than H. In this context, the preferences set out above and hereinafter for the corresponding R¹, R², R³ groups are applicable. This clarification is correspondingly applicable to the further groups, radicals, for example R, R⁴, R⁵, R⁶, R⁷, R⁸ and/or R⁹, and indices, especially to n, l and r.

Preferably, the sum total of the indices m is not more than 6, especially preferably not more than 4 and more preferably not more than 2. This is applicable to structures/compounds including those of the formulae (II-1) to (II-30) and (III-1) to (III-10).

It may further be the case that the substituents R, R¹, R² and R³ according to the above formulae do not form a fused aromatic or heteroaromatic ring system, preferably any fused ring system, with the ring atoms of the ring system. This includes the formation of a fused ring system with possible substituents R⁴, R⁵ which may be bonded to the R, R¹, R², R³ radicals.

When two radicals that may especially be selected from R, R², R³, R⁴, R⁵, R⁶, R⁷, R⁸ and/or R⁹ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R¹, R², R³, R⁴, R⁵, R⁶, R⁷, R⁸ and/or R⁹ may also be joined to one another via a bond, such that this can bring about a ring closure.

It may preferably be the case that at least one R, R¹, R² and/or R³ group is the same or different and is selected from the radicals of the following formulae SAr-1 to SAr-18:

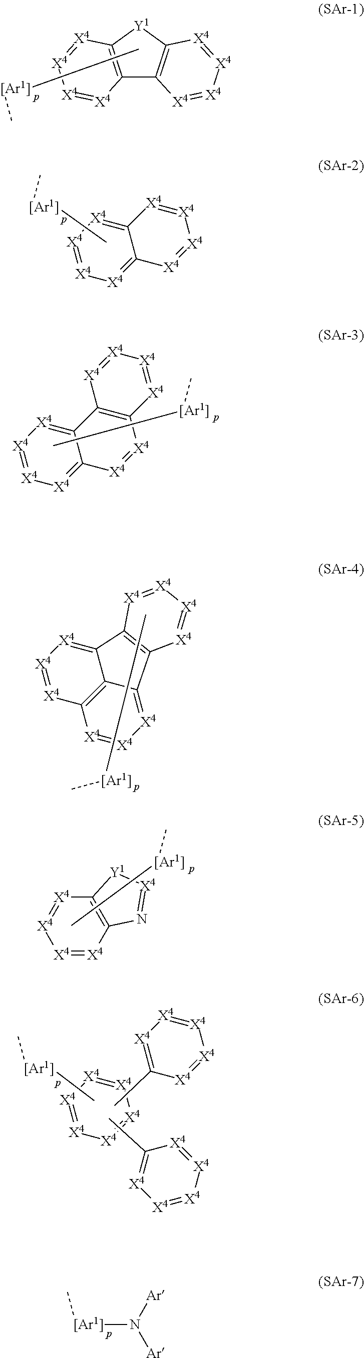

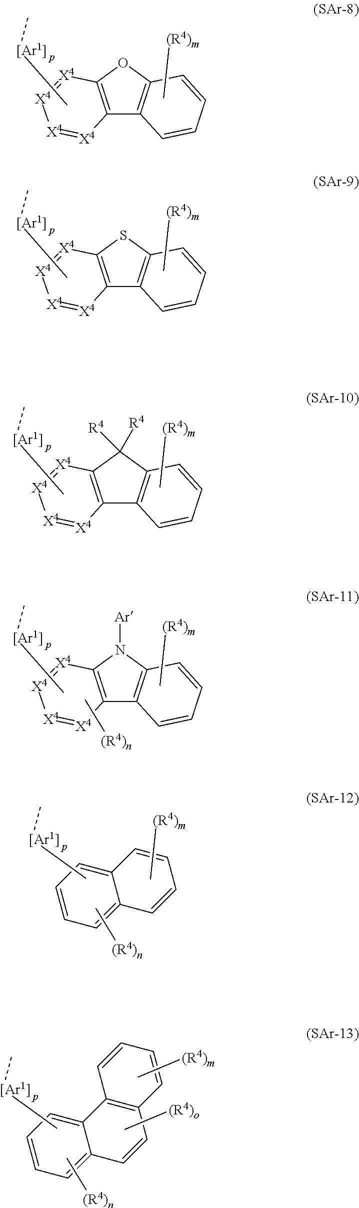

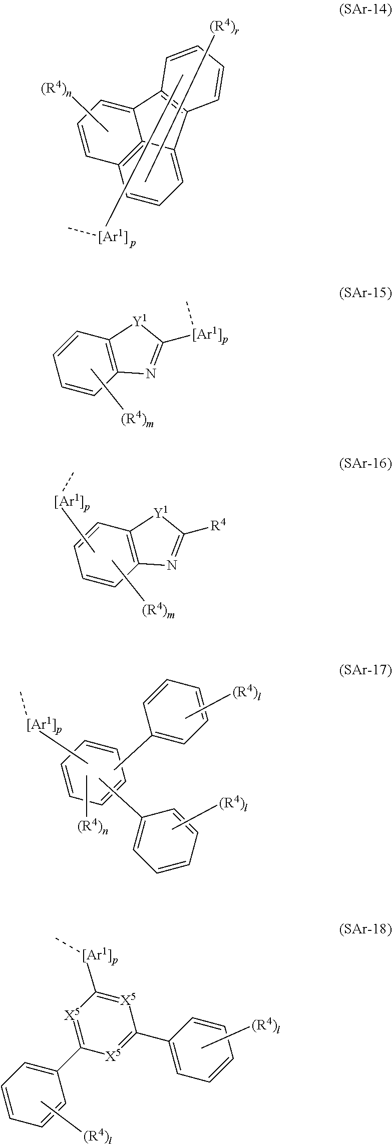

where R⁴ and Ar′ have the definitions given above, especially for structural elements (A), (B) and (C), the dotted bond represents the bond to the corresponding group, and the further symbols and indices are as follows:
X⁴ is the same or different at each instance and is CR⁴, N, or C if the [Ar¹]ₚ group binds thereto, preferably CR⁴, where there are preferably no N—N bonds;
X⁵ is the same or different at each instance and is CR⁴ or N, preferably N;
Ar¹ is the same or different at each instance and is a bivalent aromatic or heteroaromatic ring system which has 6 to 18 aromatic ring atoms and in each case has R⁴ radicals;
Y¹ is the same or different at each instance and is C(R⁴)₂, NR⁴, O, S, or N if the [Ar¹]ₚ group binds thereto;
p is 0 or 1, where p=0 means that the Ar¹ group is absent and that the corresponding aromatic or heteroaromatic group is bonded directly to the corresponding radical;
n is 0, 1, 2 or 3, preferably 0, 1 or 2;
m is 0, 1, 2, 3 or 4, preferably 0, 1 or 2;
l is 0, 1, 2, 3, 4 or 5, preferably 0, 1 or 2;
r is 0, 1, 2, 3, 4, 5 or 6, preferably 0, 1 or 2.

Preference is given here to structures of the formulae (SAr-1), (SAr-4), (SAr-8), (SAr-10), (SAr-11), (SAr-14), (SAr-18).

It may further be the case that R, R¹, R² and/or R³ are the same or different at each instance and are selected from the group consisting of H, D or an aromatic or heteroaromatic ring system selected from the groups of the following formulae Ar-1 to Ar-79; preferably, at least one R, R¹, R² and/or R³ group is the same or different and is selected from the groups of the following formulae Ar-1 to Ar-79 and/or the Ar′ group is the same or different at each instance and is selected from the groups of the following formulae Ar-1 to Ar-79:

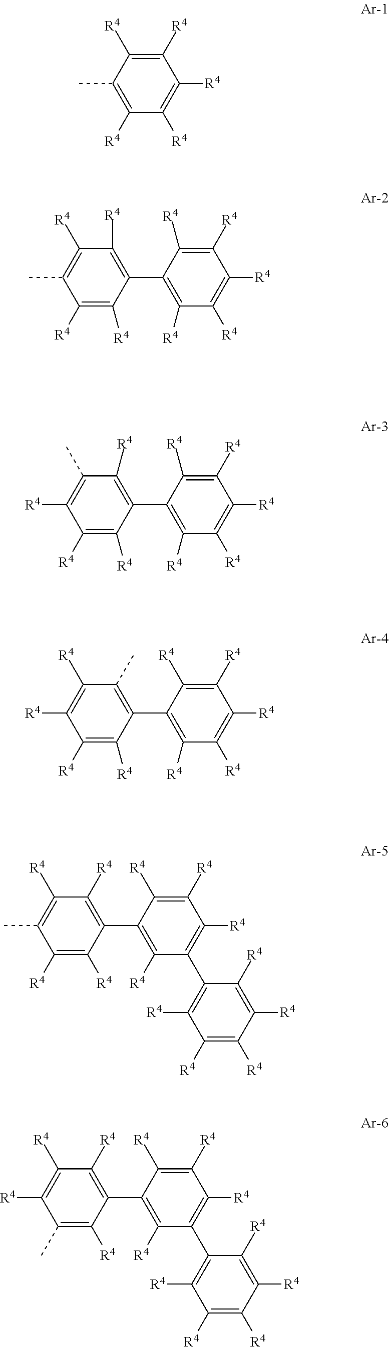

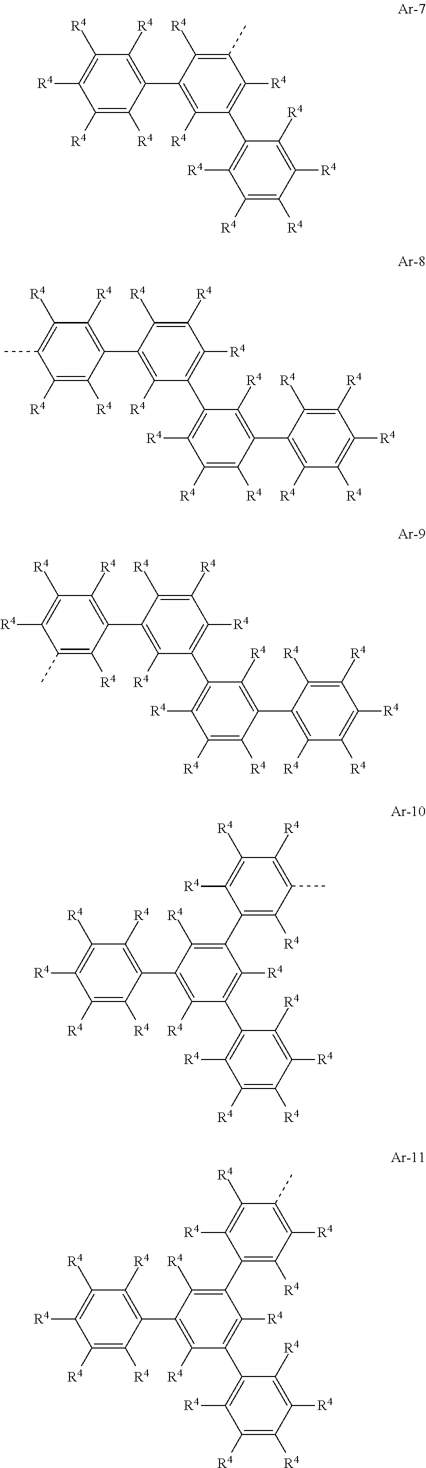

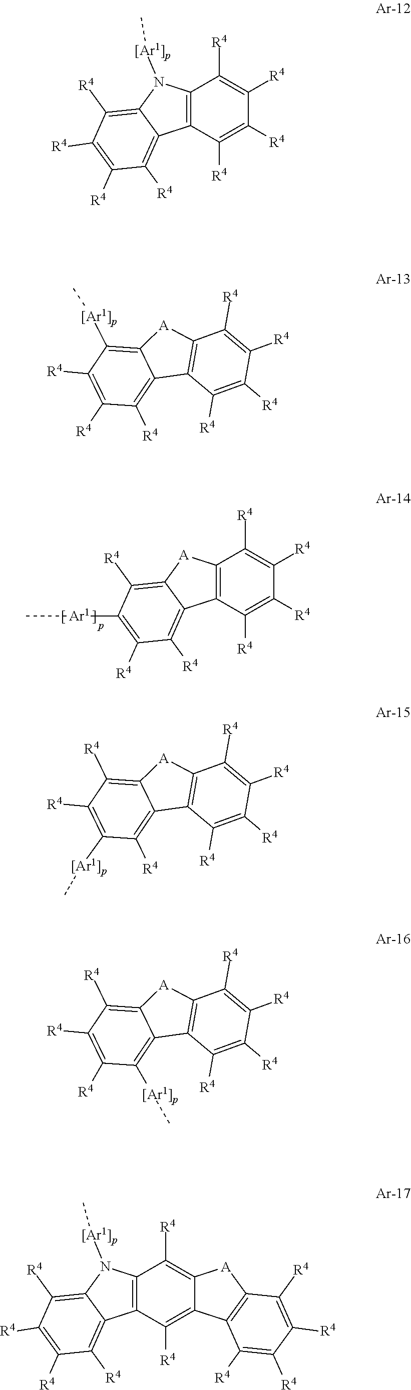

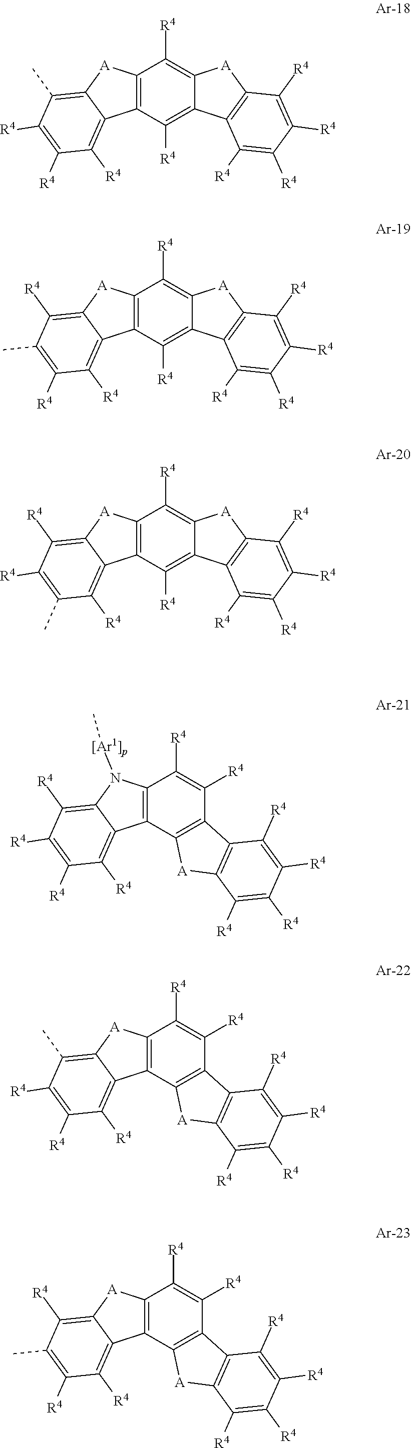

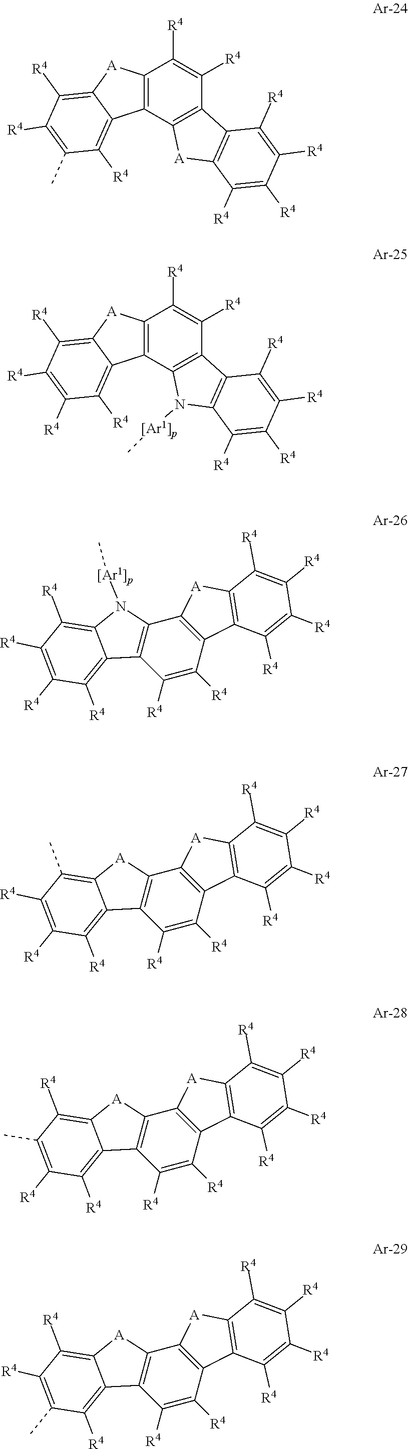

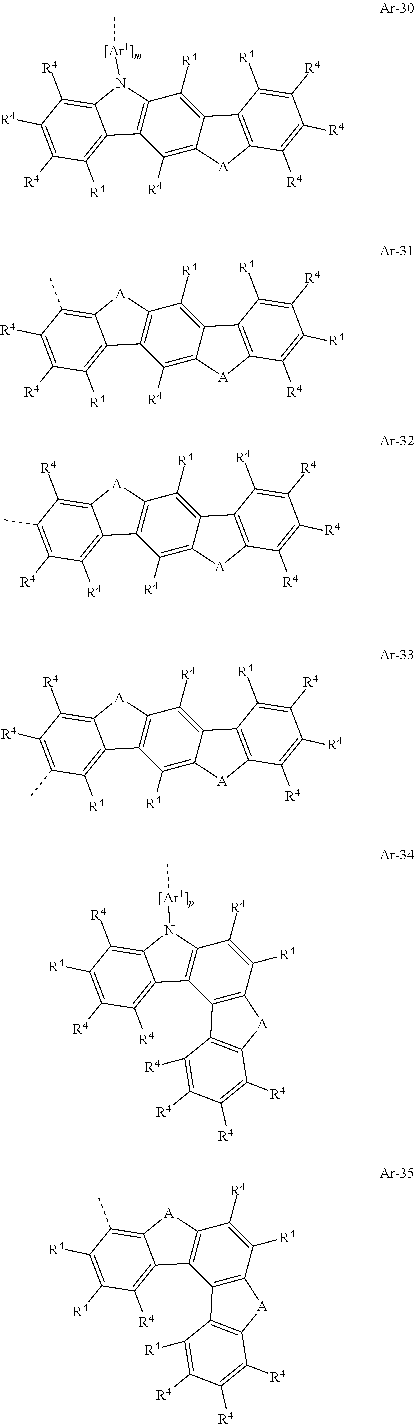

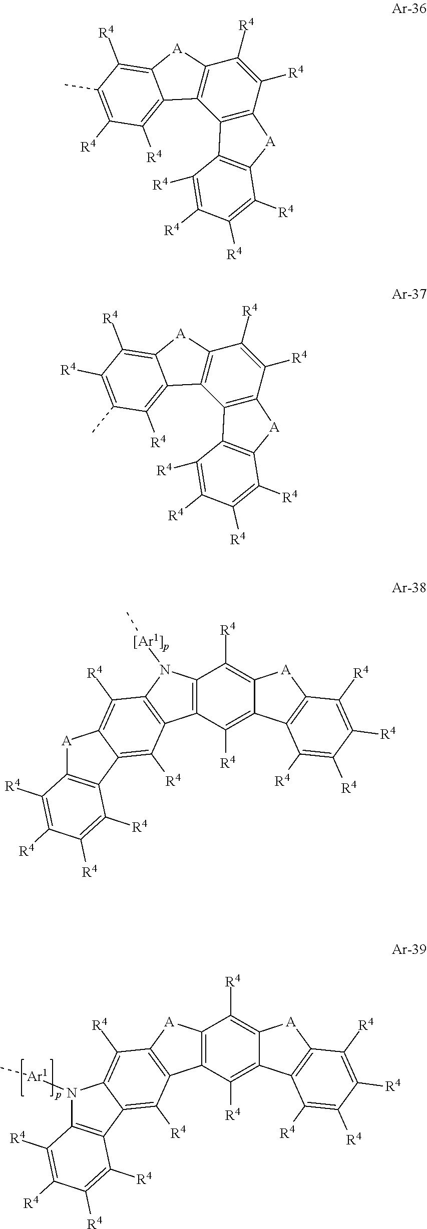

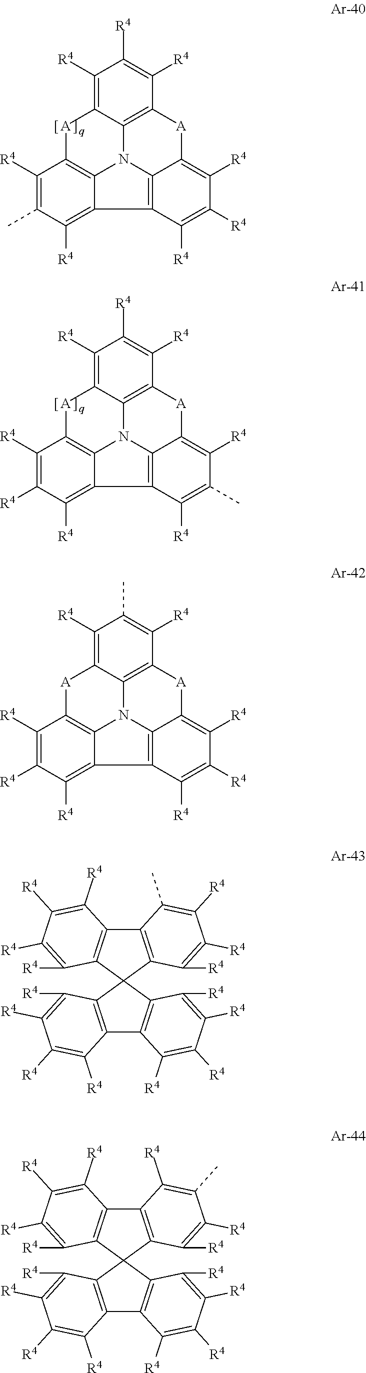

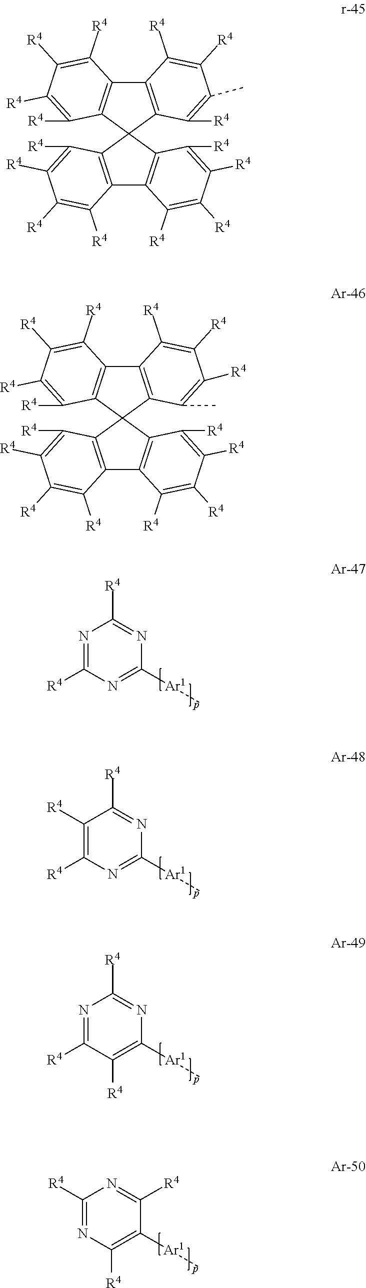

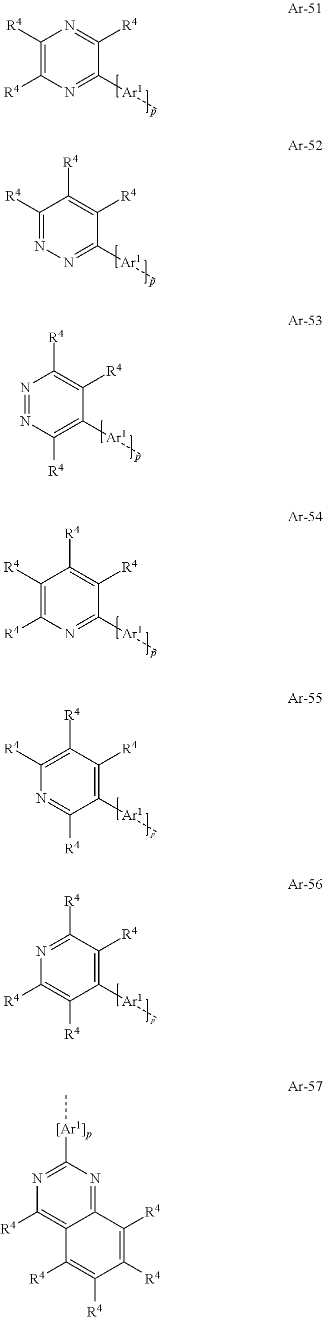

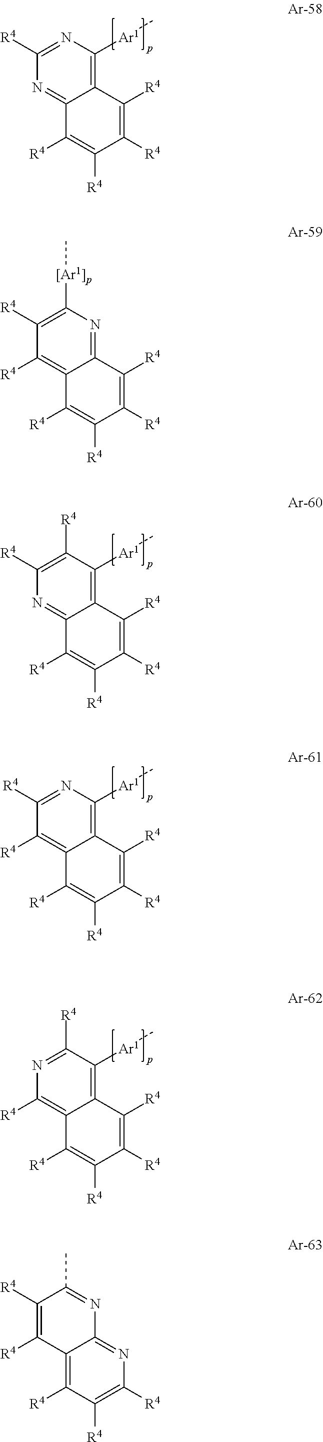

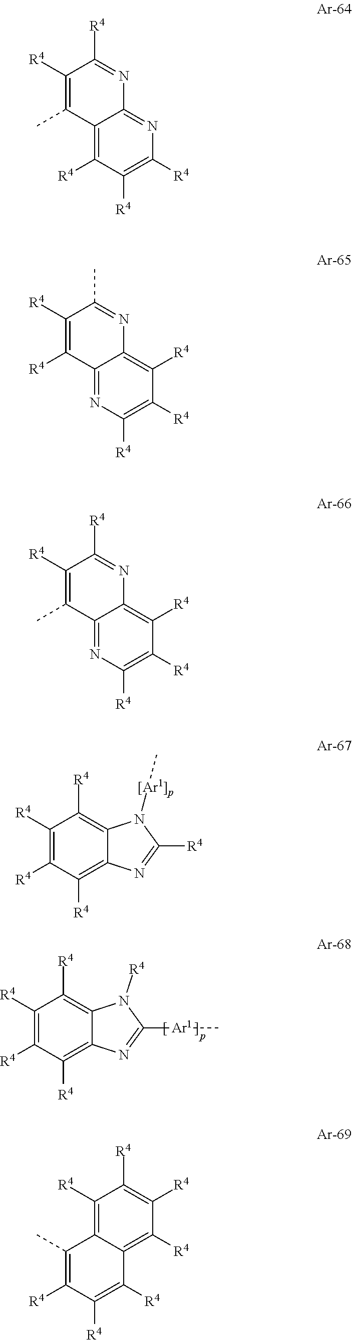

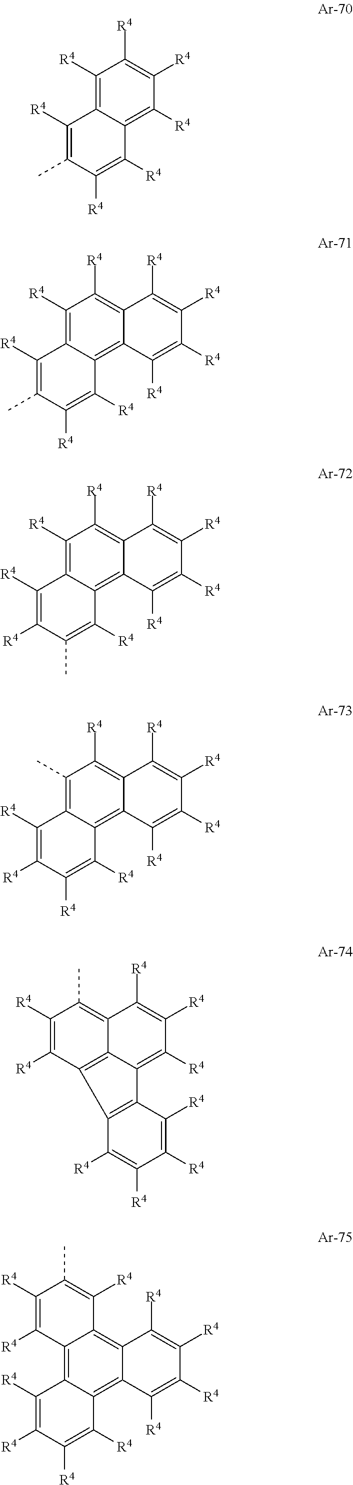

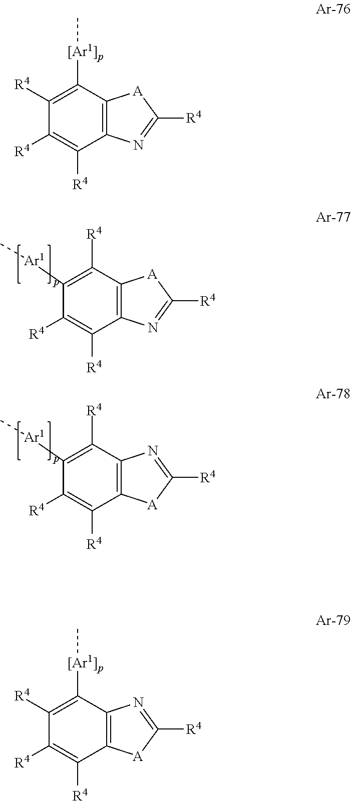

where R⁴ has the definitions given above, the dotted bond represents the bond to the corresponding group and in addition:
Ar¹ is the same or different at each instance and is a bivalent aromatic or heteroaromatic ring system which has 6 to 18 aromatic ring atoms and in each case has R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

Preference is given here to structures of the formulae (Ar-1), (Ar-2), (Ar-3), (Ar-12), (Ar-13), (Ar-14), (Ar-15), (Ar-16), (Ar-47), (Ar-70), (Ar-75), (Ar-76), (Ar-77), (Ar-78), (Ar-79), particular preference to structures of the formulae (Ar-1), (Ar-2), (Ar-3), (Ar-12), (Ar-13), (Ar-14), (Ar-15), (Ar-16).

When the abovementioned groups for R, R¹, R², R³ have two or more A groups, possible options for these include all combinations from the definition of A. Preferred embodiments in that case are those in which one A group is NR⁴ and the other A group is C(R⁴)₂ or in which both A groups are NR⁴ or in which both A groups are O.

When A is NR⁴, the substituent R⁴ bonded to the nitrogen atom is preferably an aromatic or heteroaromatic ring system which has 5 to 24 aromatic ring atoms and may also be substituted by one or more R⁵ radicals. In a particularly preferred embodiment, this R⁴ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁵ radicals. Preference is given to phenyl, biphenyl, terphenyl and quaterphenyl having bonding patterns as listed above for Ar-1 to Ar-11, where these structures, rather than by R⁴, may be substituted by one or more R⁵ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁵ radicals.

When A is C(R⁴)₂, the substituents R⁴ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁴ is a methyl group or a phenyl group. In this case, the R⁴ radicals together may also form a ring system, which leads to a spiro system.

It may preferably be the case that Ar is the same or different at each instance and is selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R radicals. In particular, the Ar group is present in structural element (A) if Z¹ is NAr. In addition, this group is detailed explicitly in formulae (I-1) to (I-4) inter alia and preferred embodiments of these formulae. Particularly preferred Ar groups are the structures Ar-1 to Ar-79 shown above, preference being given to structures of the formulae (Ar-1), (Ar-2), (Ar-3), (Ar-12), (Ar-13), (Ar-14), (Ar-15), (Ar-16), (Ar-47), (Ar-70), (Ar-75), (Ar-76), (Ar-77), (Ar-78), (Ar-79), and particular preference to structures of the formulae (Ar-1), (Ar-2), (Ar-3), (Ar-12), (Ar-13), (Ar-14), (Ar-15), (Ar-16). In the structures Ar-1 to Ar-79 set out above, in relation to the Ar radicals, the substituents R⁴ should be replaced by the corresponding R radicals.

There follows a description of preferred substituents R, R¹, R² and R³.

In a preferred embodiment of the invention, R, R¹, R² and R³ are the same or different at each instance and are selected from the group consisting of H, D, F, CN, NO₂, Si(R⁴)₃, B(OR⁴)₂, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and are selected from the group consisting of H, D, F, a straight-chain alkyl group having 1 to 20 carbon atoms or a branched or cyclic alkyl group having 3 to 20 carbon atoms, where the alkyl group may be substituted in each case by one or more R4 radicals, or an aromatic or heteroaromatic ring system which has 5 to 60 aromatic ring atoms, preferably 5 to 40 aromatic ring atoms, and may be substituted in each case by one or more R4 radicals.

In a further-preferred embodiment of the invention, R, R¹, R² and R³ are the same or different at each instance and is selected from the group consisting of H, D, an aromatic or heteroaromatic ring system which has 6 to 30 aromatic ring atoms and may be substituted by one or more R4 radicals, and an N(Ar)₂ group. More preferably, R, R¹, R² are the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁴ radicals.

Preferred aromatic or heteroaromatic ring systems that the R, R¹, R², R³ and Ar′ may preferably represent are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁴ radicals. Particular preference is given to the structures Ar-1 to Ar-79 shown above, preference being given to structures of the formulae (Ar-1), (Ar-2), (Ar-3), (Ar-12), (Ar-13), (Ar-14), (Ar-15), (Ar-16), (Ar-47), (Ar-70), (Ar-75), (Ar-76), (Ar-77), (Ar-78), (Ar-79), and particular preference to structures of the formulae (Ar-1), (Ar-2), (Ar-3), (Ar-12), (Ar-13), (Ar-14), (Ar-15), (Ar-16).

It may further be the case that two adjacent X¹, X², X³ groups are CR¹, CR² or CR³, preferably two adjacent X², X³ groups are CR² or CR³, where the two adjacent substituents R¹, R², R³ form a fused aromatic or heteroaromatic ring system which has 5 to 40 aromatic ring atoms and may be substituted by one or more R⁴ radicals, forming, together with the ring atoms of the 6-membered rings substituted by the R¹, R², R³ groups, a fused ring system having at least two rings which is preferably selected from naphthalene, indole, carbazole, dibenzofuran, dibenzothiophene, indenocarbazole, indolocarbazole, quinoline, isoquinoline, quinazoline, quinoxaline, phenanthrene and triphenylene.

Further suitable R, R¹, R² and R³ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40.

Ar⁴ and Ar² here may also be bonded to one another and/or Ar² and Ar³ to one another by a group selected from C(R⁴)₂, NR⁴, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R⁴ radicals. More preferably, Ar⁴ is selected from the group consisting of ortho-, meta- or para-phenylene or ortho-, meta- or para-biphenyl, each of which may be substituted by one or more R⁴ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R⁴ radicals.

Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R⁴ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R⁵ radicals. In a particularly preferred embodiment of the invention, R⁴ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁵ radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R⁵ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

It may further be the case that the compound comprises exactly two or exactly three structures of formulae (I-1) to (I-7), (II-1) to (II-30) and/or (III-1) to (III-10), where preferably one of the aromatic or heteroaromatic ring systems that can be represented by at least one of the R, R¹, R², R³ groups or to which the R, R¹, R², R³ groups bind is shared by the two structures. It may additionally be the case that the compound comprises a connecting group via which the exactly two or three structures of formulae (1-1) to (I-7), (II-1) to (II-30) and/or (III-1) to (III-10) are bonded to one another. These connecting groups are preferably derived from groups that are defined for the R, R¹, R², R³ groups, but where one or two hydrogen atoms should be replaced by bonding sites. In a further configuration, an inventive compound comprising structures of formulae (I-1) to (I-7), (II-1) to (II-30) and/or (III-1) to (III-10) may be configured as an oligomer, polymer or dendrimer, where, in place of one hydrogen atom or one substituent, there are one or more bonds of the compounds to the polymer, oligomer or dendrimer.

When the compounds structural elements (A), (B) and (C) and/or compound comprising structures of formulae (I-1) to (I-7) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene, which, because of their high triplet energy, may be preferable in spite of the presence of fused aromatic six-membered rings.

In addition, it is a feature of preferred compounds of the invention that they are sublimable. These compounds generally have a molar mass of less than about 1200 g/mol.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

| | |
|---|---|
| 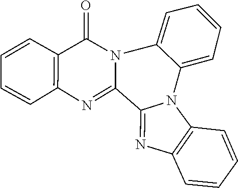 [SMILES: O=c1c2ccccc2nc2c3nc4ccccc4n3c3ccccc3n12] | 1 |
| 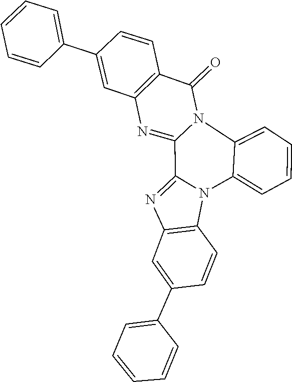 [SMILES: O=c1c2ccc(-c3ccccc3)cc2nc2c3nc4cc(-c5ccccc5)ccc4n3c3ccccc3n12] | 2 |
| 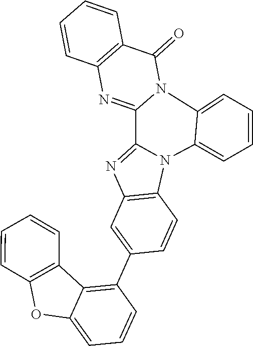 [SMILES: O=c1c2ccccc2nc2c3nc4cc(-c5cccc6oc7ccccc7c56)ccc4n3c3ccccc3n12] | 3 |
| 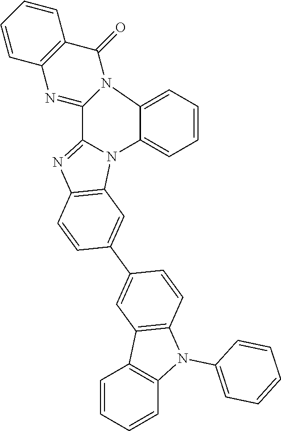 [SMILES: O=c1c2ccccc2nc2c3nc4ccc(-c5ccc6c(c5)c5ccccc5n6-c5ccccc5)cc4n3c3ccccc3n12] | 4 |
| 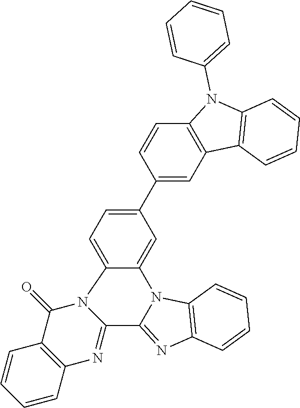 [SMILES: O=c1c2ccccc2nc2c3nc4ccccc4n3c3cc(-c4ccc5c(c4)c4ccccc4n5-c4ccccc4)ccc3n12] | 5 |
| 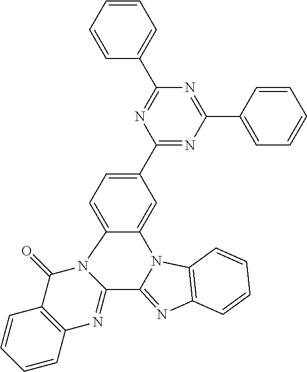 [SMILES: O=c1c2ccccc2nc2c3nc4ccccc4n3c3cc(-c4nc(-c5ccccc5)nc(-c5ccccc5)n4)ccc3n12] | 6 |
| 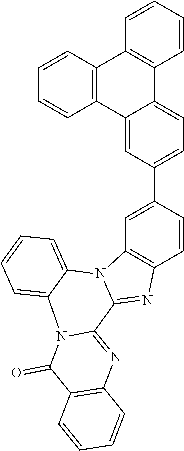 [SMILES: O=c1c2ccccc2nc2c3nc4ccc(-c5ccc6c7ccccc7c7ccccc7c6c5)cc4n3c3ccccc3n12] | 7 |
| 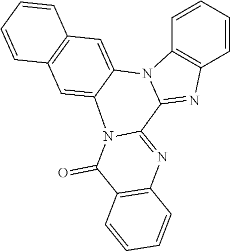 [SMILES: O=c1c2ccccc2nc2c3nc4ccccc4n3c3cc4ccccc4cc3n12] | 8 |
| 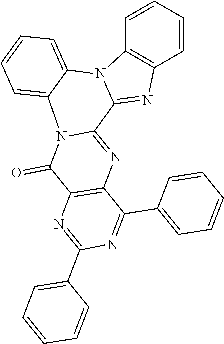 [SMILES: O=c1c2nc(-c3ccccc3)nc(-c3ccccc3)c2nc2c3nc4ccccc4n3c3ccccc3n12] | 9 |
| 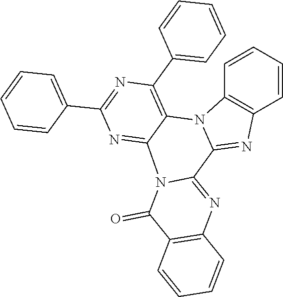 [SMILES: O=c1c2ccccc2nc2c3nc4ccccc4n3c3c(-c4ccccc4)nc(-c4ccccc4)nc3n12] | 10 |
| 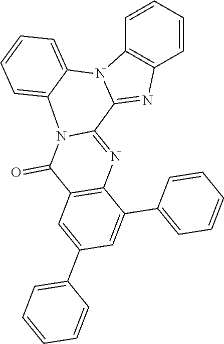 [SMILES: O=c1c2cc(-c3ccccc3)cc(-c3ccccc3)c2nc2c3nc4ccccc4n3c3ccccc3n12] | 11 |
| 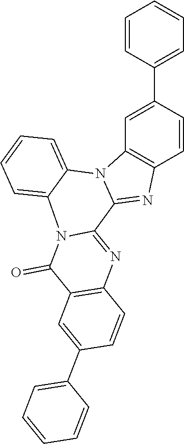 [SMILES: O=c1c2cc(-c3ccccc3)ccc2nc2c3nc4ccc(-c5ccccc5)cc4n3c3ccccc3n12] | 12 |
| 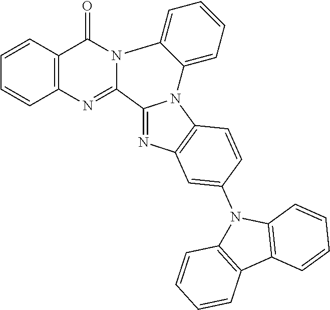 [SMILES: O=c1c2ccccc2nc2c3nc4cc(-n5c6ccccc6c6ccccc65)ccc4n3c3ccccc3n12] | 13 |
| 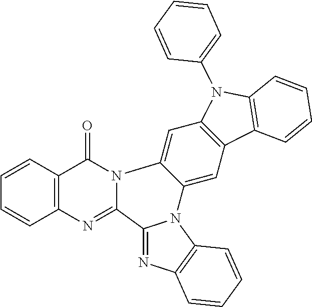 [SMILES: O=c1c2ccccc2nc2c3nc4ccccc4n3c3cc4c5ccccc5n(-c5ccccc5)c4cc3n12] | 14 |
| 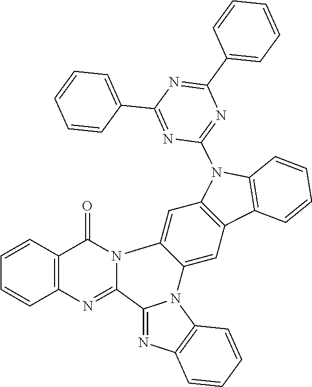 [SMILES: O=c1c2ccccc2nc2c3nc4ccccc4n3c3cc4c5ccccc5n(-c5nc(-c6ccccc6)nc(-c6ccccc6)n5)c4cc3n12] | 15 |
| 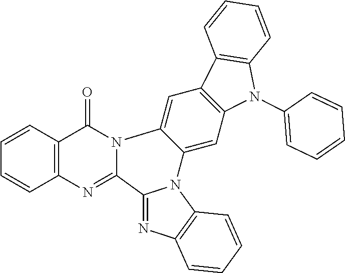 [SMILES: O=c1c2ccccc2nc2c3nc4ccccc4n3c3cc4c(cc3n12)c1ccccc1n4-c1ccccc1] | 16 |
| 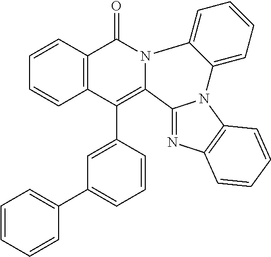 [SMILES: O=c1c2ccccc2c(-c2cccc(-c3ccccc3)c2)c2c3nc4ccccc4n3c3ccccc3n12] | 17 |
| 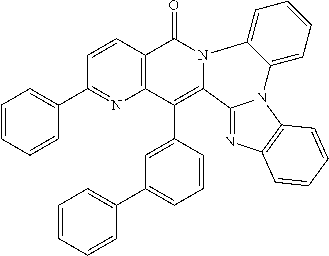 [SMILES: O=c1c2ccc(-c3ccccc3)nc2c(-c2cccc(-c3ccccc3)c2)c2c3nc4ccccc4n3c3ccccc3n12] | 18 |
| 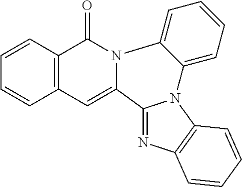 [SMILES: O=c1c2ccccc2cc2c3nc4ccccc4n3c3ccccc3n12] | 19 |
| 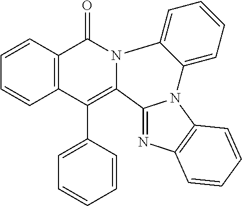 [SMILES: O=c1c2ccccc2c(-c2ccccc2)c2c3nc4ccccc4n3c3ccccc3n12] | 20 |
| 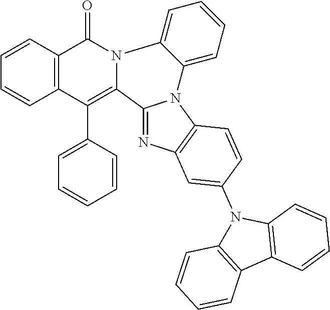 [SMILES: O=c1c2ccccc2c(-c2ccccc2)c2c3nc4cc(-n5c6ccccc6c6ccccc65)ccc4n3c3ccccc3n12] | 21 |
| 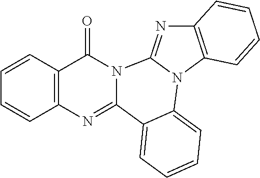 [SMILES: O=c1c2ccccc2nc2c3ccccc3n3c4ccccc4nc3n12] | 22 |
| 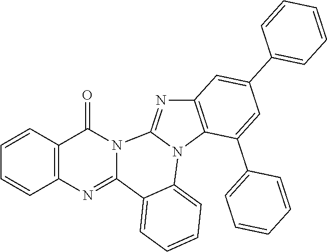 [SMILES: O=c1c2ccccc2nc2c3ccccc3n3c4c(-c5ccccc5)cc(-c5ccccc5)cc4nc3n12] | 23 |
| 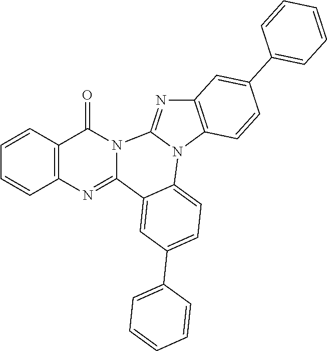 [SMILES: O=c1c2ccccc2nc2c3cc(-c4ccccc4)ccc3n3c4ccc(-c5ccccc5)cc4nc3n12] | 24 |
| 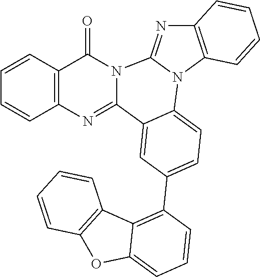 [SMILES: O=c1c2ccccc2nc2c3cc(-c4cccc5oc6ccccc6c45)ccc3n3c4ccccc4nc3n12] | 25 |
| 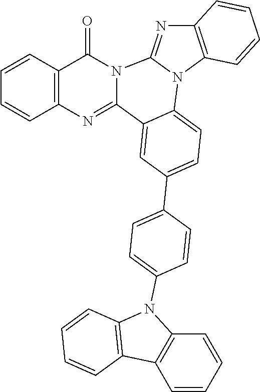 [SMILES: O=c1c2ccccc2nc2c3cc(-c4ccc(-n5c6ccccc6c6ccccc65)cc4)ccc3n3c4ccccc4nc3n12] | 26 |
| 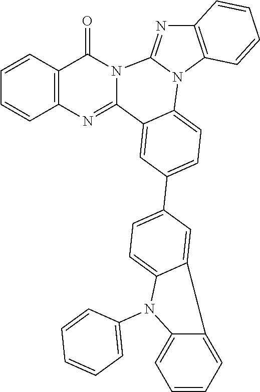 [SMILES: O=c1c2ccccc2nc2c3cc(-c4ccc5c(c4)c4ccccc4n5-c4ccccc4)ccc3n3c4ccccc4nc3n12] | 27 |
| 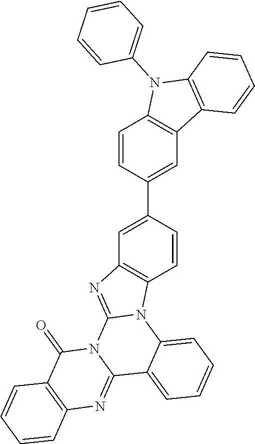 [SMILES: O=c1c2ccccc2nc2c3ccccc3n3c4ccc(-c5ccc6c(c5)c5ccccc5n6-c5ccccc5)cc4nc3n12] | 28 |
| 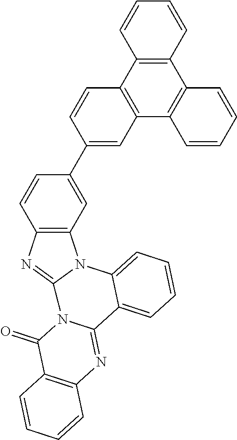 [SMILES: O=c1c2ccccc2nc2c3ccccc3n3c4cc(-c5ccc6c7ccccc7c7ccccc7c6c5)ccc4nc3n12] | 29 |
| 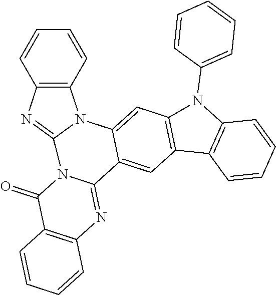 [SMILES: O=c1c2ccccc2nc2c3cc4c5ccccc5n(-c5ccccc5)c4cc3n3c4ccccc4nc3n12] | 30 |
| 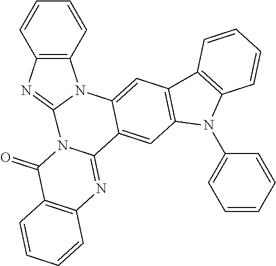 [SMILES: O=c1c2ccccc2nc2c3cc4c(cc3n3c5ccccc5nc3n12)c1ccccc1n4-c1ccccc1] | 31 |
| 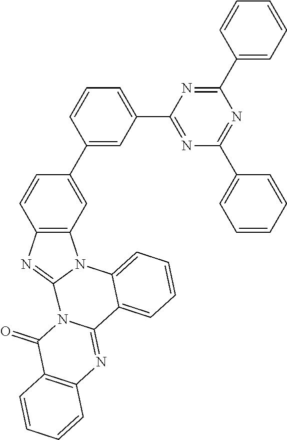 [SMILES: O=c1c2ccccc2nc2c3ccccc3n3c4cc(-c5cccc(-c6nc(-c7ccccc7)nc(-c7ccccc7)n6)c5)ccc4nc3n12] | 32 |
| 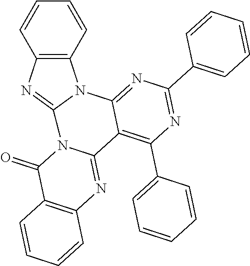 [SMILES: O=c1c2ccccc2nc2c3c(-c4ccccc4)nc(-c4ccccc4)nc3n3c4ccccc4nc3n12] | 33 |
| 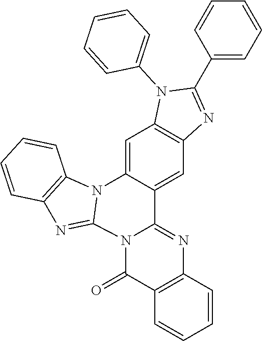 [SMILES: O=c1c2ccccc2nc2c3cc4nc(-c5ccccc5)n(-c5ccccc5)c4cc3n3c4ccccc4nc3n12] | 34 |
| 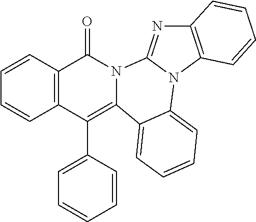 [SMILES: O=c1c2ccccc2c(-c2ccccc2)c2c3ccccc3n3c4ccccc4nc3n12] | 35 |
| 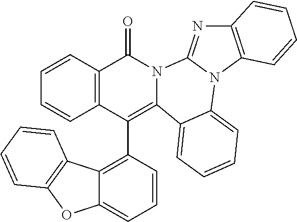 [SMILES: O=c1c2ccccc2c(-c2cccc3oc4ccccc4c23)c2c3ccccc3n3c4ccccc4nc3n12] | 36 |
| 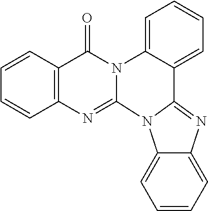 [SMILES: O=c1c2ccccc2nc2n1c1ccccc1c1nc3ccccc3n12] | 37 |
| 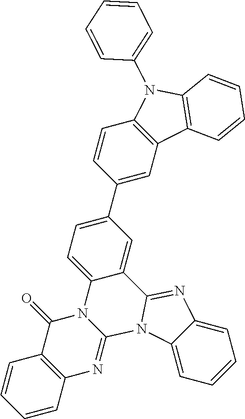 [SMILES: O=c1c2ccccc2nc2n1c1ccc(-c3ccc4c(c3)c3ccccc3n4-c3ccccc3)cc1c1nc3ccccc3n12] | 38 |
| 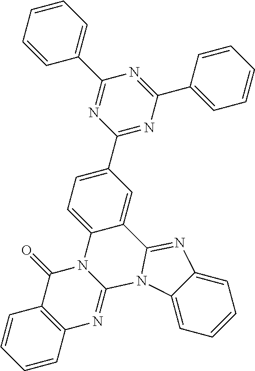 [SMILES: O=c1c2ccccc2nc2n1c1ccc(-c3nc(-c4ccccc4)nc(-c4ccccc4)n3)cc1c1nc3ccccc3n12] | 39 |
| 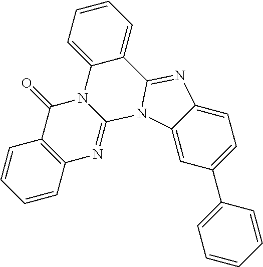 [SMILES: O=c1c2ccccc2nc2n1c1ccccc1c1nc3ccc(-c4ccccc4)cc3n12] | 40 |
| 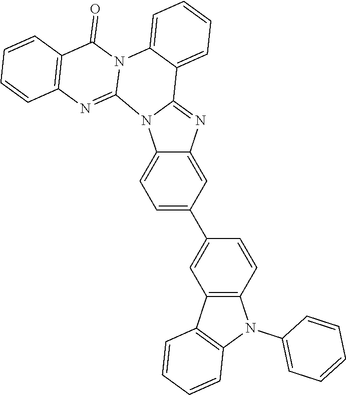 [SMILES: O=c1c2ccccc2nc2n1c1ccccc1c1nc3cc(-c4ccc5c(c4)c4ccccc4n5-c4ccccc4)ccc3n12] | 41 |
| 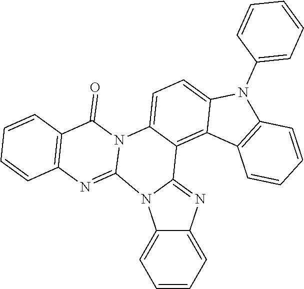 [SMILES: O=c1c2ccccc2nc2n1c1ccc3c(c4ccccc4n3-c3ccccc3)c1c1nc3ccccc3n12] | 42 |
| 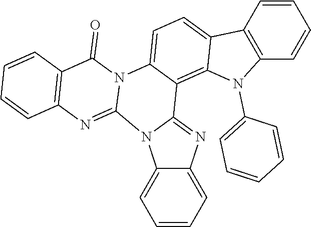 [SMILES: O=c1c2ccccc2nc2n1c1ccc3c4ccccc4n(-c4ccccc4)c3c1c1nc3ccccc3n12] | 43 |
| 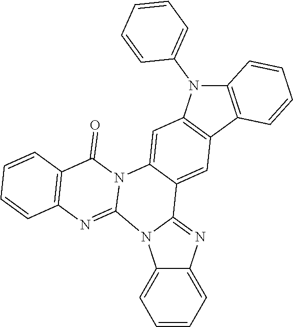 [SMILES: O=c1c2ccccc2nc2n1c1cc3c(cc1c1nc4ccccc4n12)c1ccccc1n3-c1ccccc1] | 44 |
| 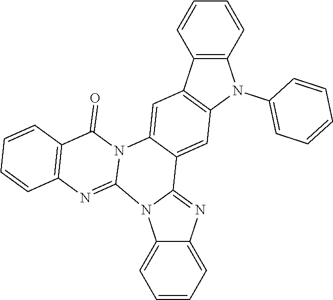 [SMILES: O=c1c2ccccc2nc2n1c1cc3c4ccccc4n(-c4ccccc4)c3cc1c1nc3ccccc3n12] | 45 |
| 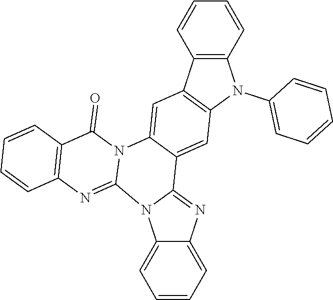 [SMILES: O=c1c2ccccc2nc2n1c1cc3c4ccccc4n(-c4ccccc4)c3cc1c1nc3ccccc3n12] | 46 |
| 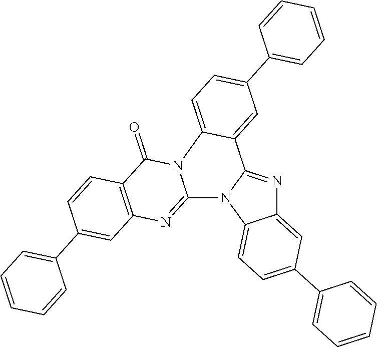 [SMILES: O=c1c2ccc(-c3ccccc3)cc2nc2n1c1ccc(-c3ccccc3)cc1c1nc3cc(-c4ccccc4)ccc3n12] | 47 |
| 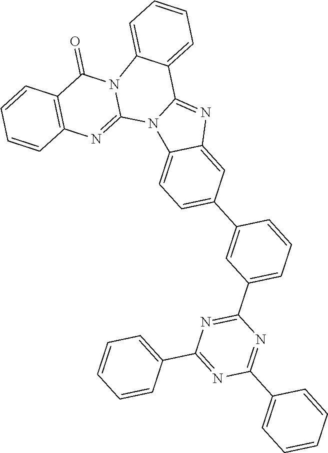 [SMILES: O=c1c2ccccc2nc2n1c1ccccc1c1nc3cc(-c4cccc(-c5nc(-c6ccccc6)nc(-c6ccccc6)n5)c4)ccc3n12] | 48 |
| 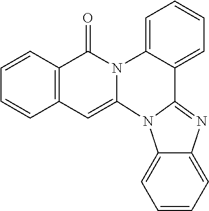 [SMILES: O=c1c2ccccc2cc2n1c1ccccc1c1nc3ccccc3n12] | 49 |
| 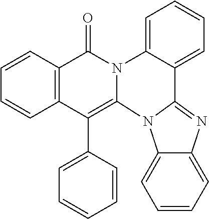 [SMILES: O=c1c2ccccc2c(-c2ccccc2)c2n1c1ccccc1c1nc3ccccc3n12] | 50 |
| 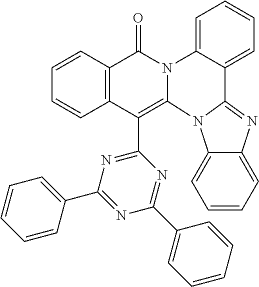 [SMILES: O=c1c2ccccc2c(-c2nc(-c3ccccc3)nc(-c3ccccc3)n2)c2n1c1ccccc1c1nc3ccccc3n12] | 51 |
| 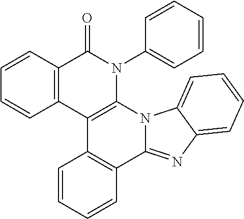 [SMILES: O=c1c2ccccc2c2c3ccccc3c3nc4ccccc4n3c2n1-c1ccccc1] | 52 |
| 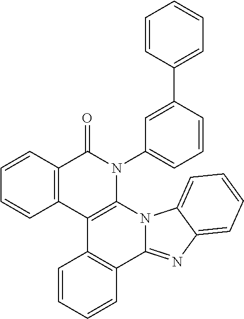 [SMILES: O=c1c2ccccc2c2c3ccccc3c3nc4ccccc4n3c2n1-c1cccc(-c2ccccc2)c1] | 53 |
| 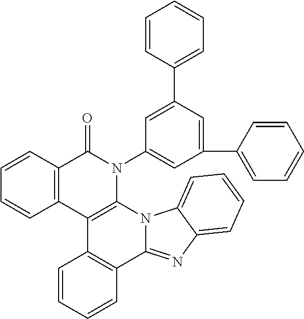 [SMILES: O=c1c2ccccc2c2c3ccccc3c3nc4ccccc4n3c2n1-c1cc(-c2ccccc2)cc(-c2ccccc2)c1] | 54 |
| 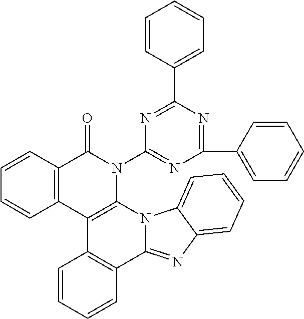 [SMILES: O=c1c2ccccc2c2c3ccccc3c3nc4ccccc4n3c2n1-c1nc(-c2ccccc2)nc(-c2ccccc2)n1] | 55 |
| 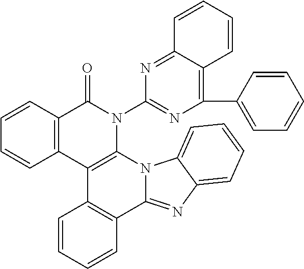 [SMILES: O=c1c2ccccc2c2c3ccccc3c3nc4ccccc4n3c2n1-c1nc(-c2ccccc2)c2ccccc2n1] | 56 |
| 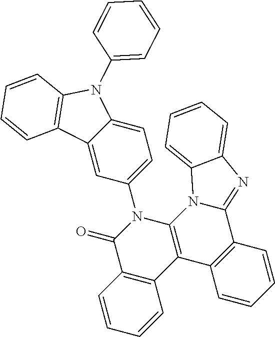 [SMILES: O=c1c2ccccc2c2c3ccccc3c3nc4ccccc4n3c2n1-c1ccc2c(c1)c1ccccc1n2-c1ccccc1] | 57 |
| 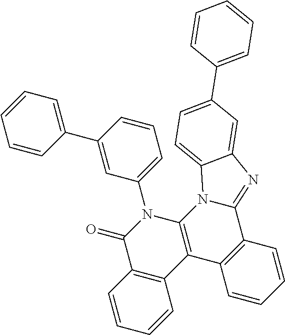 [SMILES: O=c1c2ccccc2c2c3ccccc3c3nc4cc(-c5ccccc5)ccc4n3c2n1-c1cccc(-c2ccccc2)c1] | 58 |
| 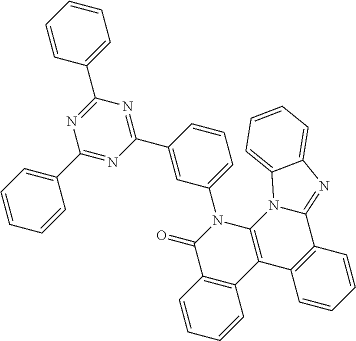 [SMILES: O=c1c2ccccc2c2c3ccccc3c3nc4ccccc4n3c2n1-c1cccc(-c2nc(-c3ccccc3)nc(-c3ccccc3)n2)c1] | 59 |
| 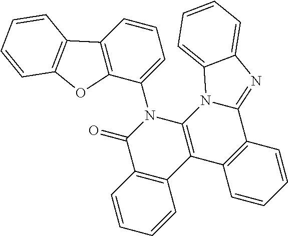 [SMILES: O=c1c2ccccc2c2c3ccccc3c3nc4ccccc4n3c2n1-c1cccc2c1oc1ccccc12] | 60 |
| 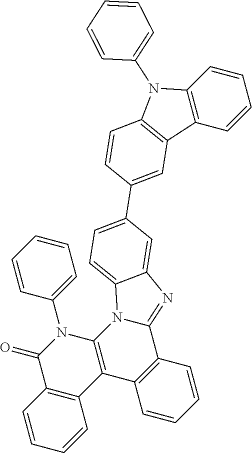 [SMILES: O=c1c2ccccc2c2c3ccccc3c3nc4cc(-c5ccc6c(c5)c5ccccc5n6-c5ccccc5)ccc4n3c2n1-c1ccccc1] | 61 |
| 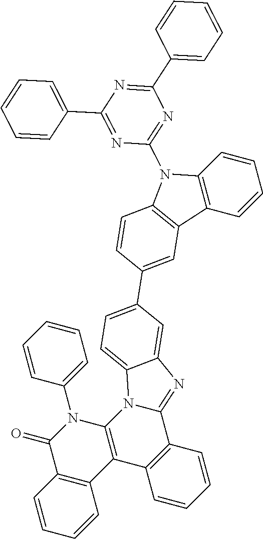 [SMILES: O=c1c2ccccc2c2c3ccccc3c3nc4cc(-c5ccc6c(c5)c5ccccc5n6-c5nc(-c6ccccc6)nc(-c6ccccc6)n5)ccc4n3c2n1-c1ccccc1] | 62 |
| 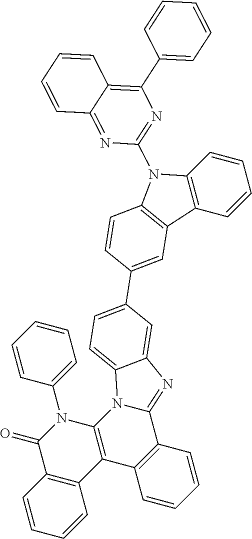 [SMILES: O=c1c2ccccc2c2c3ccccc3c3nc4cc(-c5ccc6c(c5)c5ccccc5n6-c5nc(-c6ccccc6)c6ccccc6n5)ccc4n3c2n1-c1ccccc1] | 63 |
| 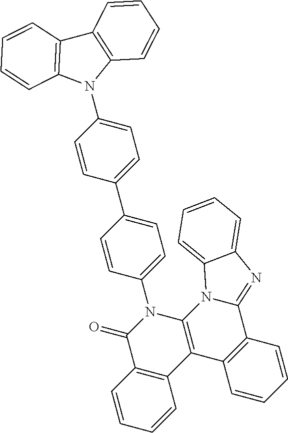 [SMILES: O=c1c2ccccc2c2c3ccccc3c3nc4ccccc4n3c2n1-c1ccc(-c2ccc(-n3c4ccccc4c4ccccc43)cc2)cc1] | 64 |
| 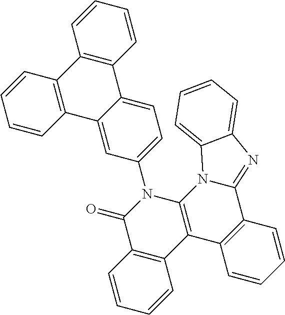 [SMILES: O=c1c2ccccc2c2c3ccccc3c3nc4ccccc4n3c2n1-c1ccc2c3ccccc3c3ccccc3c2c1] | 65 |
| 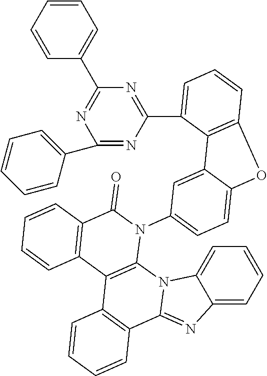 [SMILES: O=c1c2ccccc2c2c3ccccc3c3nc4ccccc4n3c2n1-c1ccc2oc3cccc(-c4nc(-c5ccccc5)nc(-c5ccccc5)n4)c3c2c1] | 66 |
| 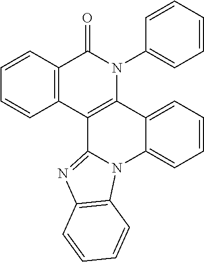 [SMILES: O=c1c2ccccc2c2c(c3ccccc3n3c4ccccc4nc23)n1-c1ccccc1] | 67 |
| 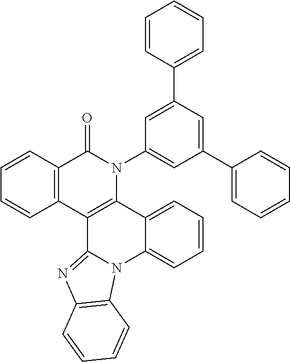 [SMILES: O=c1c2ccccc2c2c(c3ccccc3n3c4ccccc4nc23)n1-c1cc(-c2ccccc2)cc(-c2ccccc2)c1] | 68 |
| 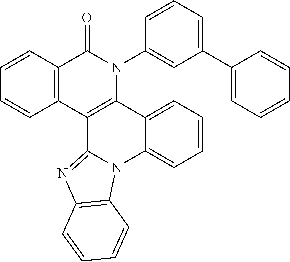 [SMILES: O=c1c2ccccc2c2c(c3ccccc3n3c4ccccc4nc23)n1-c1cccc(-c2ccccc2)c1] | 69 |
| 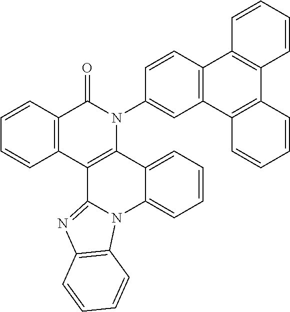 [SMILES: O=c1c2ccccc2c2c(c3ccccc3n3c4ccccc4nc23)n1-c1ccc2c3ccccc3c3ccccc3c2c1] | 70 |
| 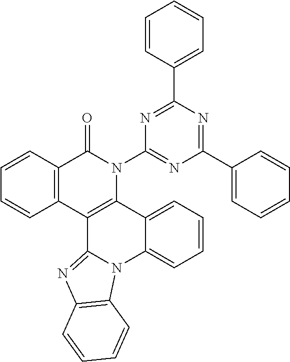 [SMILES: O=c1c2ccccc2c2c(c3ccccc3n3c4ccccc4nc23)n1-c1nc(-c2ccccc2)nc(-c2ccccc2)n1] | 71 |
| 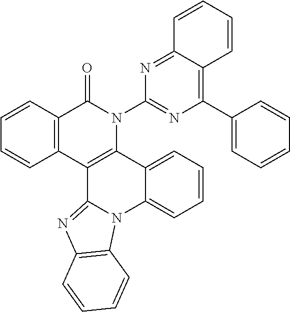 [SMILES: O=c1c2ccccc2c2c(c3ccccc3n3c4ccccc4nc23)n1-c1nc(-c2ccccc2)c2ccccc2n1] | 72 |
| 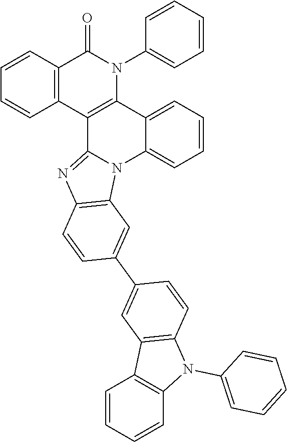 [SMILES: O=c1c2ccccc2c2c(c3ccccc3n3c4cc(-c5ccc6c(c5)c5ccccc5n6-c5ccccc5)ccc4nc23)n1-c1ccccc1] | 73 |
| 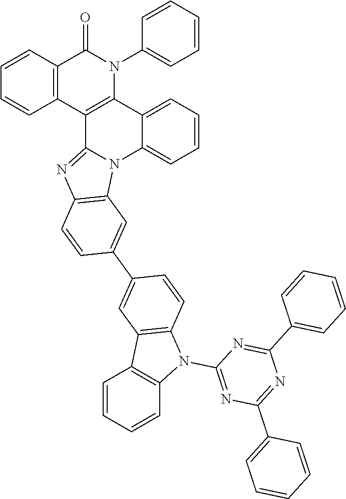 [SMILES: O=c1c2ccccc2c2c(c3ccccc3n3c4cc(-c5ccc6c(c5)c5ccccc5n6-c5nc(-c6ccccc6)nc(-c6ccccc6)n5)ccc4nc23)n1-c1ccccc1] | 74 |
| 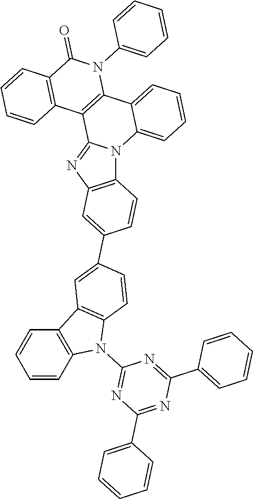 [SMILES: O=c1c2ccccc2c2c(c3ccccc3n3c4ccc(-c5ccc6c(c5)c5ccccc5n6-c5nc(-c6ccccc6)nc(-c6ccccc6)n5)cc4nc23)n1-c1ccccc1] | 75 |
| 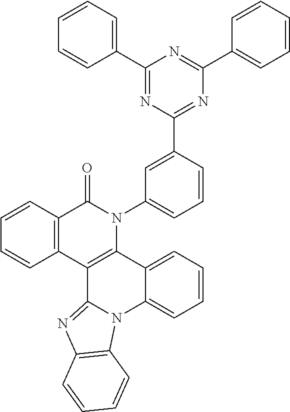 [SMILES: O=c1c2ccccc2c2c(c3ccccc3n3c4ccccc4nc23)n1-c1cccc(-c2nc(-c3ccccc3)nc(-c3ccccc3)n2)c1] | 76 |
| 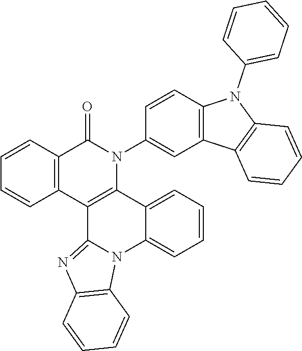 [SMILES: O=c1c2ccccc2c2c(c3ccccc3n3c4ccccc4nc23)n1-c1ccc2c(c1)c1ccccc1n2-c1ccccc1] | 77 |
| 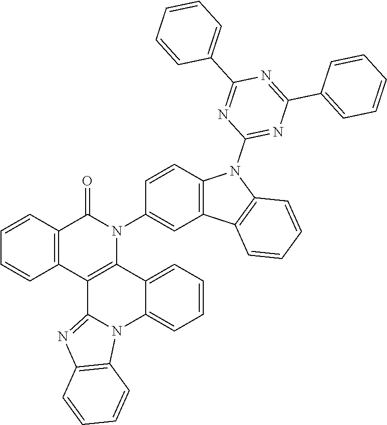 [SMILES: O=c1c2ccccc2c2c(c3ccccc3n3c4ccccc4nc23)n1-c1ccc2c(c1)c1ccccc1n2-c1nc(-c2ccccc2)nc(-c2ccccc2)n1] | 78 |
| 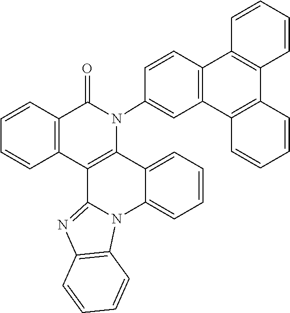 [SMILES: O=c1c2ccccc2c2c(c3ccccc3n3c4ccccc4nc23)n1-c1ccc2c3ccccc3c3ccccc3c2c1] | 79 |
| 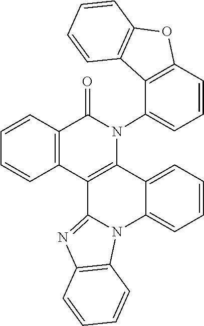 [SMILES: O=c1c2ccccc2c2c(c3ccccc3n3c4ccccc4nc23)n1-c1cccc2oc3ccccc3c12] | 80 |
| 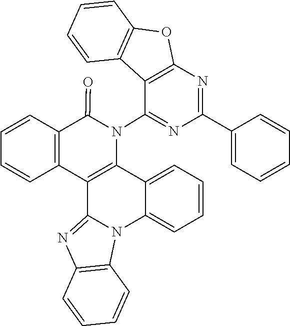 [SMILES: O=c1c2ccccc2c2c(c3ccccc3n3c4ccccc4nc23)n1-c1nc(-c2ccccc2)nc2oc3ccccc3c12] | 81 |
| 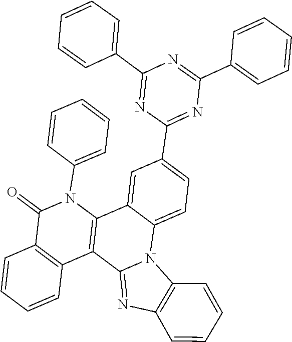 [SMILES: O=c1c2ccccc2c2c(c3cc(-c4nc(-c5ccccc5)nc(-c5ccccc5)n4)ccc3n3c4ccccc4nc23)n1-c1ccccc1] | 82 |
| 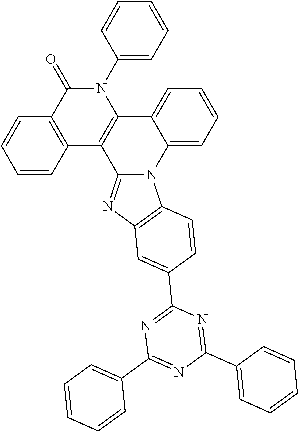 [SMILES: O=c1c2ccccc2c2c(c3ccccc3n3c4ccc(-c5nc(-c6ccccc6)nc(-c6ccccc6)n5)cc4nc23)n1-c1ccccc1] | 83 |
| 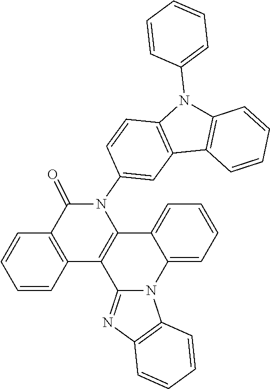 [SMILES: O=c1c2ccccc2c2c(c3ccccc3n3c4ccccc4nc23)n1-c1ccc2c(c1)c1ccccc1n2-c1ccccc1] | 84 |
| 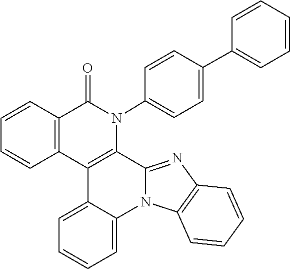 [SMILES: O=c1c2ccccc2c2c3ccccc3n3c4ccccc4nc3c2n1-c1ccc(-c2ccccc2)cc1] | 85 |
| 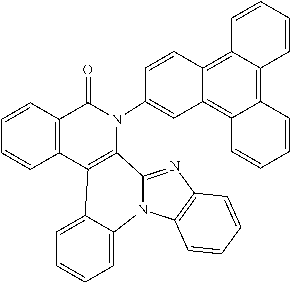 [SMILES: O=c1c2ccccc2c2c3ccccc3n3c4ccccc4nc3c2n1-c1ccc2c3ccccc3c3ccccc3c2c1] | 86 |
| 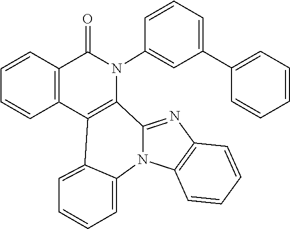 [SMILES: O=c1c2ccccc2c2c3ccccc3n3c4ccccc4nc3c2n1-c1cccc(-c2ccccc2)c1] | 87 |
| 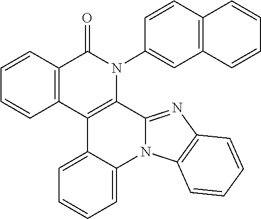 [SMILES: O=c1c2ccccc2c2c3ccccc3n3c4ccccc4nc3c2n1-c1ccc2ccccc2c1] | 88 |
| 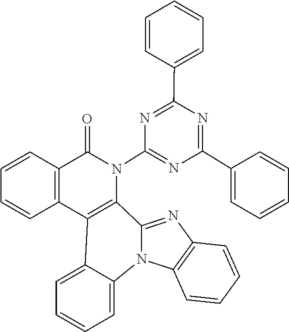 [SMILES: O=c1c2ccccc2c2c3ccccc3n3c4ccccc4nc3c2n1-c1nc(-c2ccccc2)nc(-c2ccccc2)n1] | 89 |
| 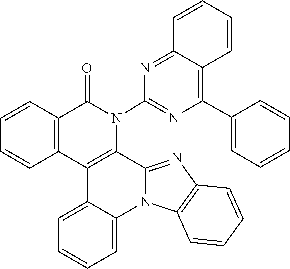 [SMILES: O=c1c2ccccc2c2c3ccccc3n3c4ccccc4nc3c2n1-c1nc(-c2ccccc2)c2ccccc2n1] | 90 |
| 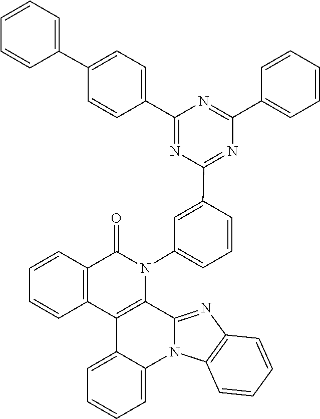 [SMILES: O=c1c2ccccc2c2c3ccccc3n3c4ccccc4nc3c2n1-c1cccc(-c2nc(-c3ccccc3)nc(-c3ccc(-c4ccccc4)cc3)n2)c1] | 91 |
| 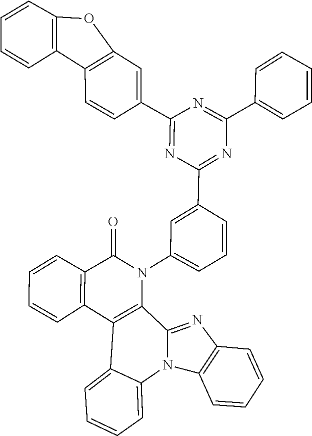 [SMILES: O=c1c2ccccc2c2c3ccccc3n3c4ccccc4nc3c2n1-c1cccc(-c2nc(-c3ccccc3)nc(-c3ccc4c(c3)oc3ccccc34)n2)c1] | 92 |
| 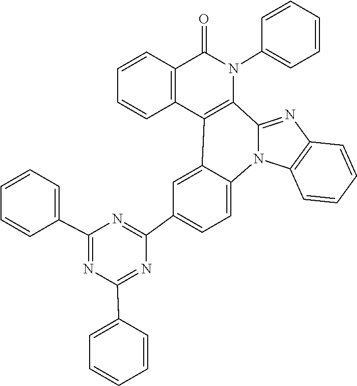 [SMILES: O=c1c2ccccc2c2c3cc(-c4nc(-c5ccccc5)nc(-c5ccccc5)n4)ccc3n3c4ccccc4nc3c2n1-c1ccccc1] | 93 |
| 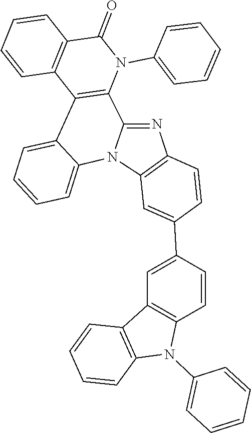 [SMILES: O=c1c2ccccc2c2c3ccccc3n3c4cc(-c5ccc6c(c5)c5ccccc5n6-c5ccccc5)ccc4nc3c2n1-c1ccccc1] | 94 |
| 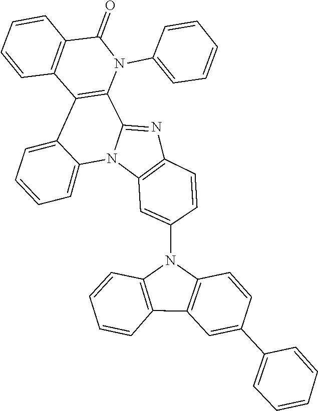 [SMILES: O=c1c2ccccc2c2c3ccccc3n3c4cc(-n5c6ccccc6c6cc(-c7ccccc7)ccc65)ccc4nc3c2n1-c1ccccc1] | 95 |
| 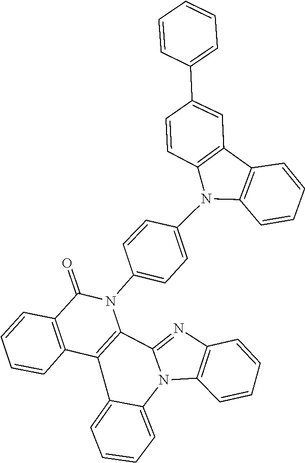 [SMILES: O=c1c2ccccc2c2c3ccccc3n3c4ccccc4nc3c2n1-c1ccc(-n2c3ccccc3c3cc(-c4ccccc4)ccc32)cc1] | 96 |
| 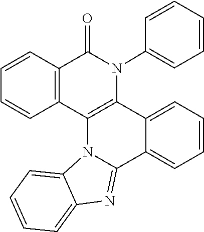 [SMILES: O=c1c2ccccc2c2c(c3ccccc3c3nc4ccccc4n32)n1-c1ccccc1] | 97 |
| 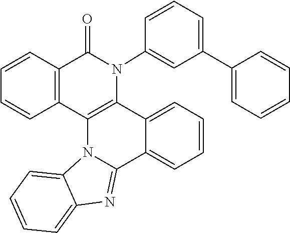 [SMILES: O=c1c2ccccc2c2c(c3ccccc3c3nc4ccccc4n32)n1-c1cccc(-c2ccccc2)c1] | 98 |
| 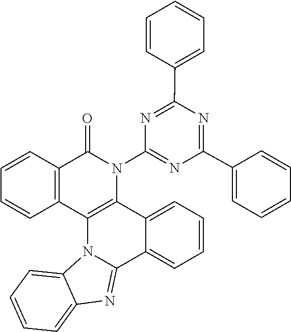 [SMILES: O=c1c2ccccc2c2c(c3ccccc3c3nc4ccccc4n32)n1-c1nc(-c2ccccc2)nc(-c2ccccc2)n1] | 99 |
| 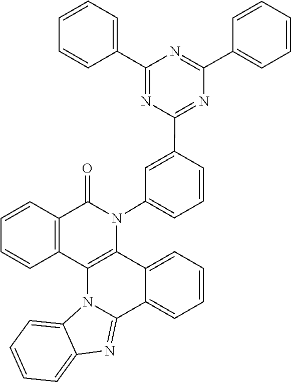 [SMILES: O=c1c2ccccc2c2c(c3ccccc3c3nc4ccccc4n32)n1-c1cccc(-c2nc(-c3ccccc3)nc(-c3ccccc3)n2)c1] | 100 |
| 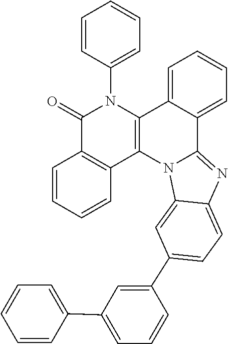 [SMILES: O=c1c2ccccc2c2c(c3ccccc3c3nc4ccc(-c5cccc(-c6ccccc6)c5)cc4n32)n1-c1ccccc1] | 101 |
| 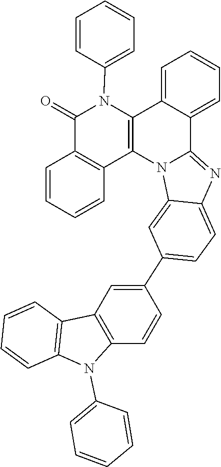 [SMILES: O=c1c2ccccc2c2c(c3ccccc3c3nc4ccc(-c5ccc6c(c5)c5ccccc5n6-c5ccccc5)cc4n32)n1-c1ccccc1] | 102 |
| 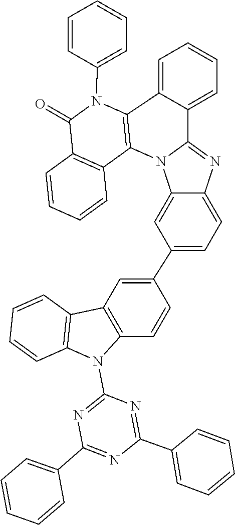 [SMILES: O=c1c2ccccc2c2c(c3ccccc3c3nc4ccc(-c5ccc6c(c5)c5ccccc5n6-c5nc(-c6ccccc6)nc(-c6ccccc6)n5)cc4n32)n1-c1ccccc1] | 103 |
| 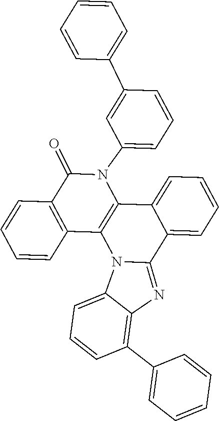 [SMILES: O=c1c2ccccc2c2c(c3ccccc3c3nc4c(-c5ccccc5)cccc4n32)n1-c1cccc(-c2ccccc2)c1] | 104 |
| 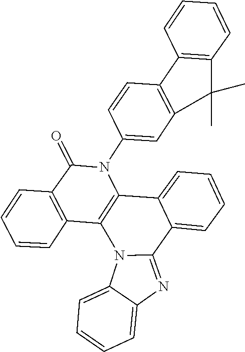 [SMILES: CC1(C)c2ccccc2-c2ccc(-n3c(=O)c4ccccc4c4c3c3ccccc3c3nc5ccccc5n34)cc21] | 105 |
| 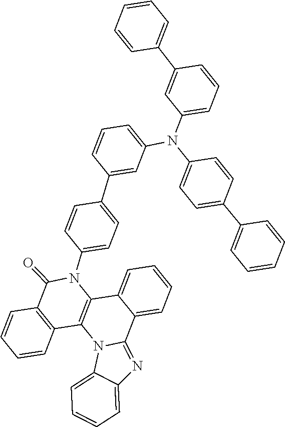 [SMILES: O=c1c2ccccc2c2c(c3ccccc3c3nc4ccccc4n32)n1-c1ccc(-c2cccc(N(c3ccc(-c4ccccc4)cc3)c3cccc(-c4ccccc4)c3)c2)cc1] | 106 |
| 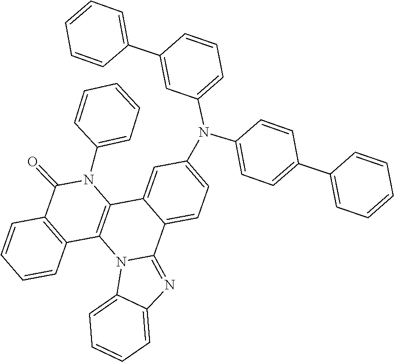 [SMILES: O=c1c2ccccc2c2c(c3cc(N(c4ccc(-c5ccccc5)cc4)c4cccc(-c5ccccc5)c4)ccc3c3nc4ccccc4n32)n1-c1ccccc1] | 107 |
| 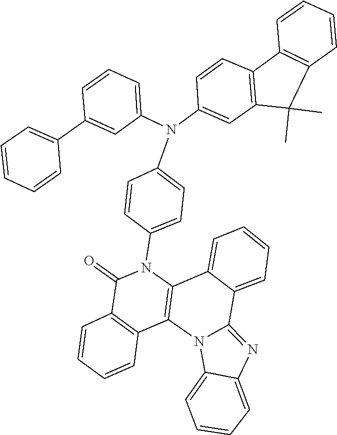 [SMILES: CC1(C)c2ccccc2-c2ccc(N(c3ccc(-n4c(=O)c5ccccc5c5c4c4ccccc4c4nc6ccccc6n45)cc3)c3cccc(-c4ccccc4)c3)cc21] | 108 |
| 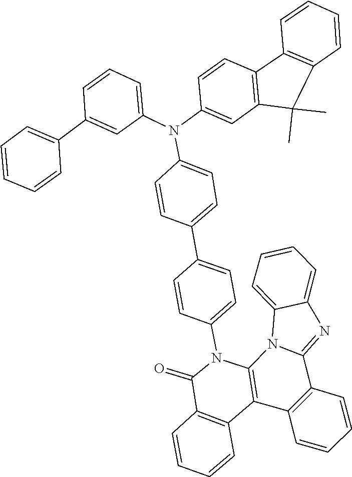 [SMILES: CC1(C)c2ccccc2-c2ccc(N(c3ccc(-c4ccc(-n5c(=O)c6ccccc6c6c7ccccc7c7nc8ccccc8n7c65)cc4)cc3)c3cccc(-c4ccccc4)c3)cc21] | 109 |
| 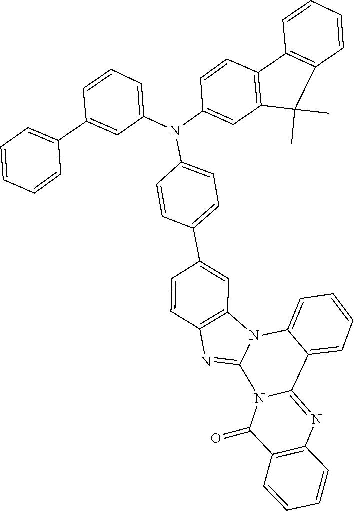 [SMILES: CC1(C)c2ccccc2-c2ccc(N(c3ccc(-c4ccc5nc6n7c(=O)c8ccccc8nc7c7ccccc7n6c5c4)cc3)c3cccc(-c4ccccc4)c3)cc21] | 110 |
| 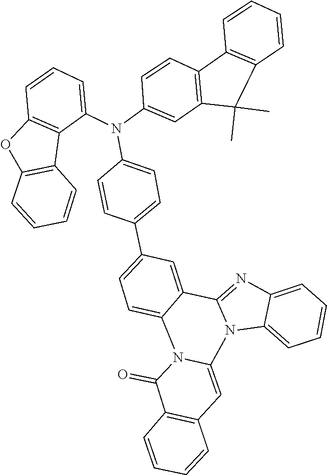 [SMILES: CC1(C)c2ccccc2-c2ccc(N(c3ccc(-c4ccc5c(c4)c4nc6ccccc6n4c4cc6ccccc6c(=O)n54)cc3)c3cccc4oc5ccccc5c34)cc21] | 111 |
| 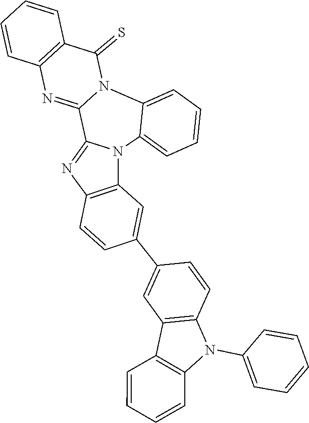 [SMILES: S=c1c2ccccc2nc2c3nc4ccc(-c5ccc6c(c5)c5ccccc5n6-c5ccccc5)cc4n3c3ccccc3n12] | 112 |
| 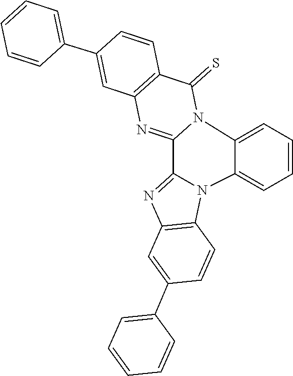 [SMILES: S=c1c2ccc(-c3ccccc3)cc2nc2c3nc4cc(-c5ccccc5)ccc4n3c3ccccc3n12] | 113 |
| 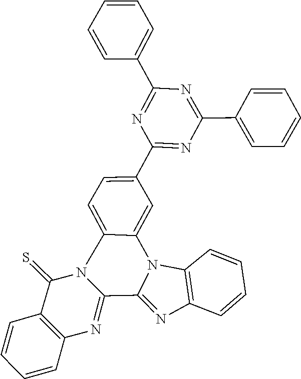 [SMILES: S=c1c2ccccc2nc2c3nc4ccccc4n3c3cc(-c4nc(-c5ccccc5)nc(-c5ccccc5)n4)ccc3n12] | 114 |
| 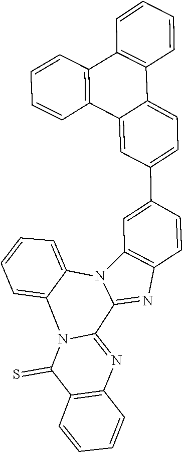 [SMILES: S=c1c2ccccc2nc2c3nc4ccc(-c5ccc6c7ccccc7c7ccccc7c6c5)cc4n3c3ccccc3n12] | 115 |
| 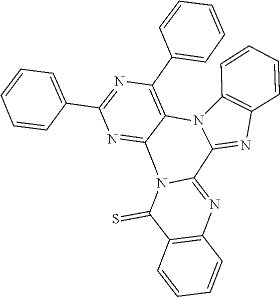 [SMILES: S=c1c2ccccc2nc2c3nc4ccccc4n3c3c(-c4ccccc4)nc(-c4ccccc4)nc3n12] | 116 |
| 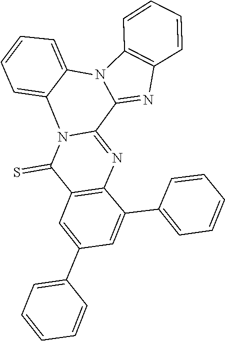 [SMILES: S=c1c2cc(-c3ccccc3)cc(-c3ccccc3)c2nc2c3nc4ccccc4n3c3ccccc3n12] | 117 |
| 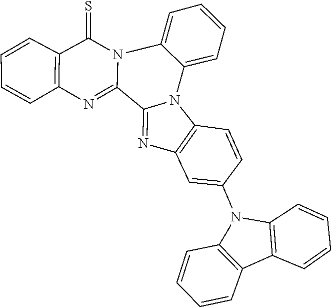 [SMILES: S=c1c2ccccc2nc2c3nc4cc(-n5c6ccccc6c6ccccc65)ccc4n3c3ccccc3n12] | 118 |
| 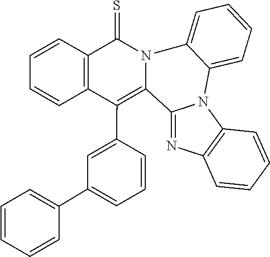 [SMILES: S=c1c2ccccc2c(-c2cccc(-c3ccccc3)c2)c2c3nc4ccccc4n3c3ccccc3n12] | 119 |
| 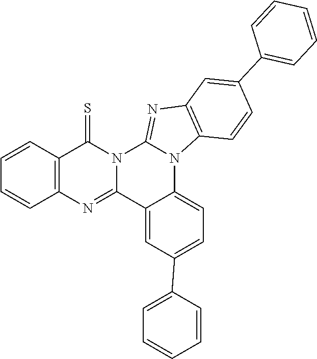 [SMILES: S=c1c2ccccc2nc2c3cc(-c4ccccc4)ccc3n3c4ccc(-c5ccccc5)cc4nc3n12] | 120 |
| 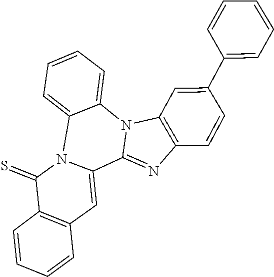 [SMILES: S=c1c2ccccc2cc2c3nc4ccc(-c5ccccc5)cc4n3c3ccccc3n12] | 121 |
| 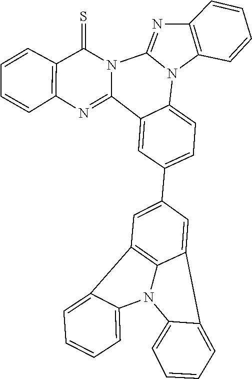 [SMILES: S=c1c2ccccc2nc2c3cc(-c4cc5c6ccccc6n6c7ccccc7c(c4)c56)ccc3n3c4ccccc4nc3n12] | 122 |
| 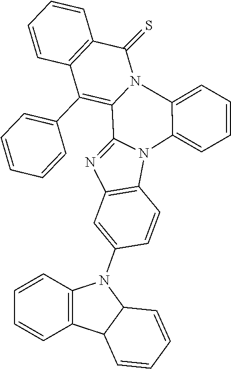 [SMILES: S=c1c2ccccc2c(-c2ccccc2)c2c3nc4cc(N5c6ccccc6C6C=CC=CC65)ccc4n3c3ccccc3n12] | 123 |
| 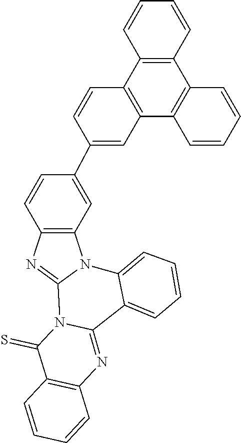 [SMILES: S=c1c2ccccc2nc2c3ccccc3n3c4cc(-c5ccc6c7ccccc7c7ccccc7c6c5)ccc4nc3n12] | 124 |
| 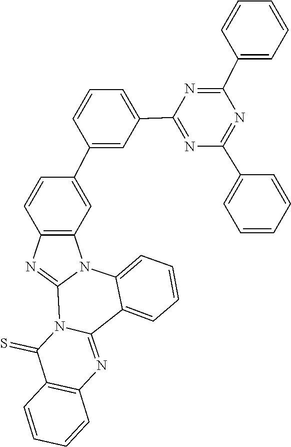 [SMILES: S=c1c2ccccc2nc2c3ccccc3n3c4cc(-c5cccc(-c6nc(-c7ccccc7)nc(-c7ccccc7)n6)c5)ccc4nc3n12] | 125 |
| 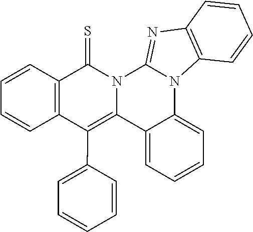 [SMILES: S=c1c2ccccc2c(-c2ccccc2)c2c3ccccc3n3c4ccccc4nc3n12] | 126 |
| 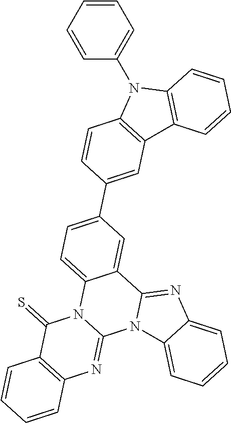 [SMILES: S=c1c2ccccc2nc2n1c1ccc(-c3ccc4c(c3)c3ccccc3n4-c3ccccc3)cc1c1nc3ccccc3n12] | 127 |
| 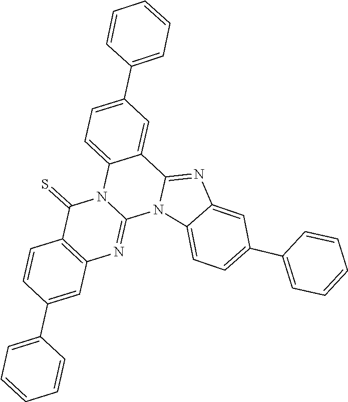 [SMILES: S=c1c2ccc(-c3ccccc3)cc2nc2n1c1ccc(-c3ccccc3)cc1c1nc3cc(-c4ccccc4)ccc3n12] | 128 |
| 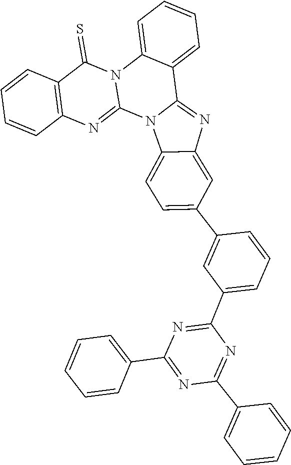 [SMILES: S=c1c2ccccc2nc2n1c1ccccc1c1nc3cc(-c4cccc(-c5nc(-c6ccccc6)nc(-c6ccccc6)n5)c4)ccc3n12] | 129 |
| 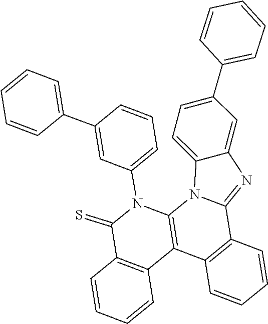 [SMILES: S=c1c2ccccc2c2c3ccccc3c3nc4cc(-c5ccccc5)ccc4n3c2n1-c1cccc(-c2ccccc2)c1] | 130 |
| 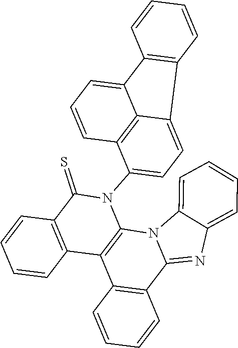 [SMILES: S=c1c2ccccc2c2c3ccccc3c3nc4ccccc4n3c2n1-c1ccc2c3c(cccc13)-c1ccccc1-2] | 131 |
| 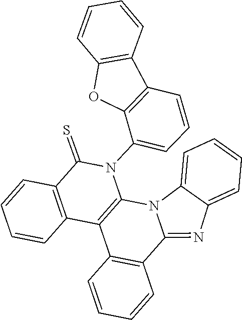 [SMILES: S=c1c2ccccc2c2c3ccccc3c3nc4ccccc4n3c2n1-c1cccc2c1oc1ccccc12] | 132 |
| 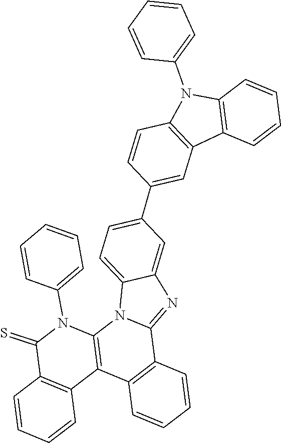 [SMILES: S=c1c2ccccc2c2c3ccccc3c3nc4cc(-c5ccc6c(c5)c5ccccc5n6-c5ccccc5)ccc4n3c2n1-c1ccccc1] | 133 |
| 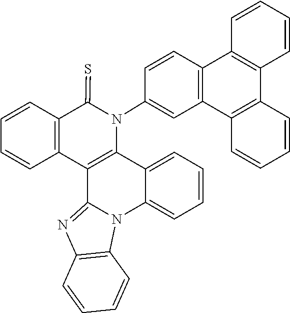 [SMILES: S=c1c2ccccc2c2c(c3ccccc3n3c4ccccc4nc23)n1-c1ccc2c3ccccc3c3ccccc3c2c1] | 134 |
| 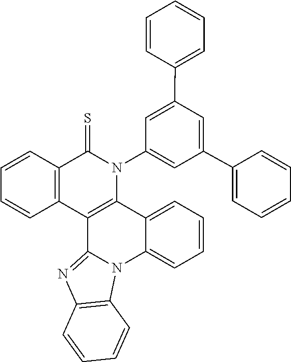 [SMILES: S=c1c2ccccc2c2c(c3ccccc3n3c4ccccc4nc23)n1-c1cc(-c2ccccc2)cc(-c2ccccc2)c1] | 135 |
| 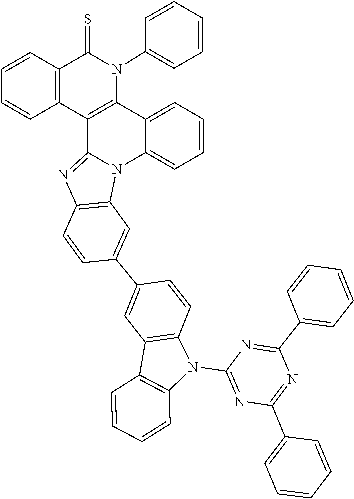 [SMILES: S=c1c2ccccc2c2c(c3ccccc3n3c4cc(-c5ccc6c(c5)c5ccccc5n6-c5nc(-c6ccccc6)nc(-c6ccccc6)n5)ccc4nc23)n1-c1ccccc1] | 136 |
| 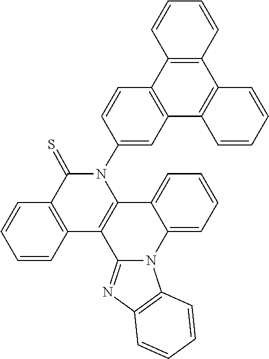 [SMILES: S=c1c2ccccc2c2c(c3ccccc3n3c4ccccc4nc23)n1-c1ccc2c3ccccc3c3ccccc3c2c1] | 137 |
| 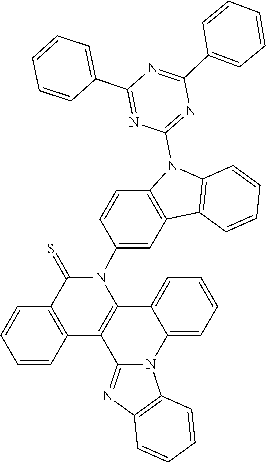 [SMILES: S=c1c2ccccc2c2c(c3ccccc3n3c4ccccc4nc23)n1-c1ccc2c(c1)c1ccccc1n2-c1nc(-c2ccccc2)nc(-c2ccccc2)n1] | 138 |
| 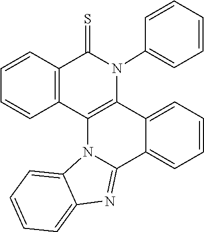 [SMILES: S=c1c2ccccc2c2c(c3ccccc3c3nc4ccccc4n32)n1-c1ccccc1] | 139 |
| 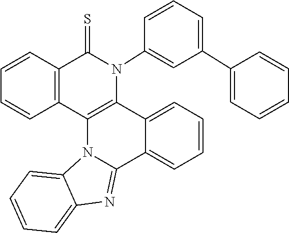 [SMILES: S=c1c2ccccc2c2c(c3ccccc3c3nc4ccccc4n32)n1-c1cccc(-c2ccccc2)c1] | 140 |
| 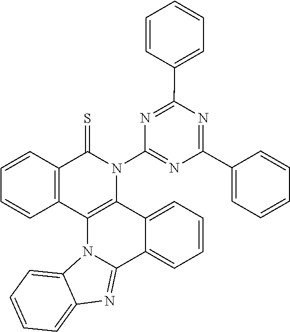 [SMILES: S=c1c2ccccc2c2c(c3ccccc3c3nc4ccccc4n32)n1-c1nc(-c2ccccc2)nc(-c2ccccc2)n1] | 141 |
| 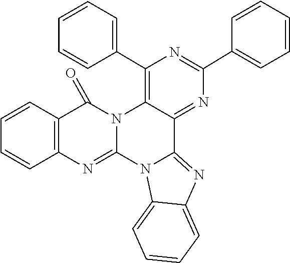 [SMILES: O=c1c2ccccc2nc2n1c1c(-c3ccccc3)nc(-c3ccccc3)nc1c1nc3ccccc3n12] | 142 |
| 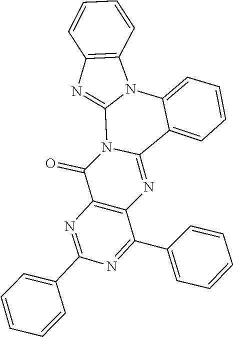 [SMILES: O=c1c2nc(-c3ccccc3)nc(-c3ccccc3)c2nc2c3ccccc3n3c4ccccc4nc3n12] | 143 |
| 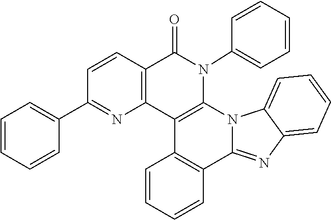 [SMILES: O=c1c2ccc(-c3ccccc3)nc2c2c3ccccc3c3nc4ccccc4n3c2n1-c1ccccc1] | 144 |
| 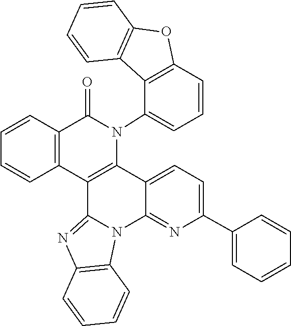 [SMILES: O=c1c2ccccc2c2c(c3ccc(-c4ccccc4)nc3n3c4ccccc4nc23)n1-c1cccc2oc3ccccc3c12] | 145 |
| 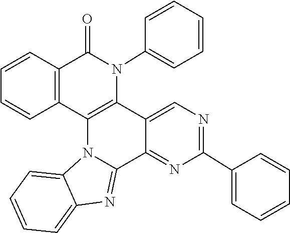 [SMILES: O=c1c2ccccc2c2c(c3cnc(-c4ccccc4)nc3c3nc4ccccc4n32)n1-c1ccccc1] | 146 |
| 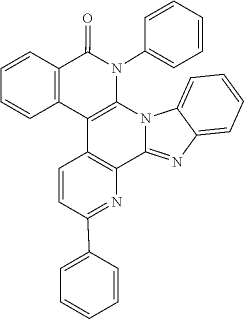 [SMILES: O=c1c2ccccc2c2c3ccc(-c4ccccc4)nc3c3nc4ccccc4n3c2n1-c1ccccc1] | 147 |
| 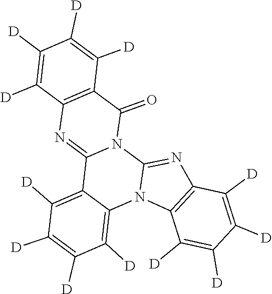 [SMILES: [2H]c1c([2H])c([2H])c2c(=O)n3c(nc2c1[2H])c1c([2H])c([2H])c([2H])c([2H])c1n1c2c([2H])c([2H])c([2H])c([2H])c2nc31] | 148 |
| 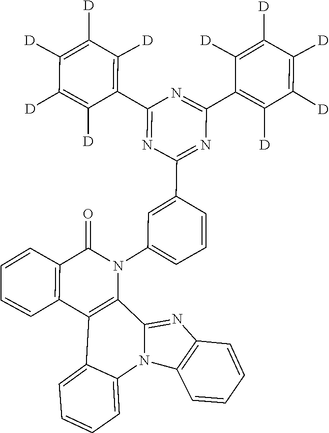 [SMILES: [2H]c1c([2H])c([2H])c(-c2nc(-c3cccc(-n4c(=O)c5ccccc5c5c6ccccc6n6c7ccccc7nc6c54)c3)nc(-c3c([2H])c([2H])c([2H])c([2H])c3[2H])n2)c([2H])c1[2H]] | 149 |
| 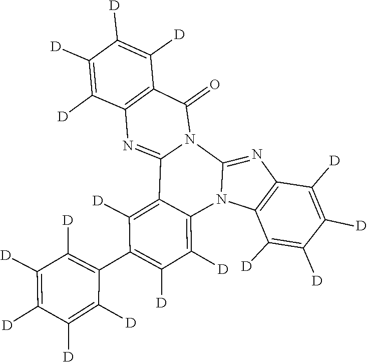 [SMILES: [2H]c1c([2H])c([2H])c(-c2c([2H])c([2H])c3c(c2[2H])c2nc4c([2H])c([2H])c([2H])c([2H])c4c(=O)n2c2nc4c([2H])c([2H])c([2H])c([2H])c4n32)c([2H])c1[2H]] | 150 |
| 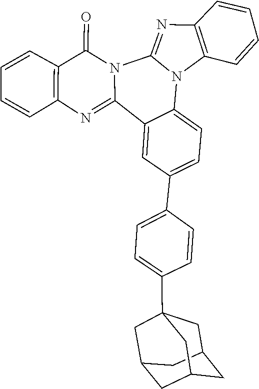 [SMILES: O=c1c2ccccc2nc2c3cc(-c4ccc(C56CC7CC(CC(C7)C5)C6)cc4)ccc3n3c4ccccc4nc3n12] | 151 |
| 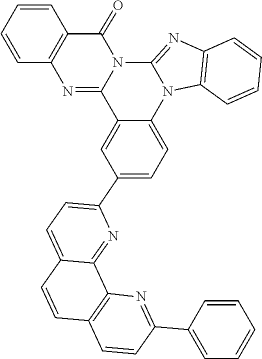 [SMILES: O=c1c2ccccc2nc2c3cc(-c4ccc5ccc6ccc(-c7ccccc7)nc6c5n4)ccc3n3c4ccccc4nc3n12] | 152 |
| 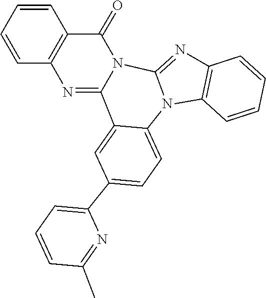 [SMILES: Cc1cccc(-c2ccc3c(c2)c2nc4ccccc4c(=O)n2c2nc4ccccc4n32)n1] | 153 |
| 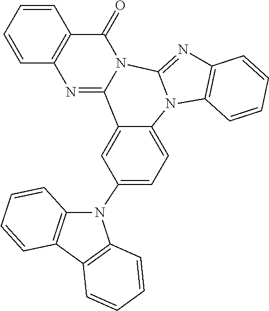 [SMILES: O=c1c2ccccc2nc2c3cc(-n4c5ccccc5c5ccccc54)ccc3n3c4ccccc4nc3n12] | 154 |
| 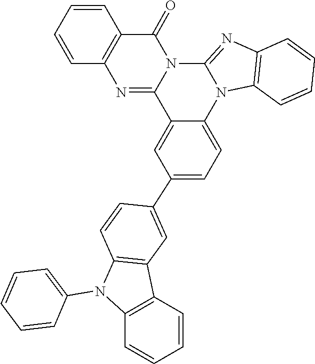 [SMILES: O=c1c2ccccc2nc2c3cc(-c4ccc5c(c4)c4ccccc4n5-c4ccccc4)ccc3n3c4ccccc4nc3n12] | 155 |
| 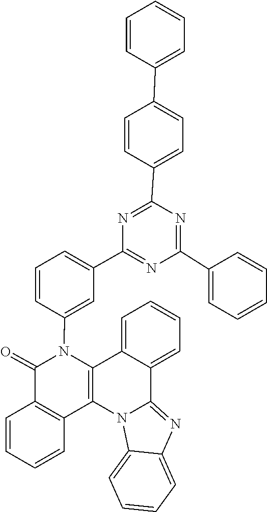 [SMILES: O=c1c2ccccc2c2c(c3ccccc3c3nc4ccccc4n32)n1-c1cccc(-c2nc(-c3ccccc3)nc(-c3ccc(-c4ccccc4)cc3)n2)c1] | 156 |

The base structure of the compounds of the invention can be prepared by the routes outlined in the schemes which follow. The individual synthesis steps, for example C—C coupling reactions according to Suzuki, C—N coupling reactions according to Buchwald or Hartwig-Buchwald or cyclization reactions, are known in principle to those skilled in the art. Further information relating to the synthesis of the compounds of the invention can be found in the synthesis examples. Possible syntheses of the base structure are shown in schemes 1 to 7. These can be effected by known reactions, as detailed by way of example in CN 109535200, WO 2009/086264, WO 2010/059773, WO 2013/132253, KR 2018041482, WO 2018/070836, and in the following publications:

Chem Med Chem (2009), 4(5), 866-876; European Journal of Medicinal Chemistry, 157, 1361-1375; 2018; European Journal of Medicinal Chemistry, 162, 586-601; 2019; Journal of Heterocyclic Chemistry, 56(7), 2046-2051; 2019; Journal of Catalysis, 373, 93-102; 2019; Journal of Organic Chemistry (2010), 75(7), 2302-2308; Journal of the Chemical Society [Section] C: Organic (1971), (7), 1227-31; Journal of the Serbian Chemical Society (1987), 52(11), 633-9; Journal of the Serbian Chemical Society (1989), 54(4), 179-87; Justus Liebigs Annalen der Chemie, 729, 97-105; 1969; Letters in Organic Chemistry, 16(11), 898-905; 2019; Medicinal Chemistry Research, 25(6), 1125-1139; 2016; Organic & Biomolecular Chemistry, 17(18), 4465-4469; 2019; Organic & Biomolecular chemistry 11(45), 7966-7977; 2013; Pharmazie, 35(5-6), 293-6; 1980; Synthesis, (8), 1343-1350; 2006; Synthesis, (16), 2794-2798; 2010; Tetrahedron Letters, 55(16), 2742-2744; 2014; Tetrahedron Letters, 41(31), 5857-5860; 2000 and Tetrahedron (2012), 68(1), 250-261. Scheme 8 shows various further ways of derivatizing the base structure.

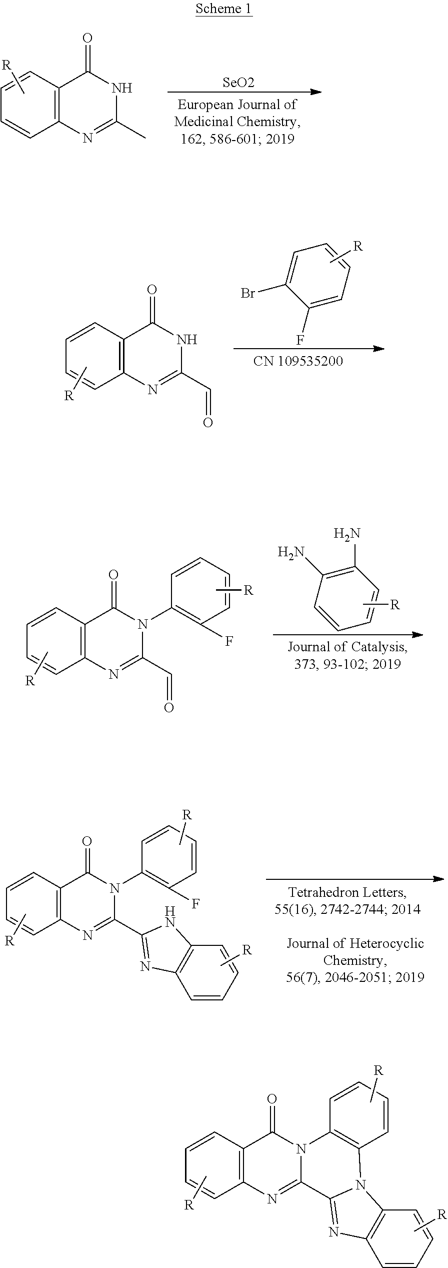

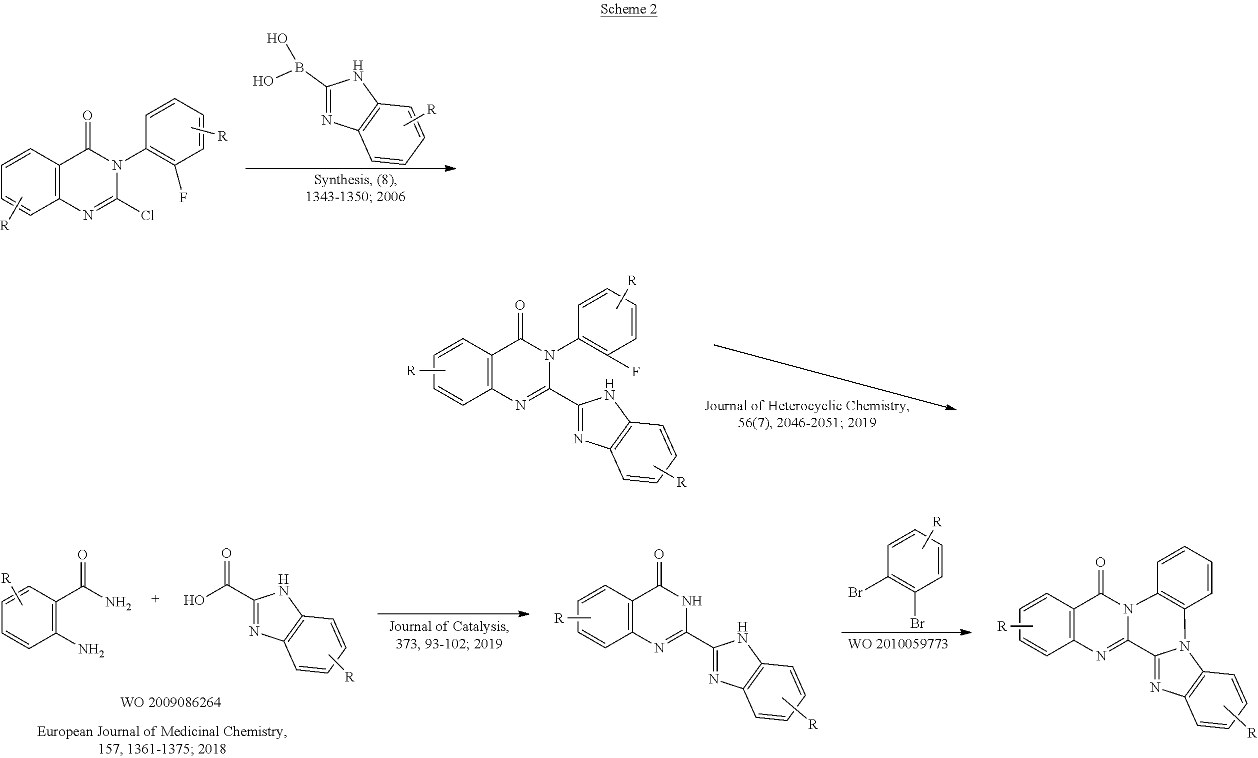

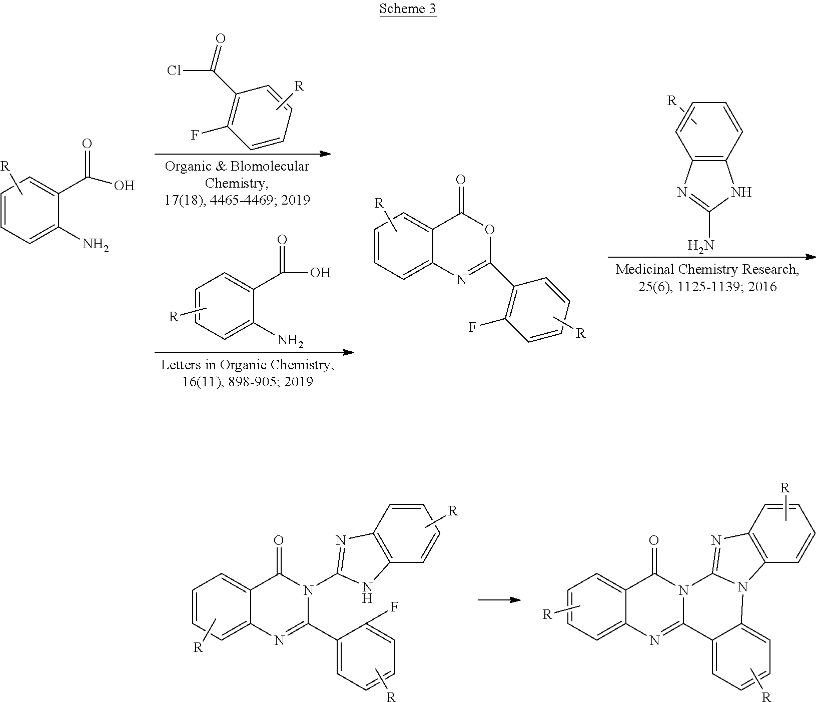

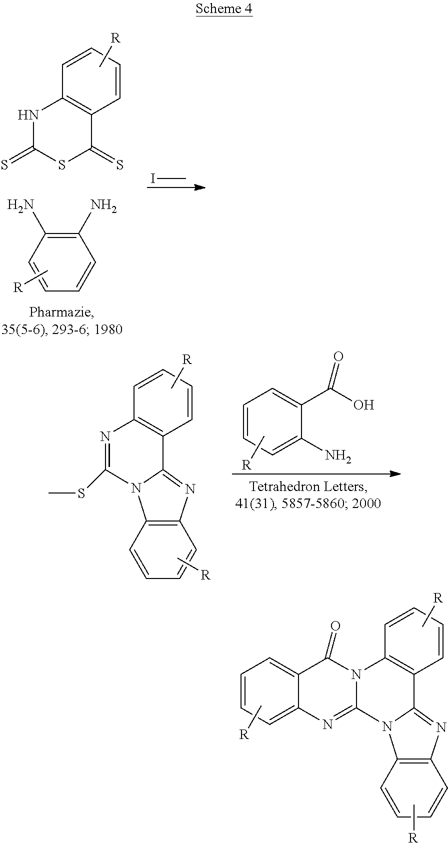

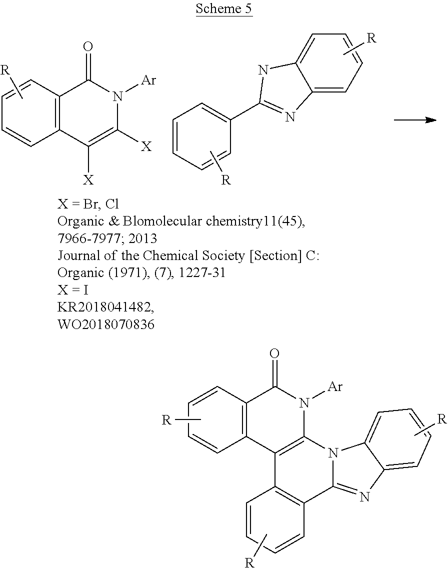

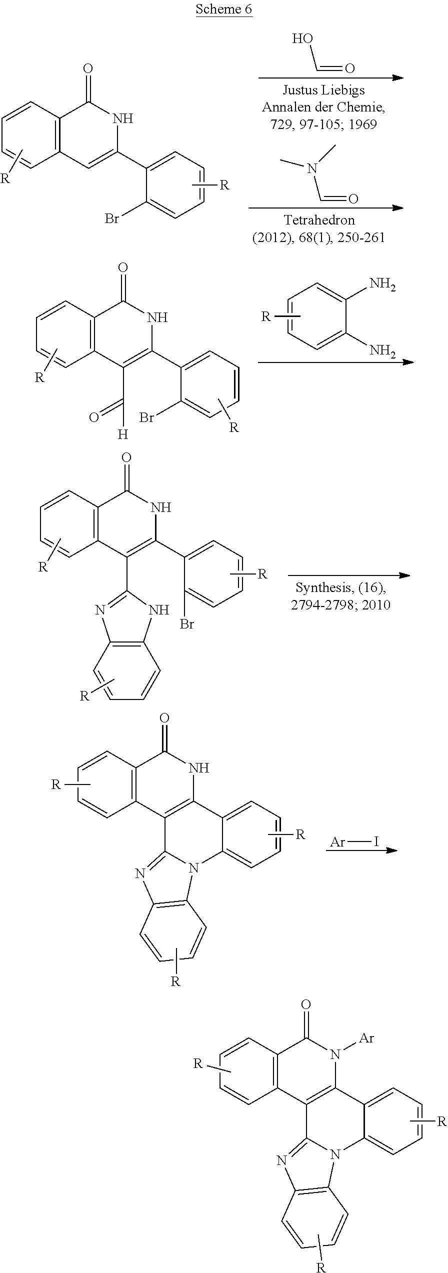

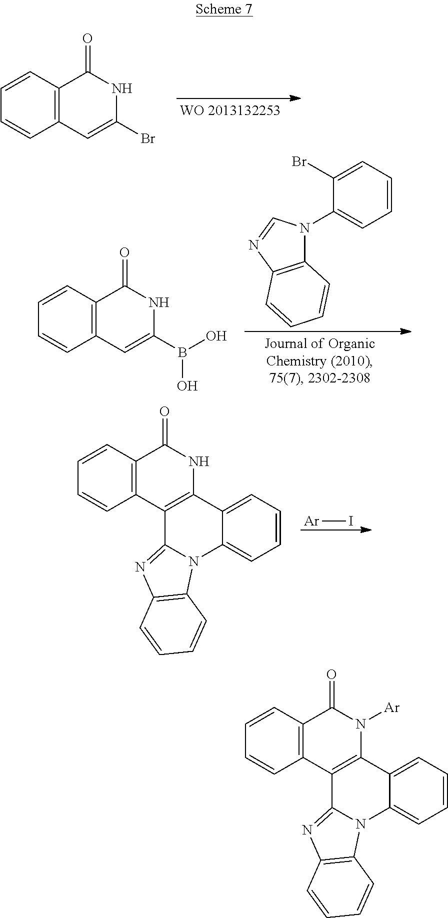

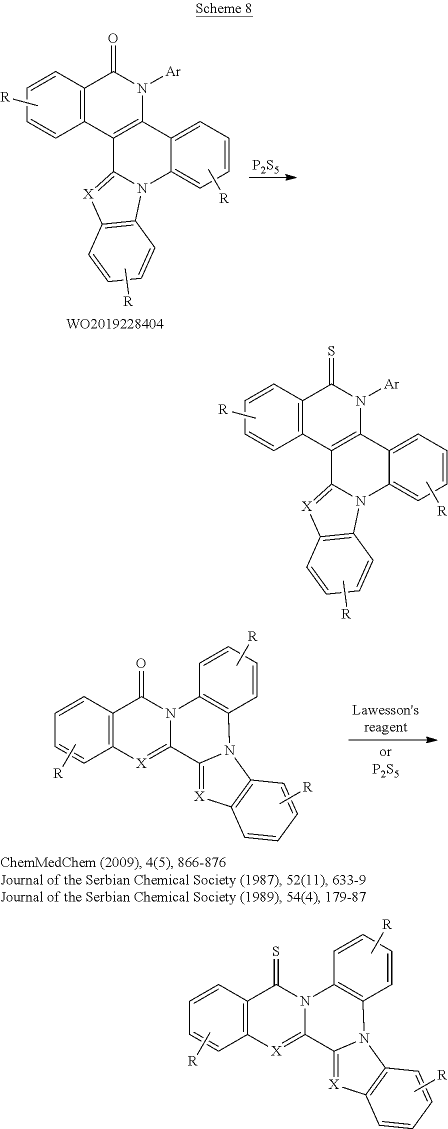

The definition of the symbols used in schemes 1 to 8 corresponds essentially to that which was defined for structural elements (A), (B) and (C), dispensing with numbering and complete representation of all symbols for reasons of clarity. In addition, for reasons of clarity, the use of the symbol X for representation of possible nitrogen atoms in the heteroaromatic rings has been dispensed with in many cases, as shown in particular for structural elements (A), (B) and (C) and in formulae (I-1) to (I-7) and/or (II-1) to (II-30) by the symbols X¹, X² and X³. These details should therefore be understood by way of illustration; the person skilled in the art will be capable of applying syntheses set out above and hereinafter, especially in the examples, to compounds in which one or more of the symbols X¹, X² and X³ are nitrogen.

The present invention therefore further provides a process for preparing a compound of the invention, wherein a nitrogen-containing aromatic or heteroaromatic compound is reacted in a ring-forming reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. The further compound may also be polymeric.

The present invention further provides for the use of a compound containing at least one compound comprising at least one structure having at least three mutually fused structural elements of the formulae (A′), (B′) and (C′), preferably a compound consisting of three mutually fused structural elements of the formulae (A′), (B′) and (C′):

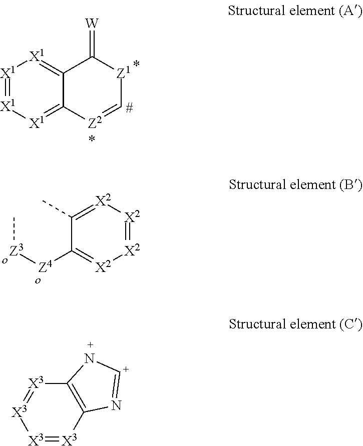

where structural element (A′) is fused to structural element (B′) and structural element (B′) is fused to structural element (C′); where structural element (B′) binds to structural element (A′) via the bonds shown by dotted lines, where there is one bond via the binding site marked # and one bond via a binding site marked *; where structural element (B′) is fused to structural element (C′) via the atoms marked o and +, and the respectively marked atoms are shared by structural elements (B′) and (C′), and
the symbols and indices used are as follows:
W is O or S, preferably O;
Z¹ is NAr, or N if structural element (A′) is fused to structural element (B′) via Z¹;
Z² is X, or C if structural element (A′) is fused to structural element (B′) via Z²;
Z³, Z⁴ is N or C, where one of the Z³, Z⁴ radicals is N and one of the Z³, Z⁴ radicals is C;
X is N or CR, preferably N;
X¹ is the same or different at each instance and is N or CR¹, preferably CR¹, with the proviso that not more than two of the X¹ groups in one cycle are N;
X² is the same or different at each instance and is N or CR², with the proviso that not more than two of the X² groups in one cycle are N;
X³ is the same or different at each instance and is N or CR³, preferably CR³, with the proviso that not more than two of the X³ groups in one cycle are N;
Ar is the same or different at each instance and is an aromatic or heteroaromatic ring system which has 5 to 40 aromatic ring atoms and may be substituted by one or more R radicals, preferably an aryl group which has 6 to 30 aromatic ring atoms or a heteroaryl group which has 5 to 14 aromatic ring atoms and may be substituted by one or more R radicals;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for one R radical together with a further radical, preferably an R¹, R², R³ group, to form an aliphatic, heteroaliphatic, aromatic or heteroaromatic ring system, preferably an aliphatic, heteroaliphatic or heteroaromatic ring system; more preferably, the R radical does not form any such ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals or one R¹ radical together with a further radical, preferably an R, R², R³ group, to form an aromatic, heteroaromatic, aliphatic or heteroaliphatic ring system, preferably an aliphatic, heteroaliphatic or heteroaromatic ring system; more preferably, the R¹ radicals do not form any such ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, preferably an R, R¹, R³ group, to form an aromatic, heteroaromatic, aliphatic or heteroaliphatic ring system, preferably an aliphatic, heteroaliphatic or heteroaromatic ring system; more preferably, the R² radicals do not form any such ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, preferably an R, R¹, R² group, to form an aromatic, heteroaromatic, aliphatic or heteroaliphatic ring system, preferably an aliphatic, heteroaliphatic or heteroaromatic ring system; more preferably, the R³ radicals do not form any such ring system;
Ar′ is the same or different at each instance and is an aromatic or heteroaromatic ring system which has 5 to 40 aromatic ring atoms and has R⁴ radicals, preferably an aryl group which has 6 to 30 aromatic ring atoms or a heteroaryl group which has 5 to 14 aromatic ring atoms and has R⁴ radicals;
R⁴ is the same or different at each instance and is H, D, F, Cl, Br, I, R⁵C≡C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system, preferably an aliphatic ring system; more preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
   in an electronic device, especially in an organic electroluminescent device.

The groups defined for structural elements (A′), (B′) and (C′) in many cases correspond to the radicals defined above for structural elements (A), (B) and (C), and so the details, definitions and/or preferences set out above are also applicable to the structural elements (A′), (B′) and (C′). In addition, structures/compounds comprising at least three mutually fused structural elements of the formulae (A′), (B′) and (C′), and preferred embodiments of these, are preferred structures/compounds comprising at least three mutually fused structural elements of the formulae (A), (B) and (C), and so the details given above and hereinafter in this regard are also correspondingly applicable to structures/compounds comprising at least three mutually fused structural elements of the formulae (A′), (B′) and (C′).

The present invention still further provides an electronic device containing at least one compound comprising at least one structure having at least three mutually fused structural elements of the formulae (A′), (B′) and (C′), preferably a compound consisting of three mutually fused structural elements of the formulae (A′), (B′) and (C′):

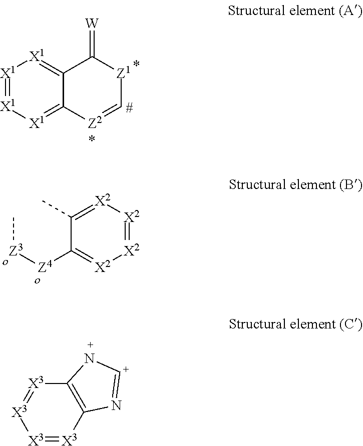

where structural element (A′) is fused to structural element (B′) and structural element (B′) is fused to structural element (C′); where structural element (B′) binds to structural element (A′) via the bonds shown by dotted lines, where there is one bond via the binding site marked # and one bond via a binding site marked *; where structural element (B′) is fused to structural element (C′) via the atoms marked o and +, and the respectively marked atoms are shared by structural elements (B′) and (C′);
where the symbols and indices used have the definitions given above, especially for structural elements (A′), (B′) and (C′);
where the electronic device is preferably an electroluminescent device.

An electronic device in the context of the present invention is a device comprising at least one layer comprising at least one organic compound.

This component may also comprise inorganic materials or else layers formed entirely from inorganic materials.

The electronic device is more preferably selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device containing a compound comprising at least one structure having at least three mutually fused structural elements of the formulae (A′), (B′) and (C′), preferably a compound consisting of three mutually fused structural elements of the formulae (A′), (B′) and (C′), as matrix material for phosphorescent emitters or for emitters that exhibit TADF (thermally activated delayed fluorescence), especially for phosphorescent emitters. In addition, the compound of the invention can also be used in an electron transport layer and/or in a hole blocker layer in an electron blocker layer, preferably in an electron transport layer. More preferably, the compound of the invention is used as matrix material for phosphorescent emitters, especially for red-, orange-, green- or yellow-phosphorescing, preferably red- or green-phosphorescing, emitters in an emitting layer or as electron transport in an electron transport layer, more preferably as matrix material in an emitting layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of a compound comprising at least one structure having at least three mutually fused structural elements of the formulae (A′), (B′) and (C′), or the formulae (A), (B) and (C), or a preferred embodiment thereof as matrix material for a phosphorescent emitter in combination with a further matrix material. Any further matrix material which is used in addition to a compound comprising at least one structure having at least three mutually fused structural elements of the formulae (A′), (B′) and (C′), or the formulae (A), (B) and (C), or a preferred embodiment is also sometimes referred to hereinafter as co-host. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Particularly preferred co-host materials that can be used in combination with a compound comprising at least one structure having at least three mutually fused structural elements of the formulae (A′), (B′) and (C′) or a preferred embodiment thereof are compounds of one of the formulae (H-1), (H-2), (H-3), (H-4) and (H-5):

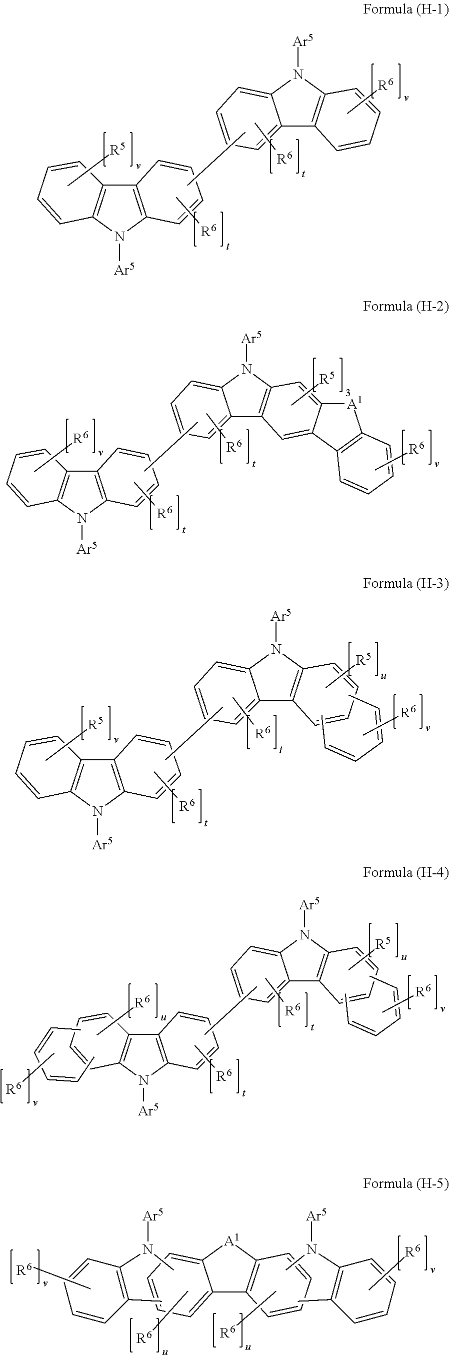

where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, preferably 5 to 40 aromatic ring atoms, and in each case has R⁷ radicals; at the same time, two R⁶ radicals together may also form an aromatic, heteroaromatic, aliphatic or heteroaliphatic ring system; preferably, the R⁶ radicals do not form any such ring system;
Ar″ is the same or different at each instance and is an aromatic or heteroaromatic ring system which has 5 to 40 aromatic ring atoms and has R⁷ radicals;
A¹ is C(R⁷)₂, NR⁷, O or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R⁸)₂, CN, NO₂, OR^{B}, SR^{B}, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R⁸ radicals; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4, preferably 0 or 1 and very preferably O;
t is the same or different at each instance and is 0, 1, 2 or 3, preferably 0 or 1 and very preferably O;
u is the same or different at each instance and is 0, 1 or 2, preferably 0 or 1 and very preferably 0.

The sum total of the indices v, t and u in compounds of the formulae (H-1), (H-2), (H-3), (H-4) or (H-5) is preferably not more than 6, more preferably not more than 4 and especially preferably not more than 2.

In a preferred embodiment of the invention, R⁶ is the same or different at each instance and is selected from the group consisting of H, D, F, CN, NO₂, Si(R⁷)₃, B(OR⁷)₂, a straight-chain alkyl group having 1 to 20 carbon atoms or a branched or cyclic alkyl group having 3 to 20 carbon atoms, where the alkyl group may be substituted in each case by one or more R7 radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, an aromatic or heteroaromatic ring system which has 6 to 30 aromatic ring atoms and may be substituted by one or more R⁷ radicals, and an N(Ar″)₂ group. More preferably, R⁶ is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁷ radicals.

Preferred aromatic or heteroaromatic ring systems represented by the R⁶ or Ar″ groups are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. The structures Ar-1 to Ar-79 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-79 set out above, in relation to the R⁶ and Ar″ radicals, the substituents R⁴ should be replaced by the corresponding R⁷ radicals. The preferences set out above for the R¹, R² and R³ groups are correspondingly applicable to the R⁶ group.

Further suitable R⁶ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40. Further preferences for the Ar², Ar³ and Ar⁴ groups have been set out above and are correspondingly applicable.

It may further be the case that the substituents R⁶ according to the above formulae do not form a fused aromatic or heteroaromatic ring system, preferably any fused ring system, with the ring atoms of the ring system. This includes the formation of a fused ring system with possible substituents R⁷, R⁸ which may be bonded to the R⁶ radicals.

When A¹ is NR⁷, the substituent R⁷ bonded to the nitrogen atom is preferably an aromatic or heteroaromatic ring system which has 5 to 24 aromatic ring atoms and may also be substituted by one or more R⁸ radicals. In a particularly preferred embodiment, this R⁷ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁸ radicals. Preference is given to phenyl, biphenyl, terphenyl and quaterphenyl having bonding patterns as listed above for Ar-1 to Ar-11, where these structures, rather than by R⁴, may be substituted by one or more R⁸ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁸ radicals.

When A¹ is C(R⁷)₂, the substituents R⁷ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁷ is a methyl group or a phenyl group. In this case, the R⁷ radicals together may also form a ring system, which leads to a spiro system.

Preferred aromatic or heteroaromatic ring systems Ar⁵ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals.

The Ar⁵ groups here are more preferably independently selected from the groups of the formulae Ar-1 to Ar-79 set out above, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-79 set out above, in relation to the Ar⁵ radicals, the substituents R⁴ by the corresponding R⁷ radicals.

In a further preferred embodiment of the invention, R⁷ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R⁸ radicals, or an aromatic or heteroaromatic ring system which has 6 to 24 aromatic ring atoms and may be substituted in each case by one or more R⁸ radicals. In a particularly preferred embodiment of the invention, R⁷ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁸ radicals, but is preferably unsubstituted, or an aromatic or heteroaromatic ring system which has 6 to 13 aromatic ring atoms and may be substituted in each case by one or more R⁸ radicals, but is preferably unsubstituted.

In a further preferred embodiment of the invention, R⁸ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

Preferred embodiments of the compounds of the formulae (H-1) and (H-2) are the compounds of the following formulae (H-1a) and (H-2a):

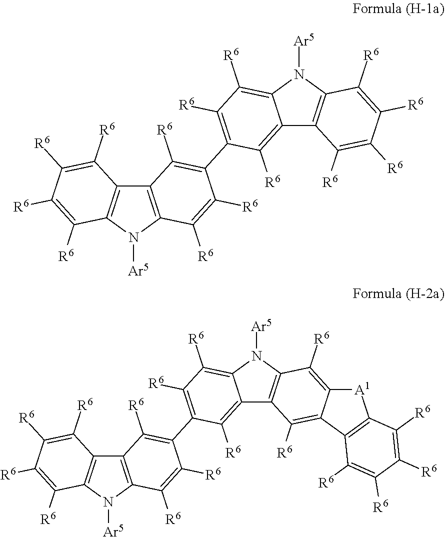

where R⁶, Ar⁵ and A¹ have the definitions given above, especially for formula (H-1) or (H-2). In a preferred embodiment of the invention, A¹ in formula (H-2a) is C(R⁷)₂.

Preferred embodiments of the compounds of the formulae (H-1a) and (H-2a) are the compounds of the following formulae (H-1b) and (H-2b):

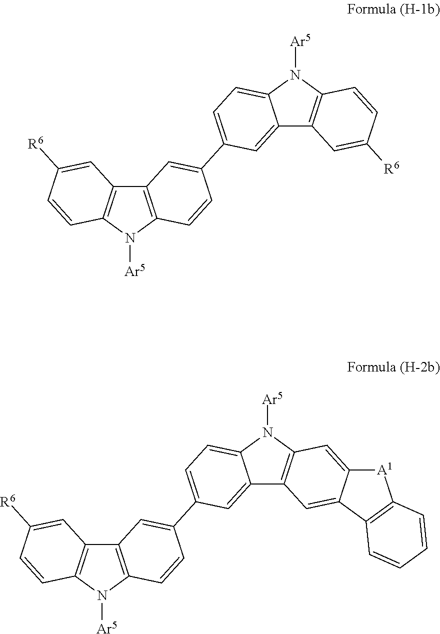

where R⁶, Ar⁵ and A¹ have the definitions given above, especially for formula (H-1) or (H-2). In a preferred embodiment of the invention, A¹ in formula (H-2b) is C(R⁷)₂.

Examples of suitable compounds of formula (H-1), (H-2), (H-3), (H-4) or (H-5) are compounds depicted in publication WO2021/043755 on pages 69 to 73 as examples of compounds of formula (6), (7), (8), (9) or (10). These compounds are incorporated into the present application by reference for purposes of disclosure.

The combination of at least one compound comprising at least one structure having at least three mutually fused structural elements of the formulae (A), (B) and (C), or the preferred embodiments thereof that are set out above, with a compound of one of the formulae (H-1), (H-2), (H-3), (H-4) and (H-5) can achieve surprising advantages. The present invention therefore further provides a composition containing at least one compound comprising at least one structure having at least three mutually fused structural elements of the formulae (A′), (B′) and (C′) or the preferred embodiments thereof that are set out above, especially compounds comprising at least one structure having at least three mutually fused structural elements of the formulae (A), (B) and (C), and at least one further matrix material, wherein the further matrix material is selected from compounds of one of the formulae (H-1), (H-2), (H-3), (H-4) and (H-5).

In a preferred configuration, it may be the case that the inventive compound comprising at least one structure having at least three mutually fused structural elements of the formulae (A′), (B′) and (C′) is used as matrix material for phosphorescent emitters in combination with a further matrix material selected from compounds of one of the formulae (H-1), (H-2), (H-3), (H-4) and (H-5). Accordingly, preference is given to electronic devices in which the compound comprising at least one structure having at least three mutually fused structural elements of the formulae (A′), (B′) and (C′) is used as matrix material for phosphorescent emitters in combination with a further matrix material selected from compounds of one of the formulae (H-1), (H-2), (H-3), (H-4) and (H-5).

It may preferably be the case that the composition consists of at least one compound comprising at least one structure having at least three mutually fused structural elements of the formulae (A′), (B′) and (C′) or the preferred embodiments thereof that are set out above, especially compounds comprising at least one structure having at least three mutually fused structural elements of the formulae (A), (B) and (C), and at least one compound of one of the formulae (H-1), (H-2), (H-3), (H-4) and (H-5). These compositions are especially suitable as what are called pre-mixtures, which can be evaporated together.

In this context, the compounds of one of the formulae (H-1), (H-2), (H-3), (H-4) and (H-5) may each be used individually or as a mixture of two, three or more compounds of the respective structures.

In addition, the compounds of the formulae (H-1), (H-2), (H-3), (H-4) and (H-5) may be used individually or as a mixture of two, three or more compounds of different structures.

The compound containing at least one compound comprising at least one structure having at least three mutually fused structural elements of the formulae (A′), (B′) and (C′) or the preferred embodiments thereof that are set out above, especially compounds comprising at least one structure having at least three mutually fused structural elements of the formulae (A), (B) and (C), preferably has a proportion by mass in the composition in the range from 10% by weight to 95% by weight, more preferably in the range from 15% by weight to 90% by weight, and very preferably in the range from 40% by weight to 70% by weight, based on the total mass of the composition. Compounds comprising at least one structure having at least three mutually fused structural elements of the formulae (A′), (B′) and (C′) may be used individually or as a mixture of two, three or more compounds.

It may further be the case that the compounds of one of the formulae (H-1), (H-2), (H-3), (H-4) and (H-5) have a proportion by mass in the composition in the range from 5% by weight to 90% by weight, preferably in the range from 10% by weight to 85% by weight, more preferably in the range from 20% by weight to 85% by weight, even more preferably in the range from 30% by weight to 80% by weight, very particularly preferably in the range from 20% by weight to 60% by weight and most preferably in the range from 30% by weight to 50% by weight, based on the overall composition.

It may additionally be the case that the composition consists exclusively of compounds comprising at least one structure having at least three mutually fused structural elements of the formulae (A′), (B′) and (C′), or the preferred embodiments thereof that are set out above, and one of the further matrix materials mentioned, preferably compounds of at least one of the formulae (H-1), (H-2), (H-3), (H-4) and (H-5).

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

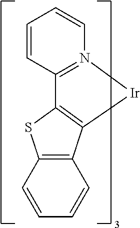

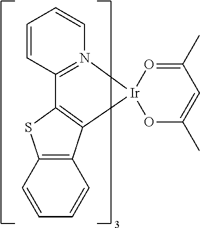

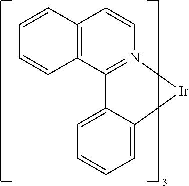

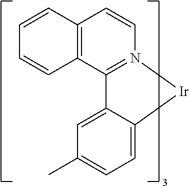

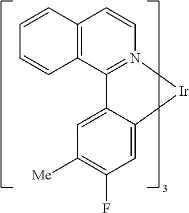

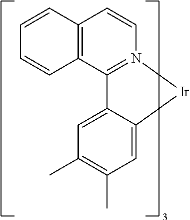

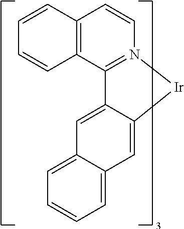

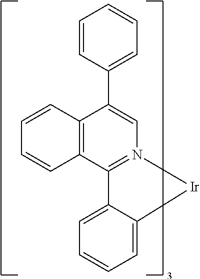

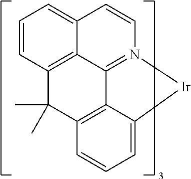

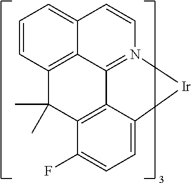

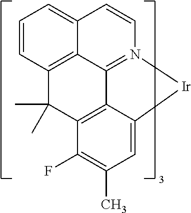

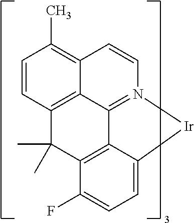

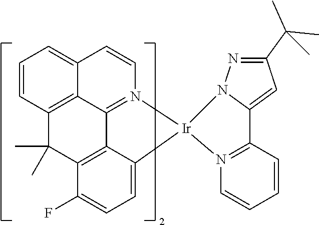

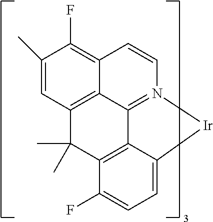

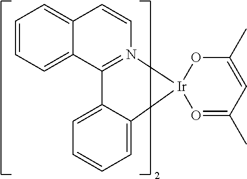

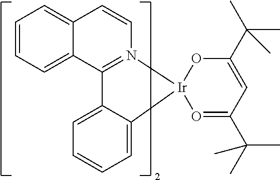

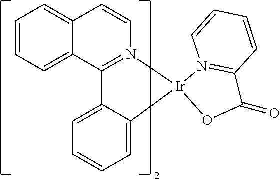

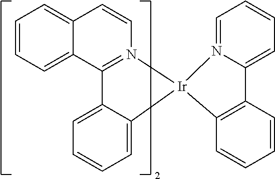

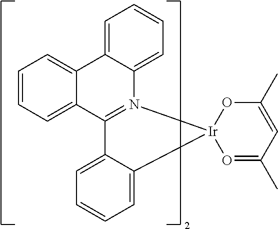

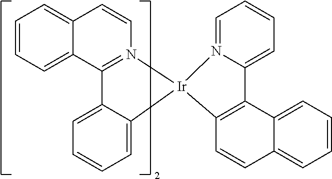

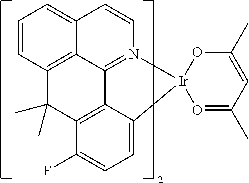

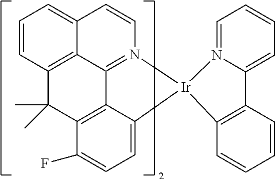

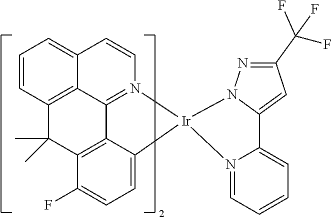

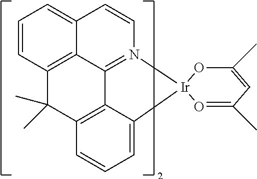

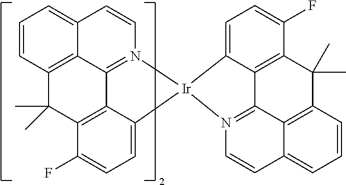

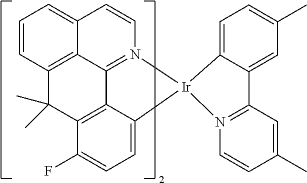

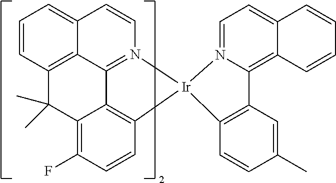

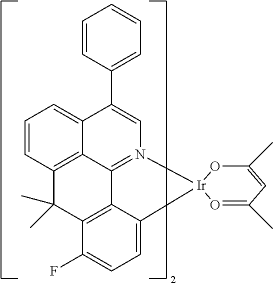

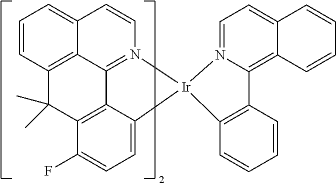

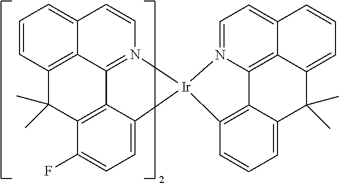

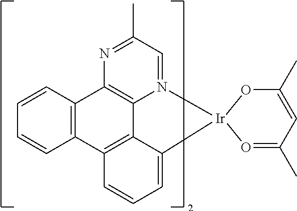

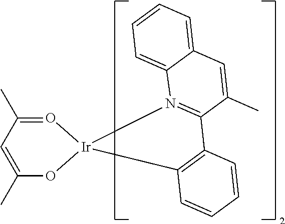

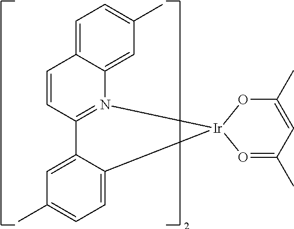

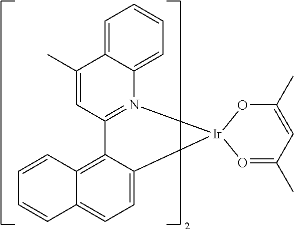

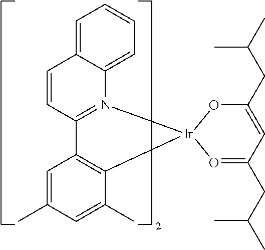

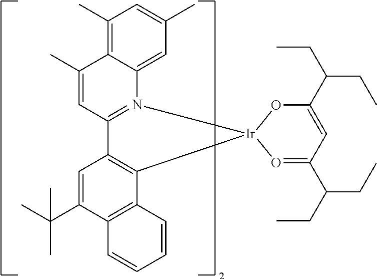

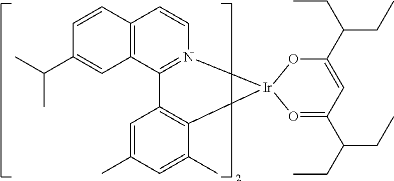

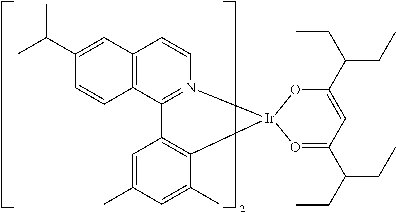

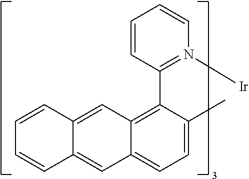

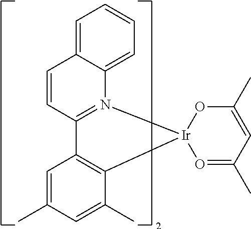

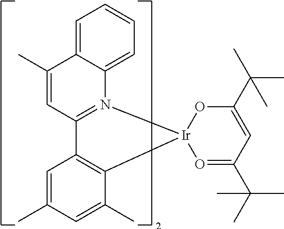

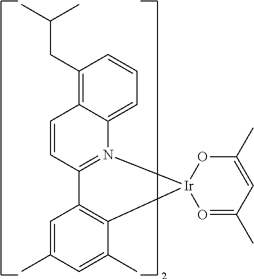

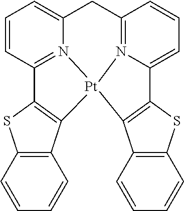

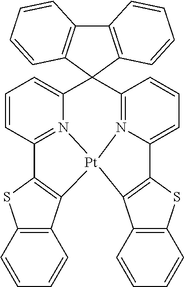

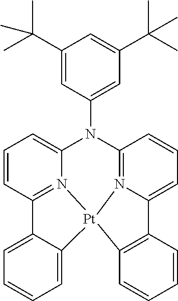

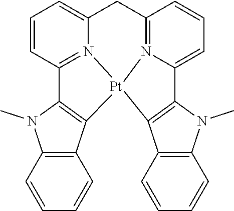

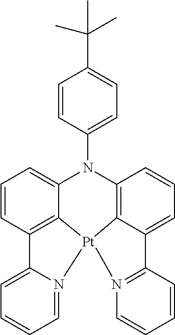

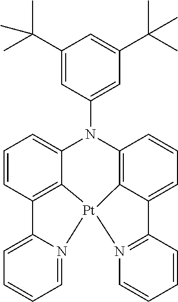

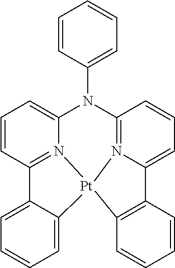

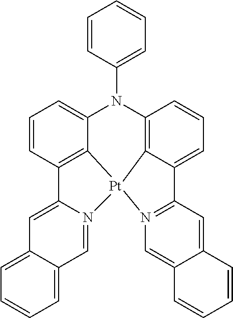

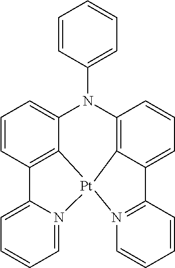

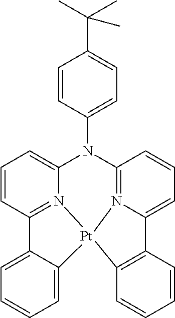

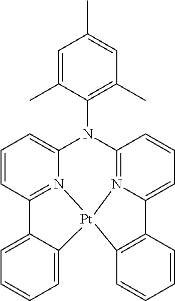

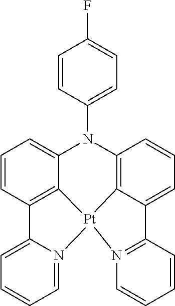

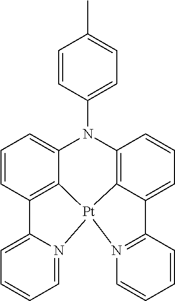

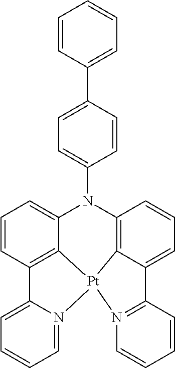

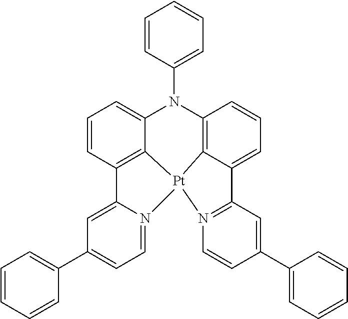

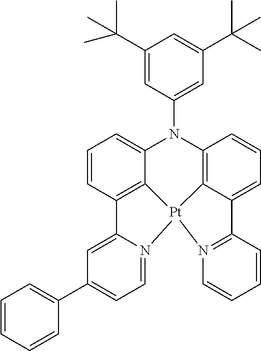

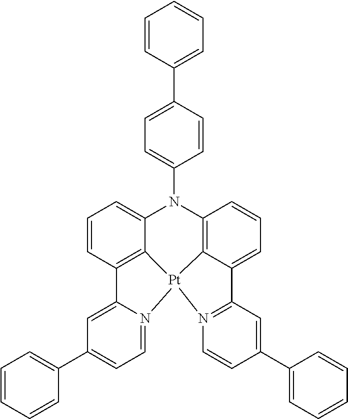

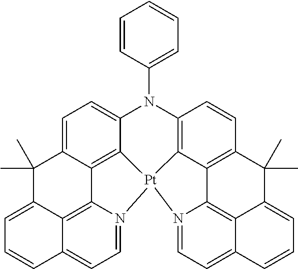

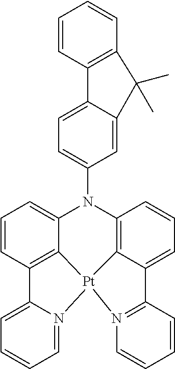

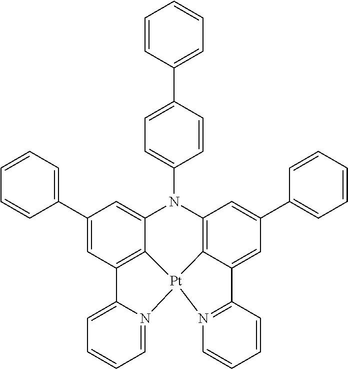

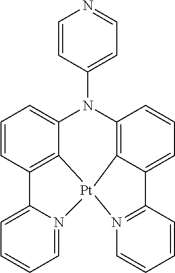

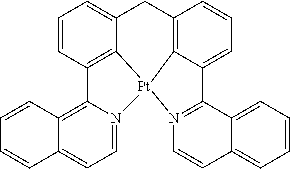

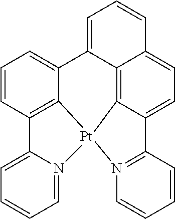

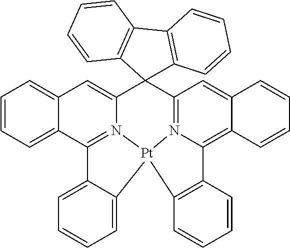

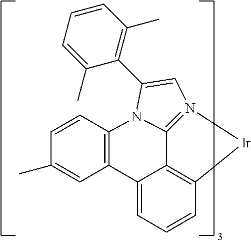

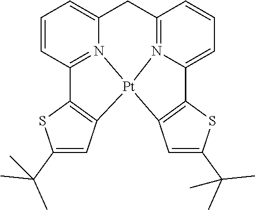

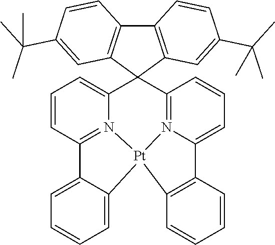

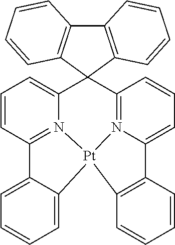

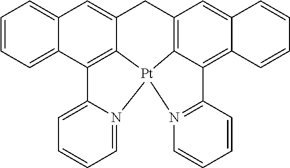

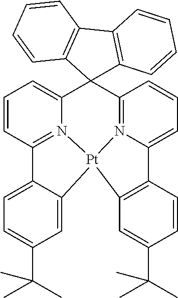

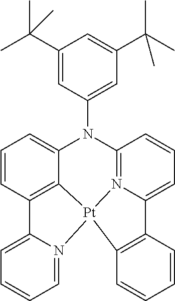

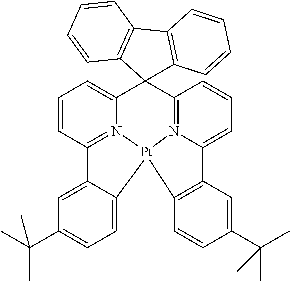

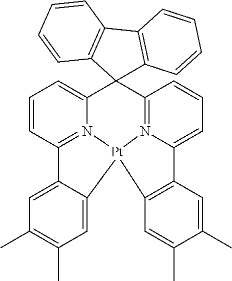

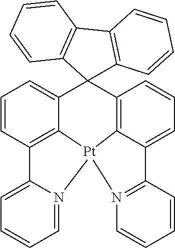

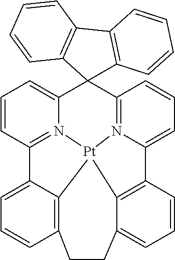

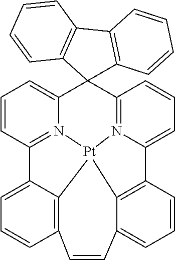

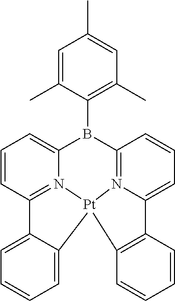

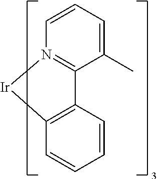

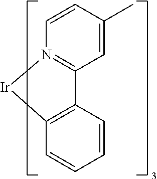

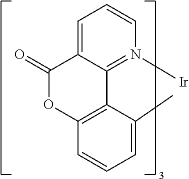

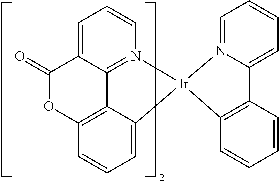

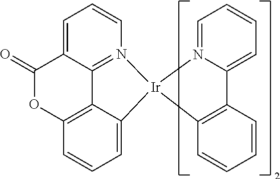

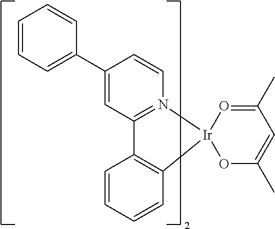

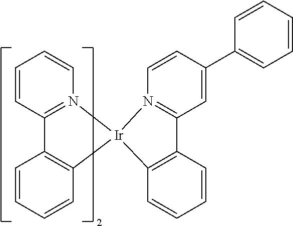

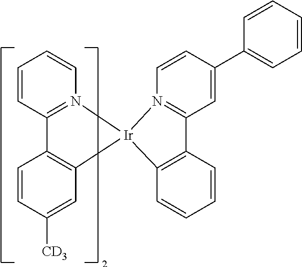

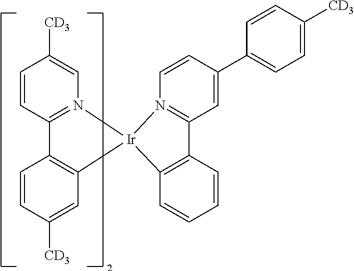

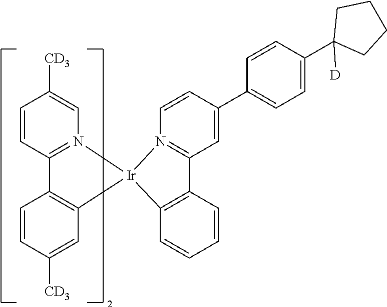

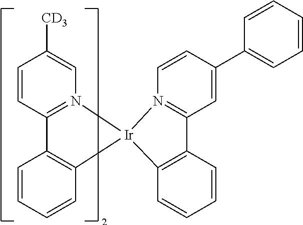

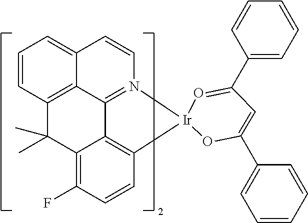

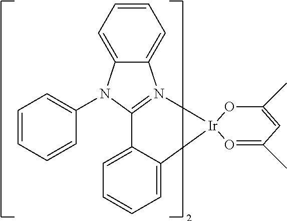

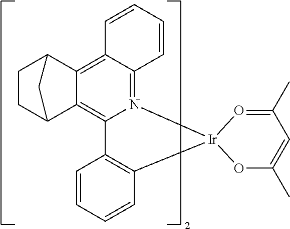

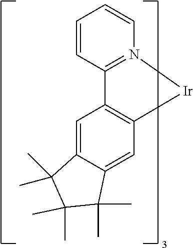

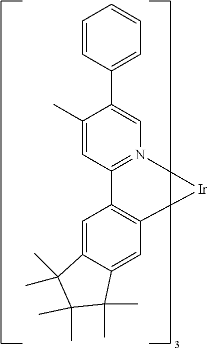

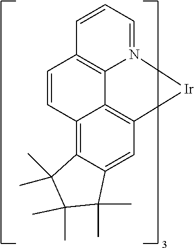

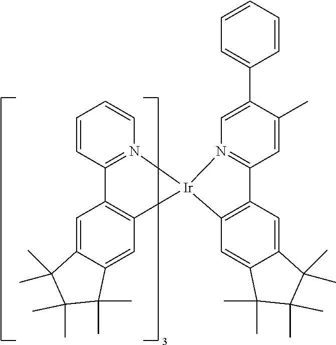

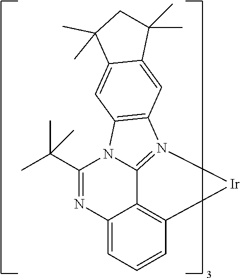

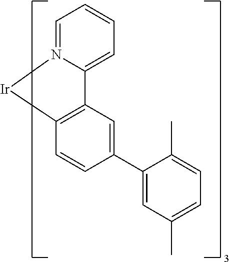

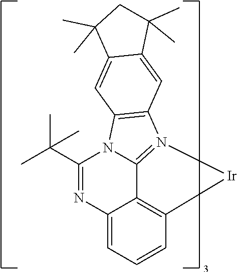

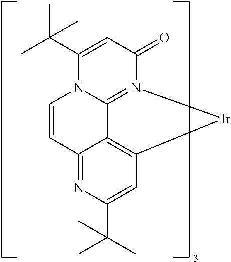

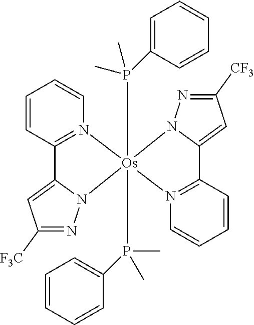

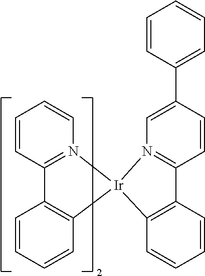

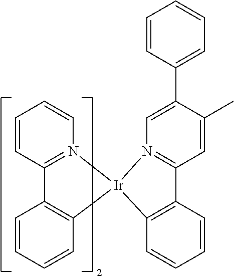

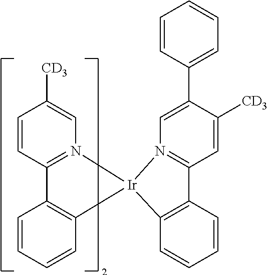

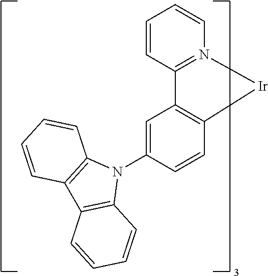

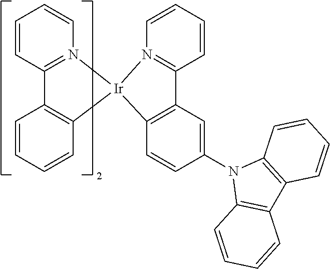

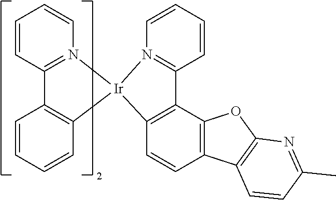

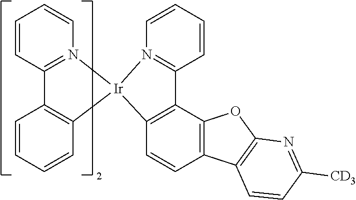

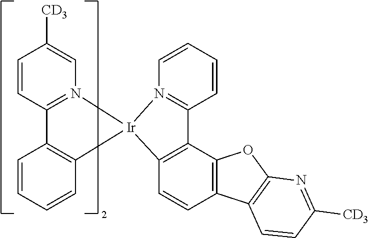

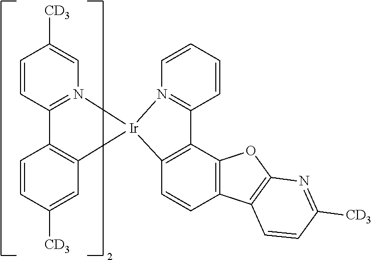

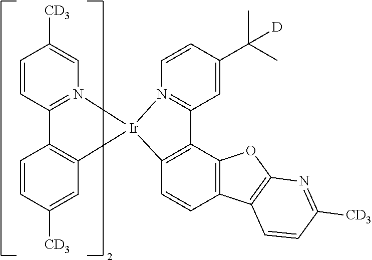

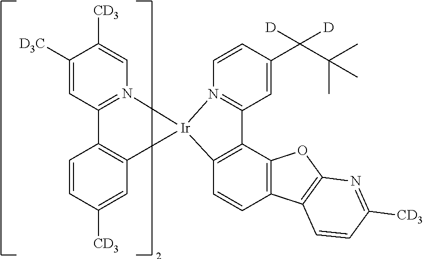

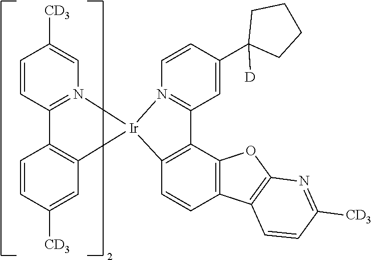

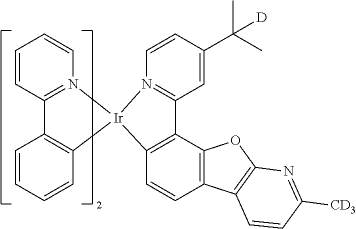

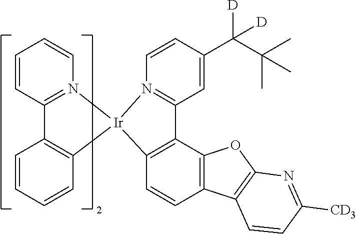

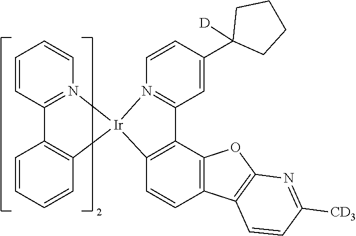

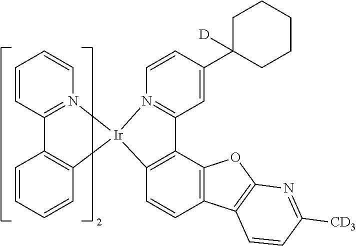

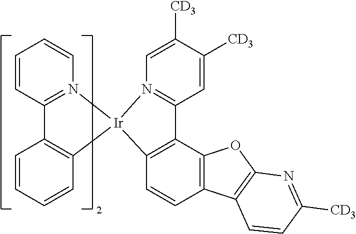

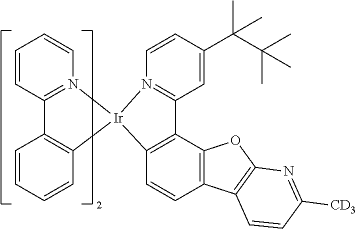

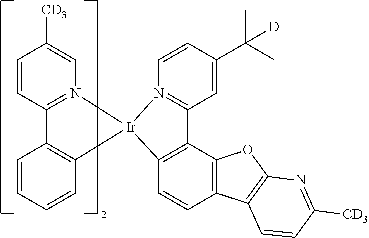

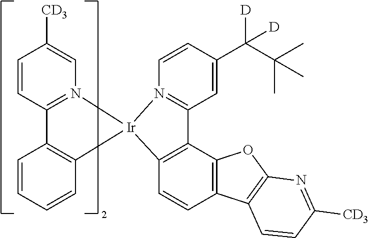

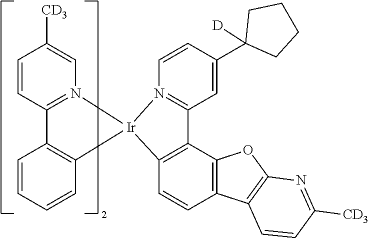

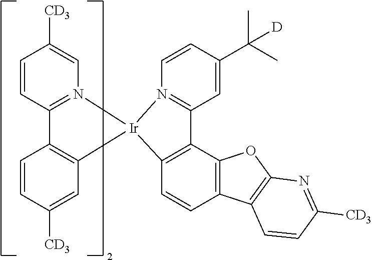

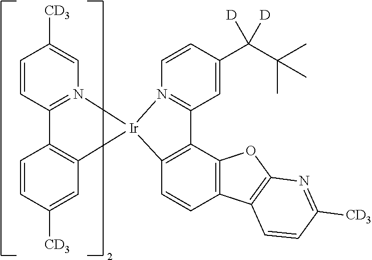

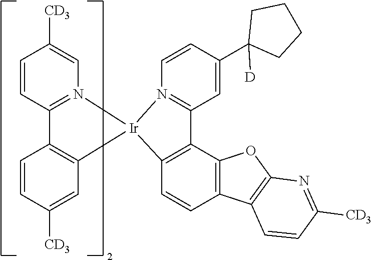

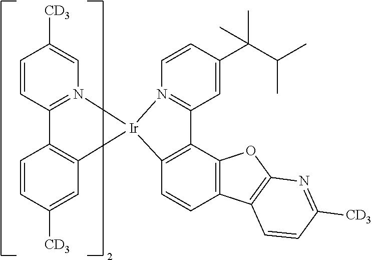

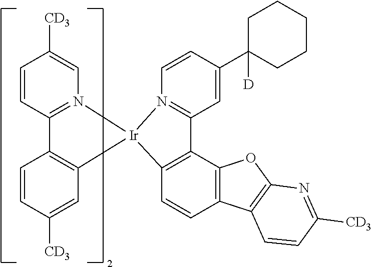

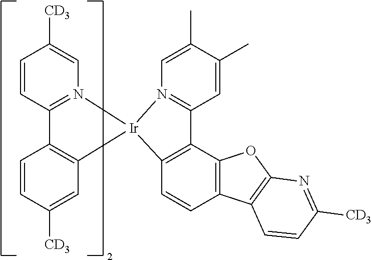

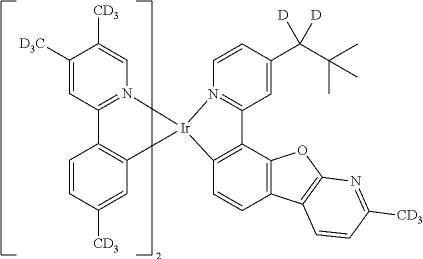

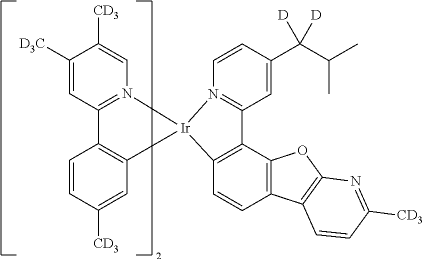

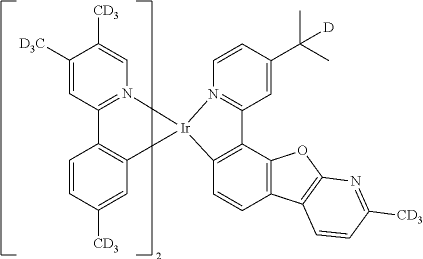

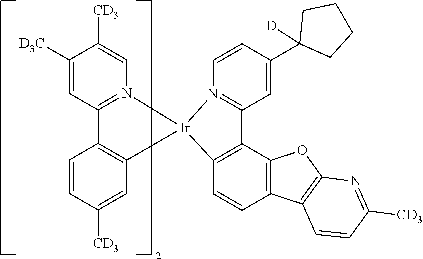

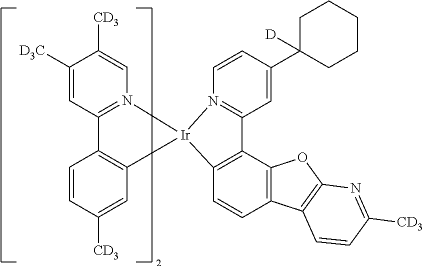

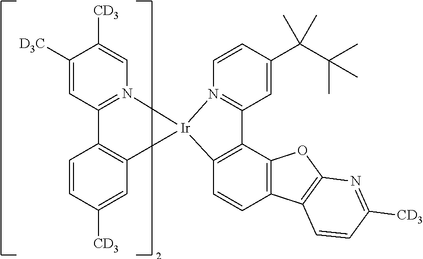

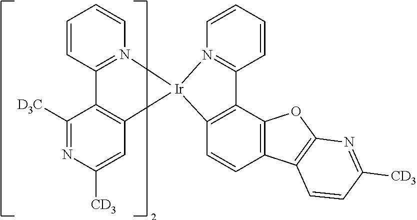

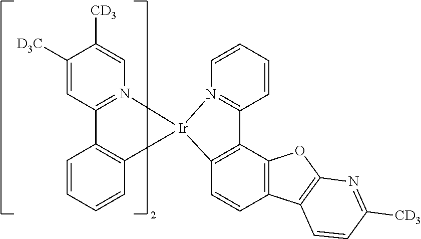

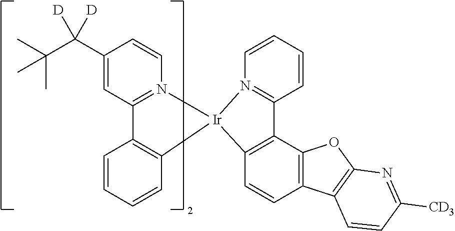

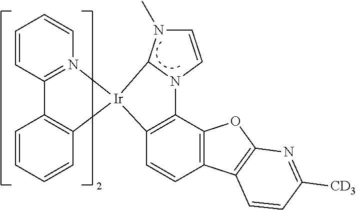

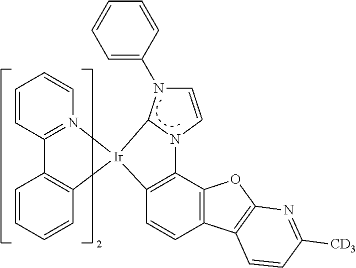

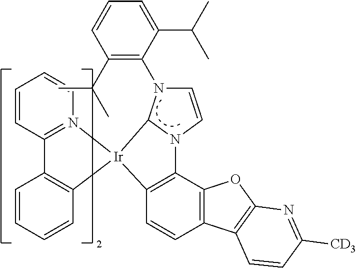

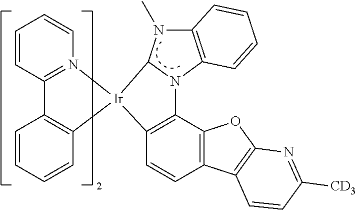

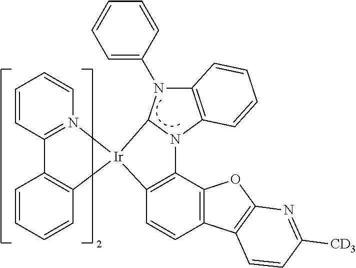

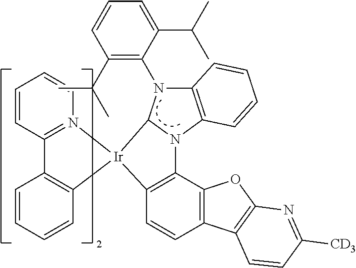

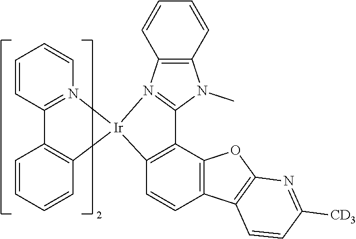

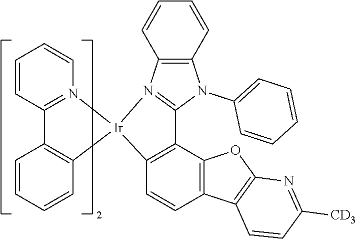

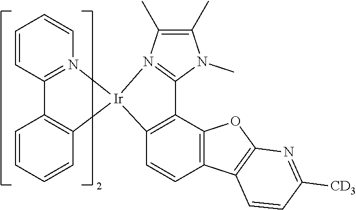

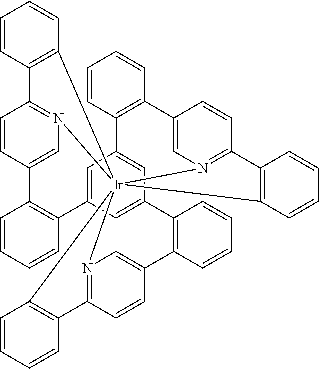

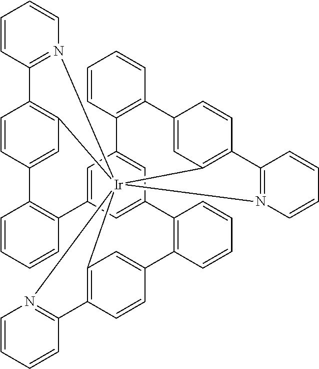

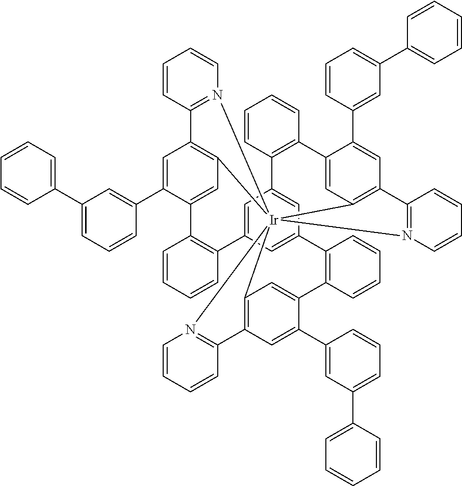

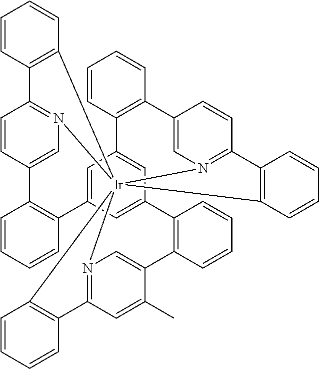

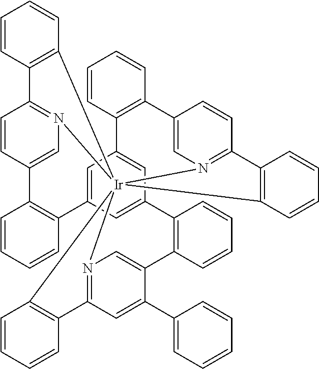

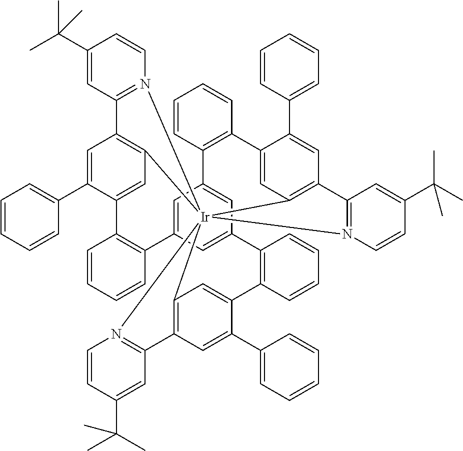

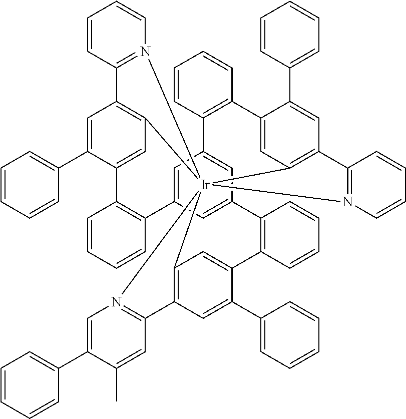

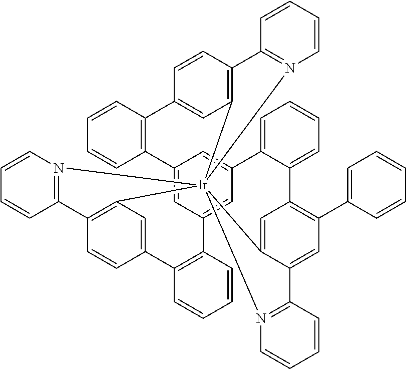

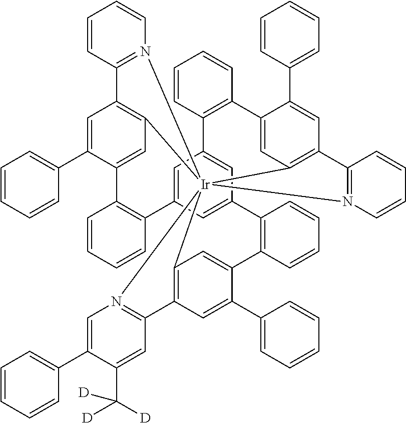

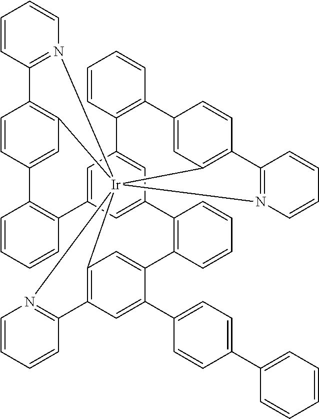

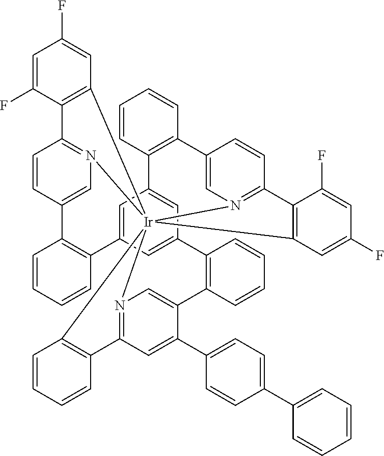

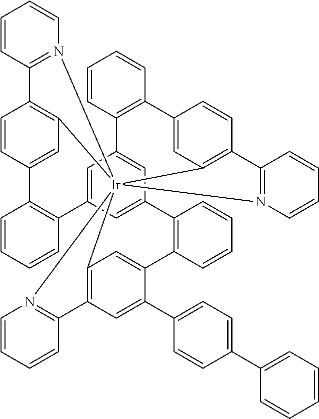

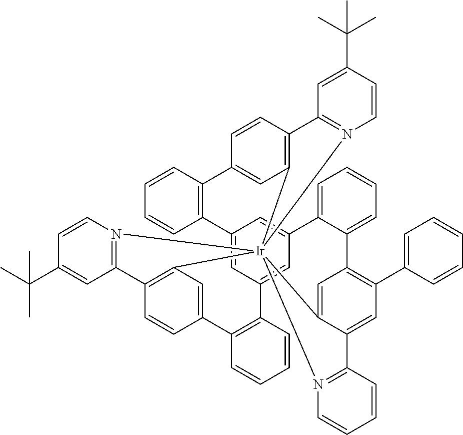

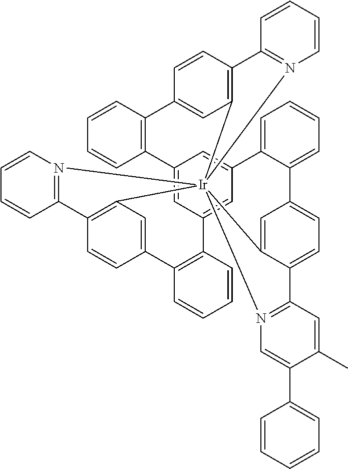

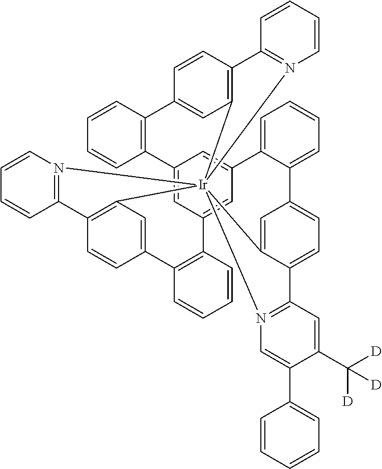

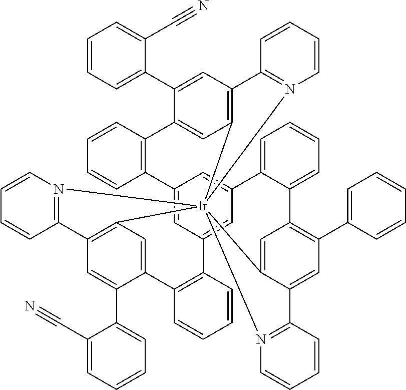

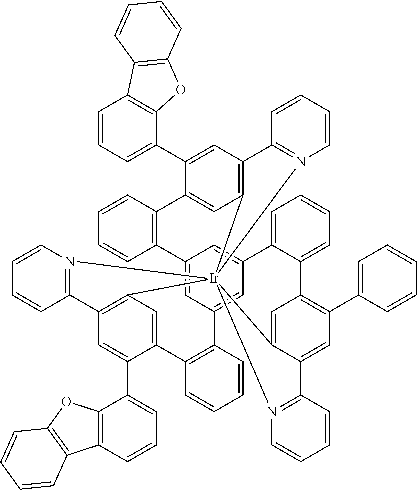

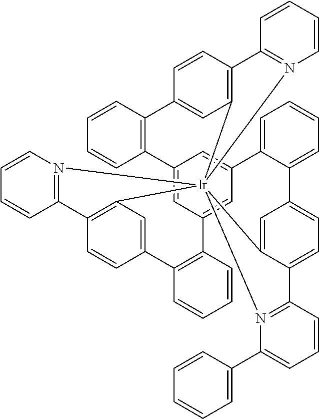

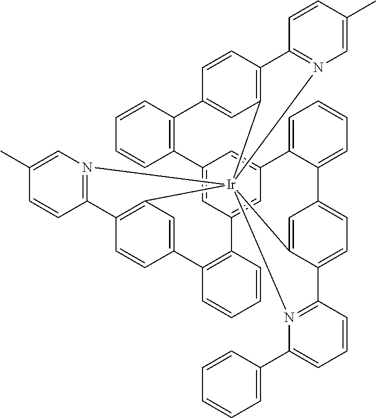

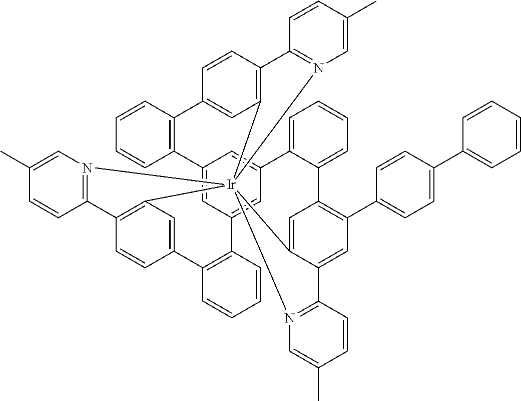

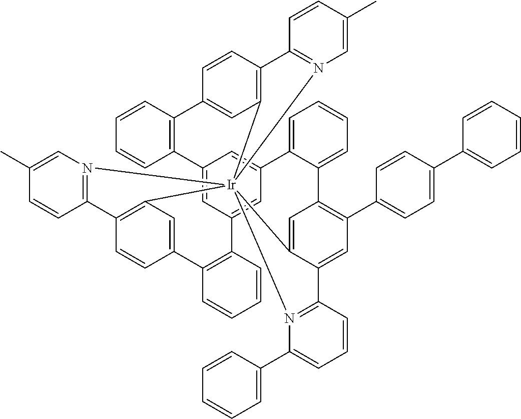

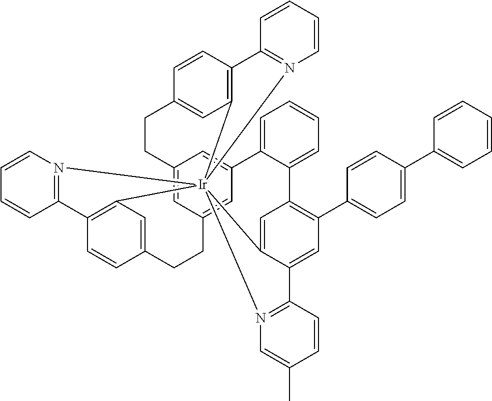

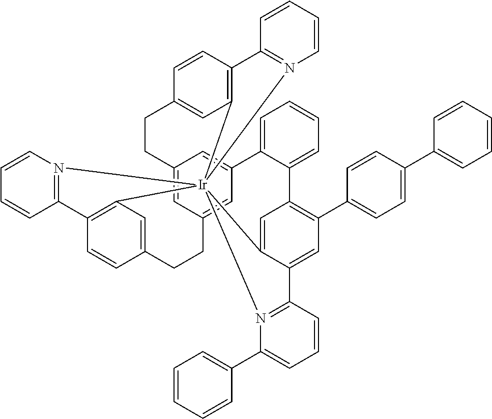

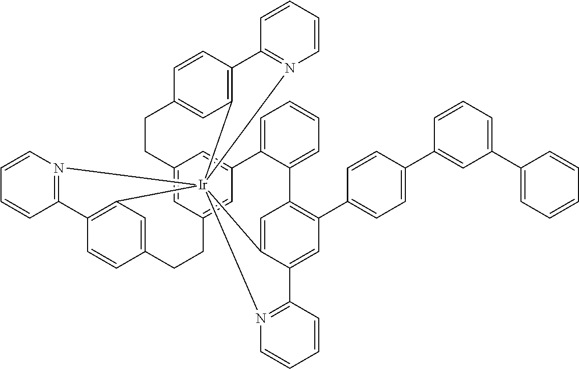

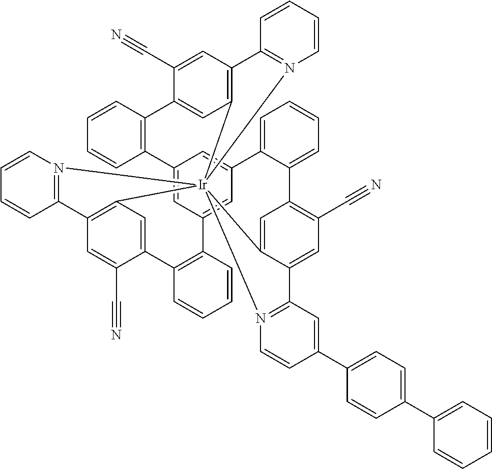

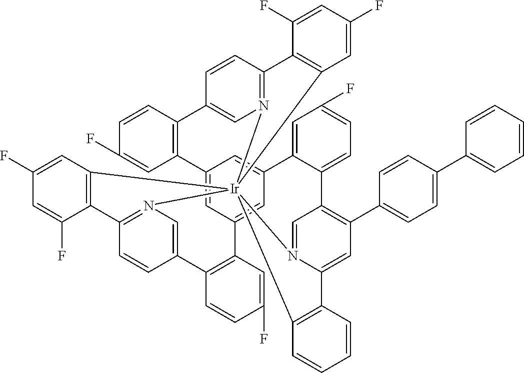

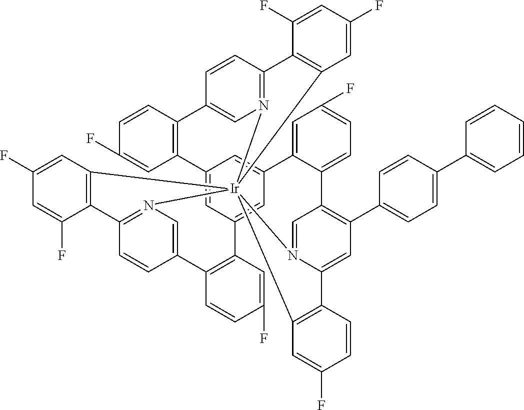

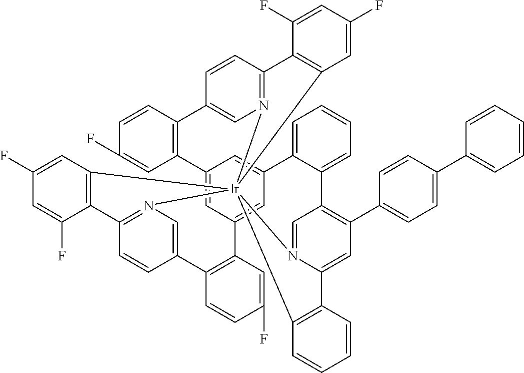

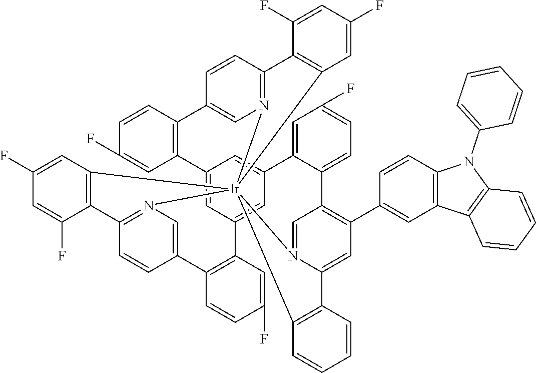

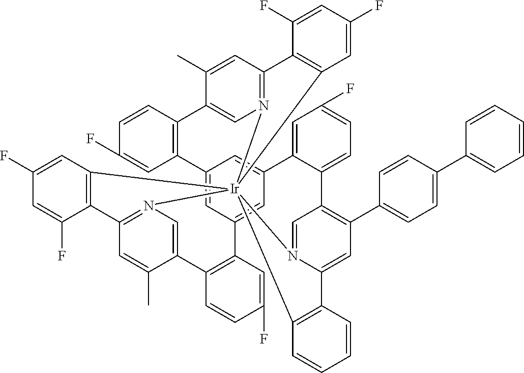

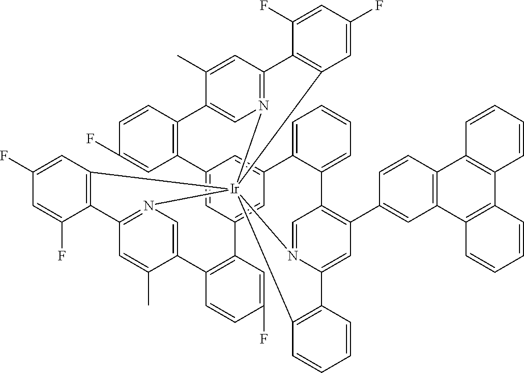

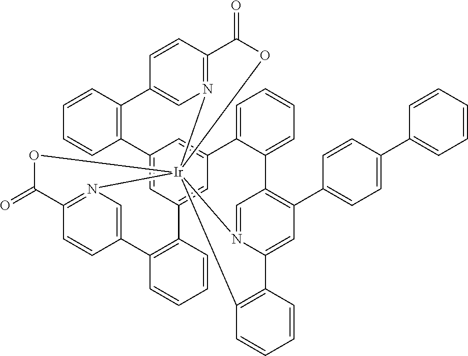

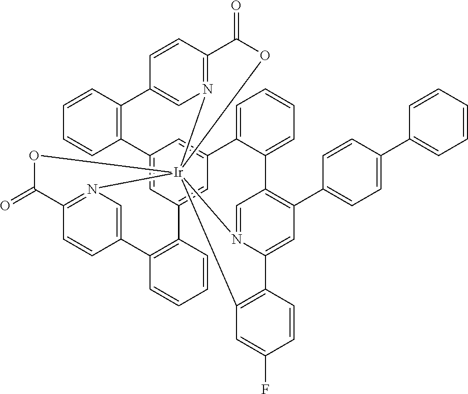

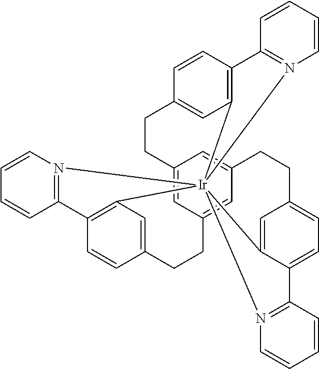

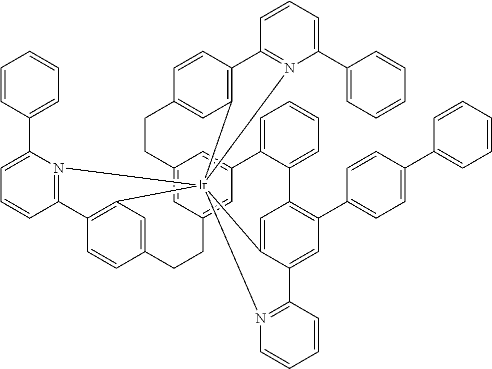

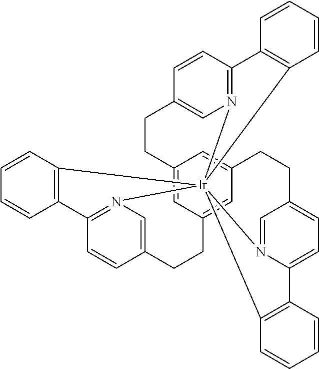

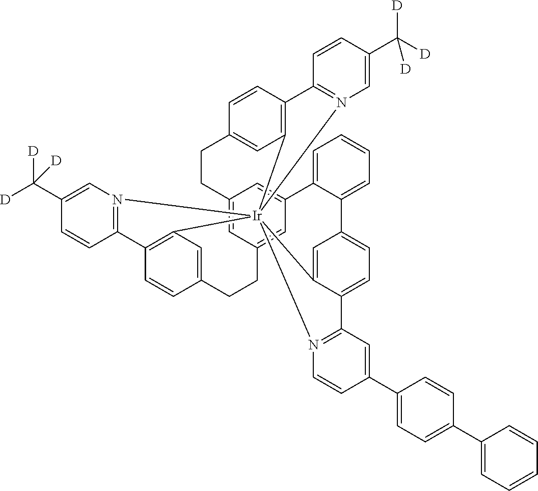

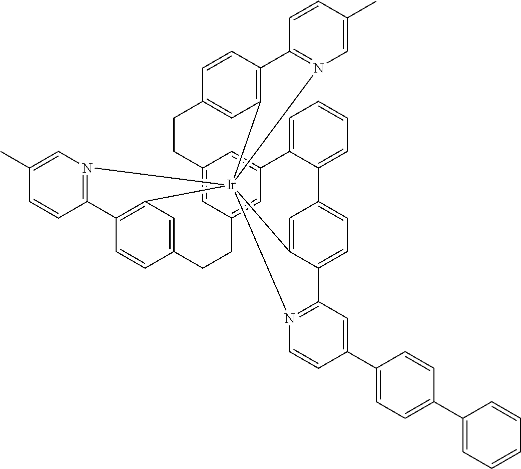

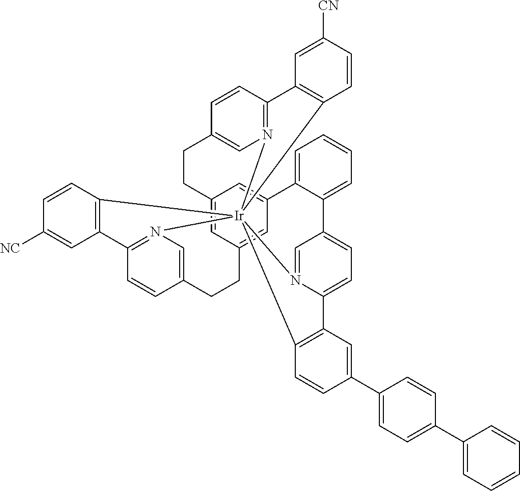

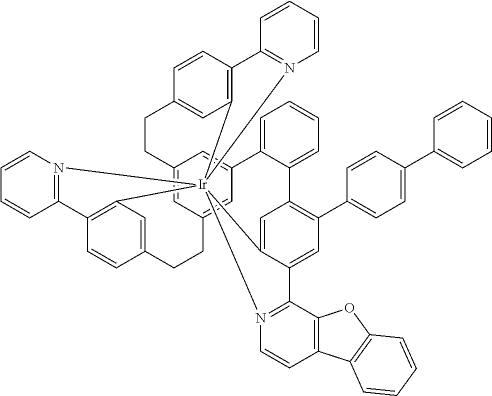

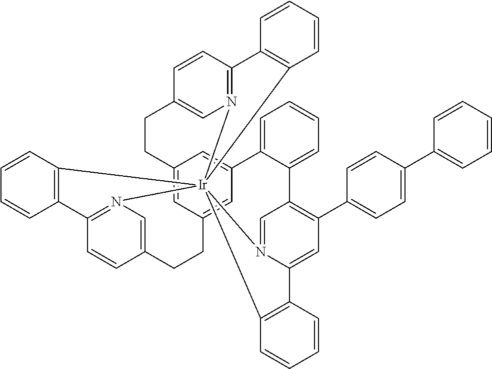

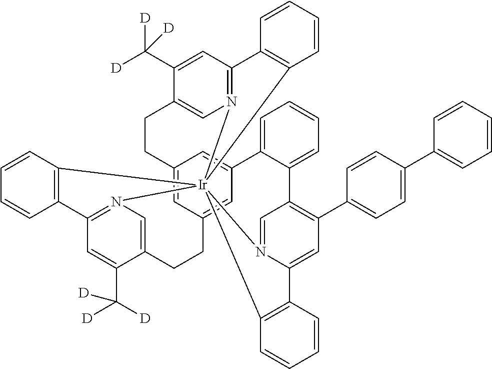

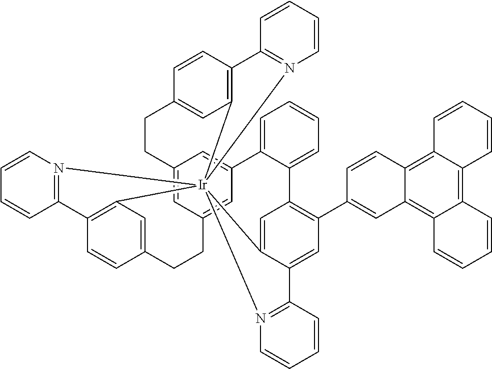

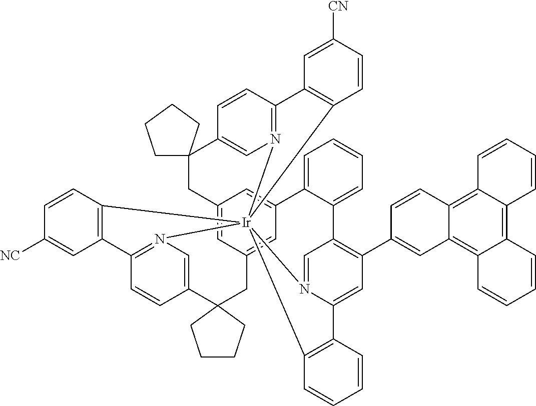

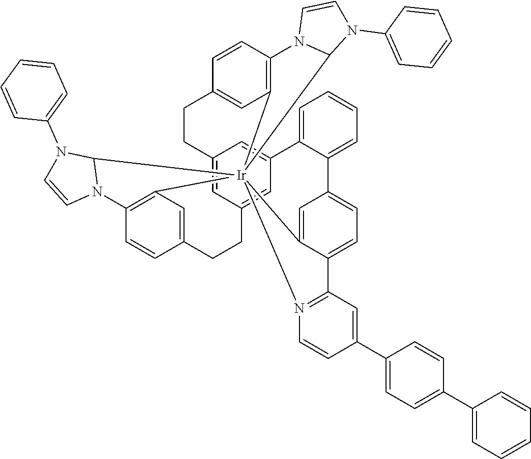

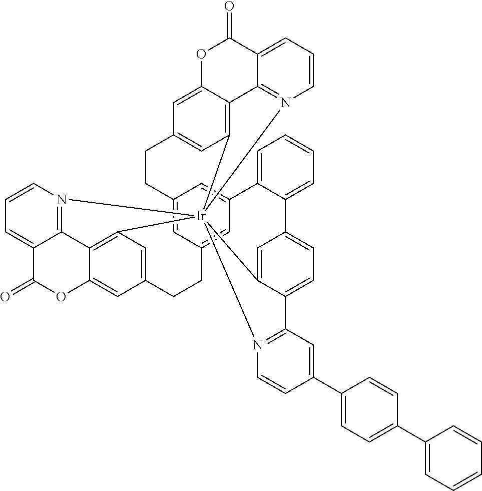

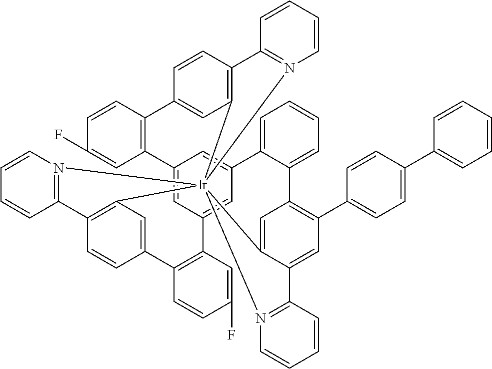

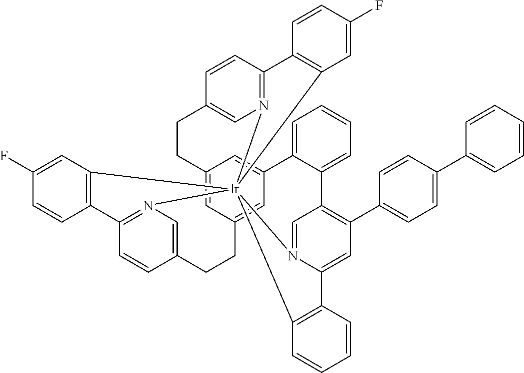

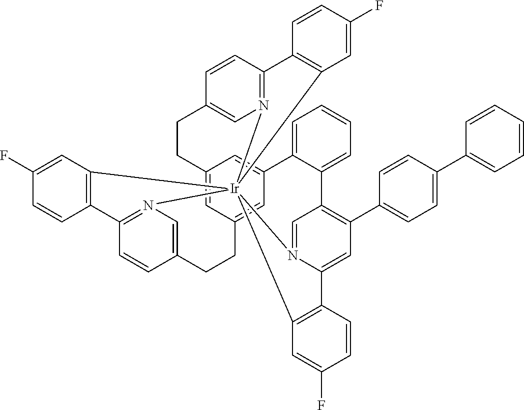

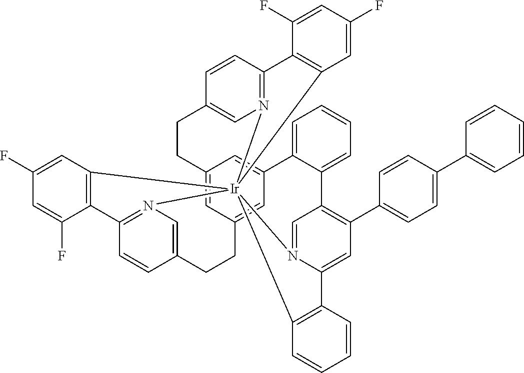

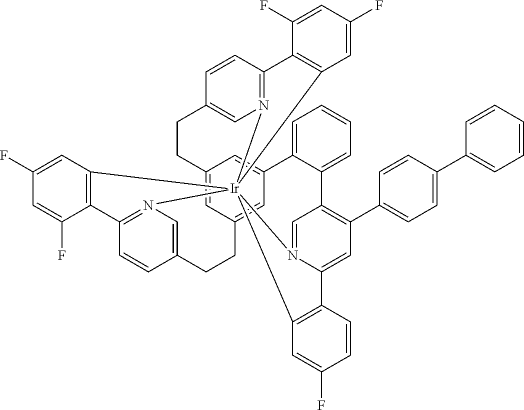

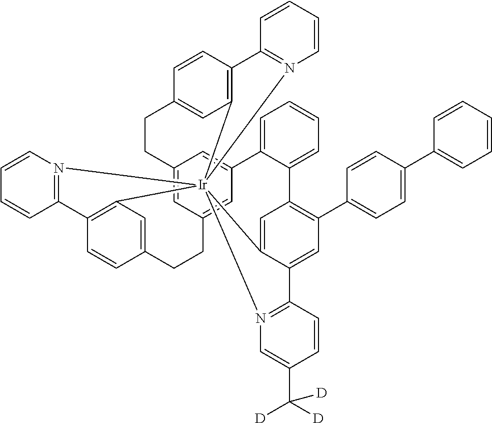

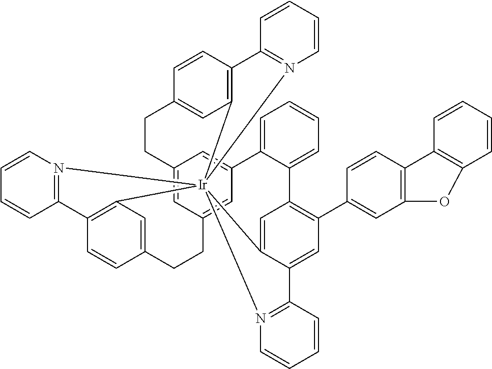

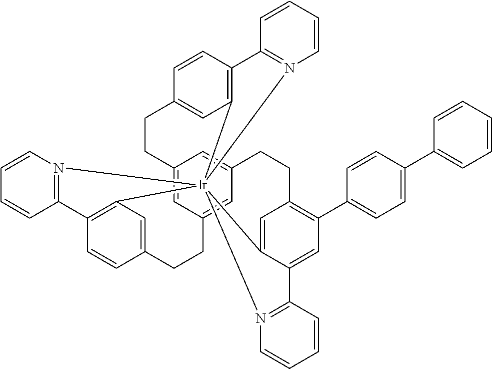

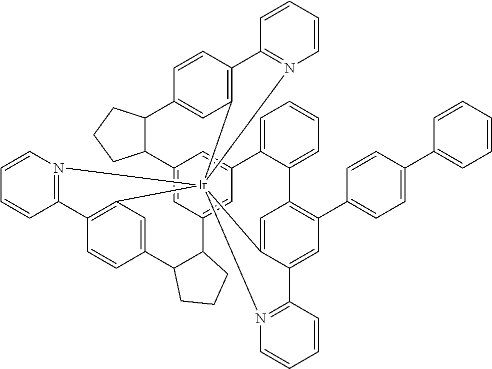

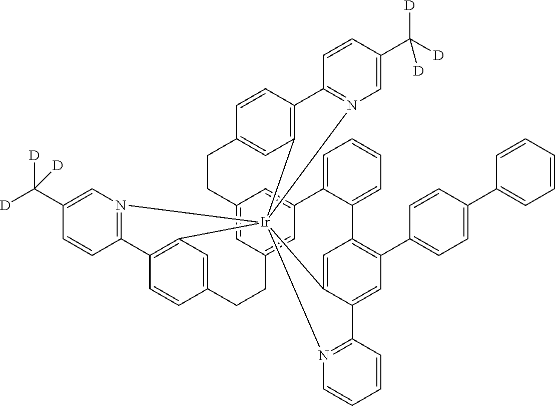

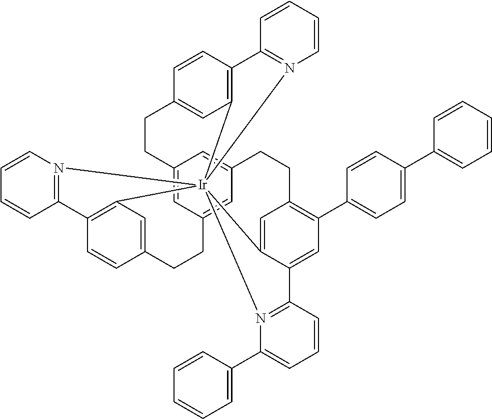

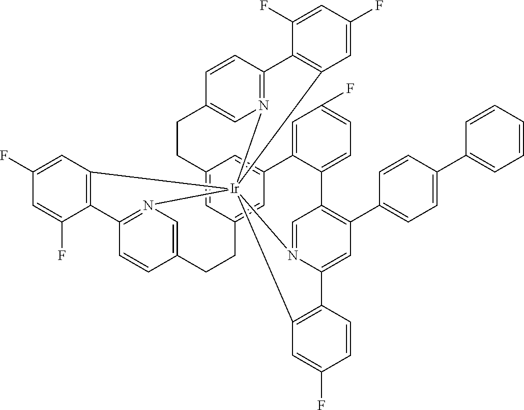

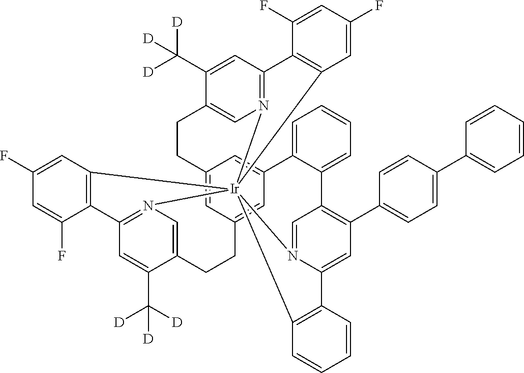

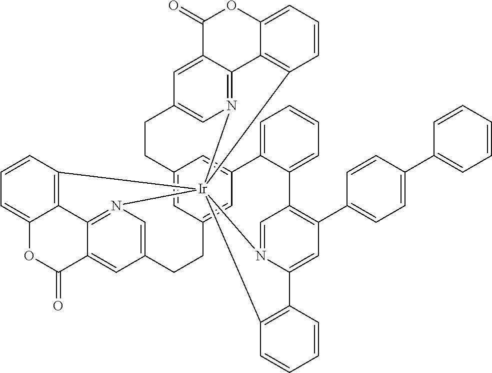

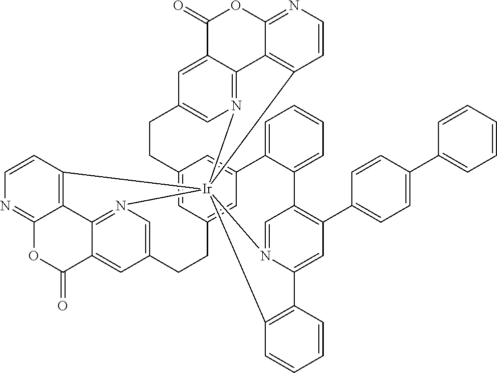

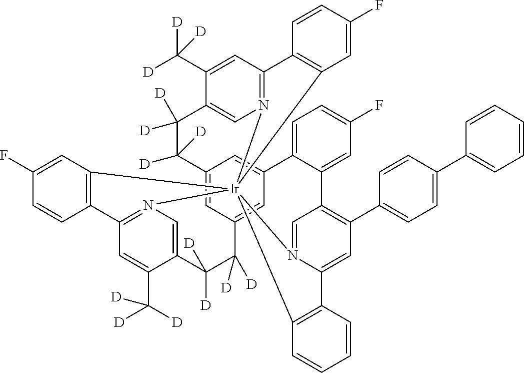

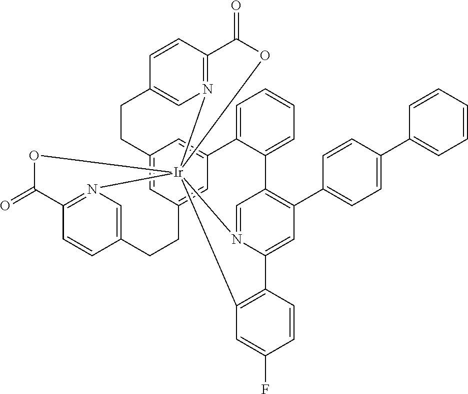

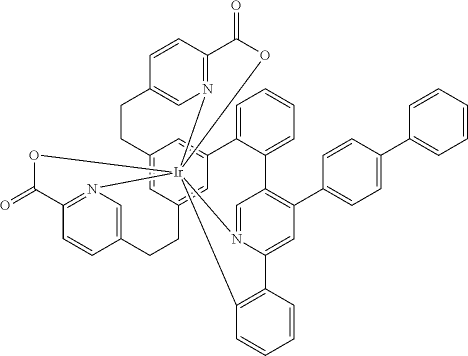

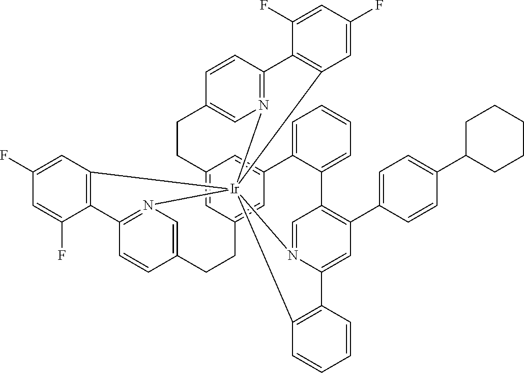

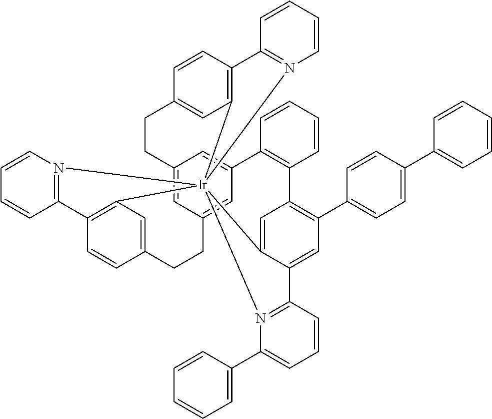

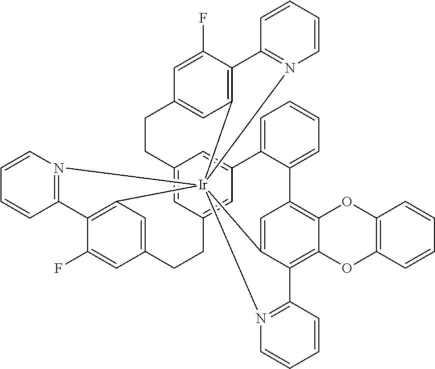

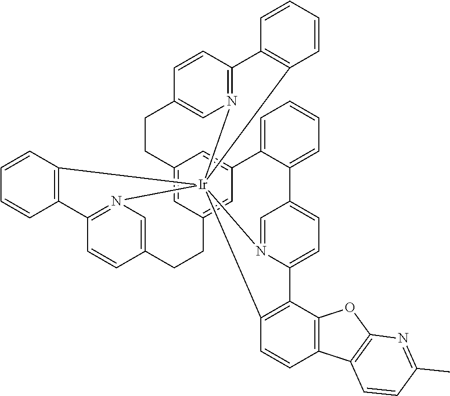

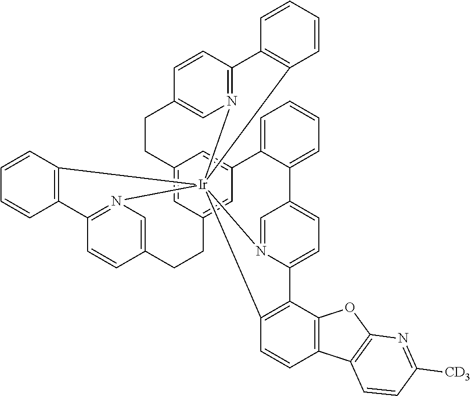

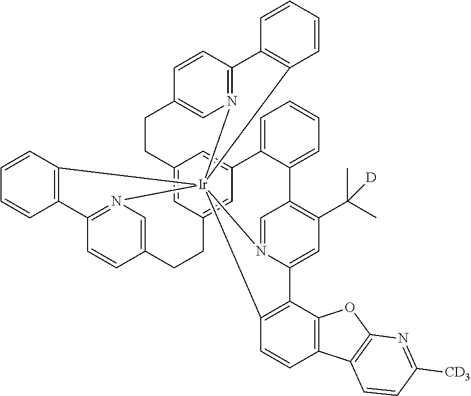

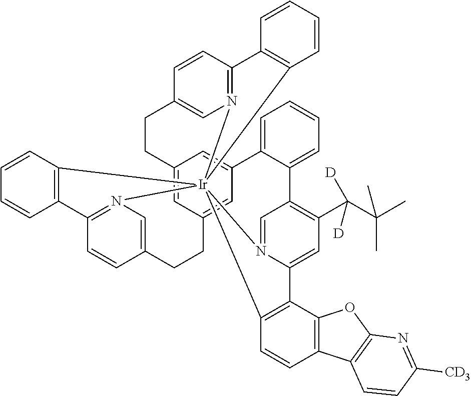

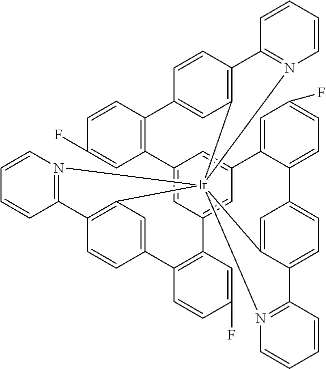

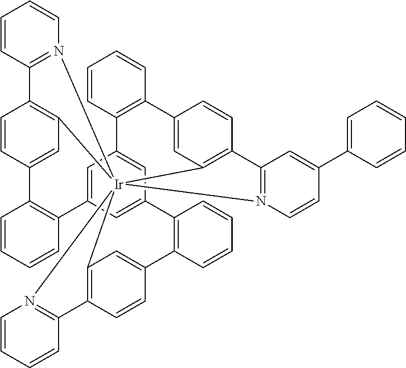

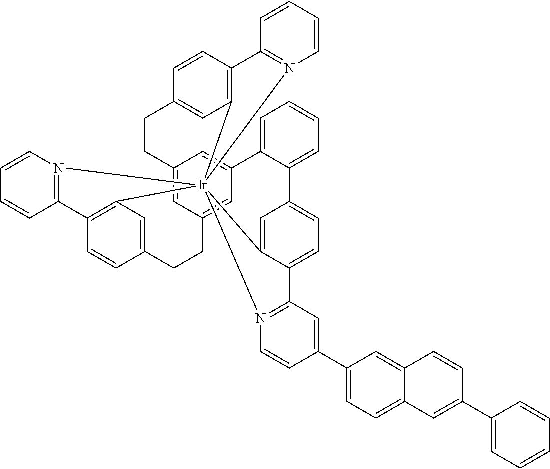

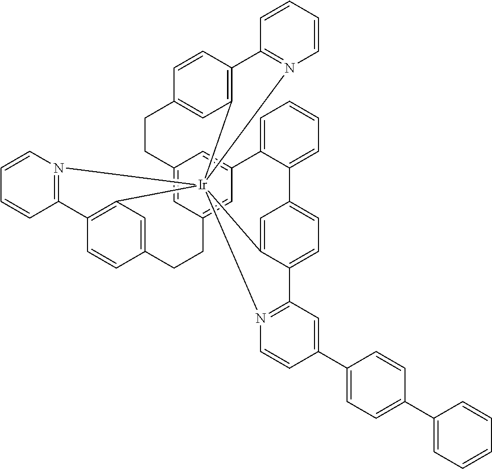

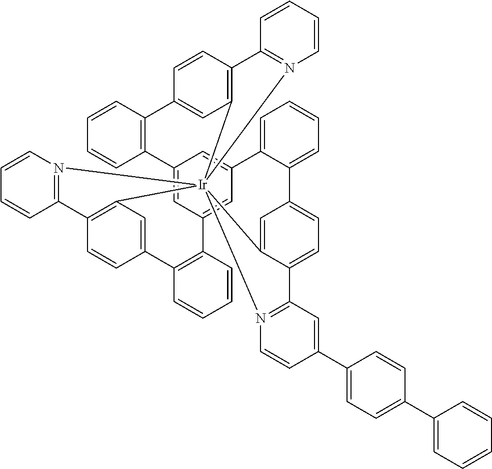

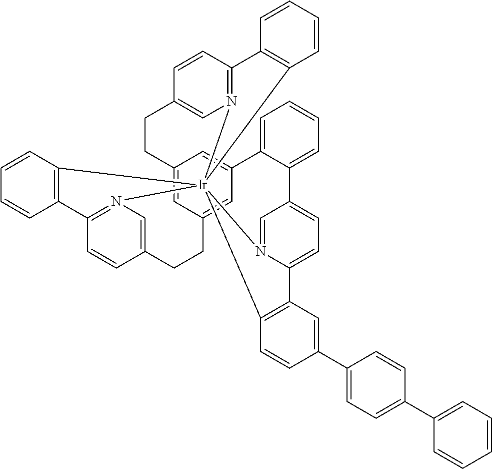

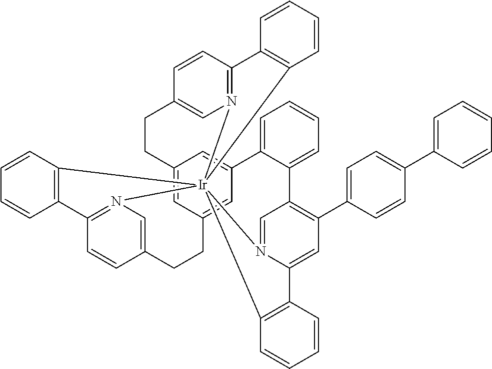

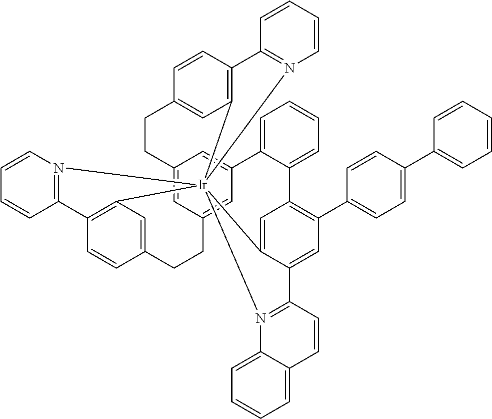

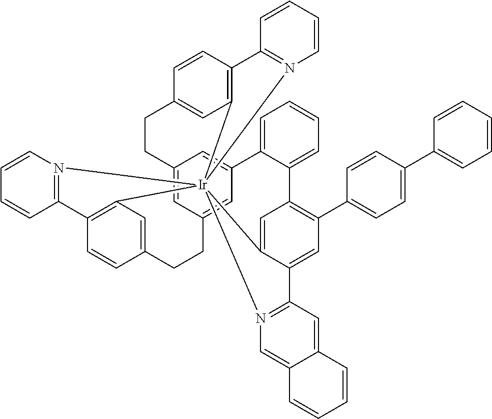

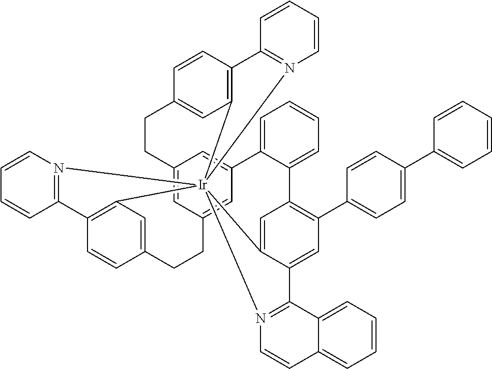

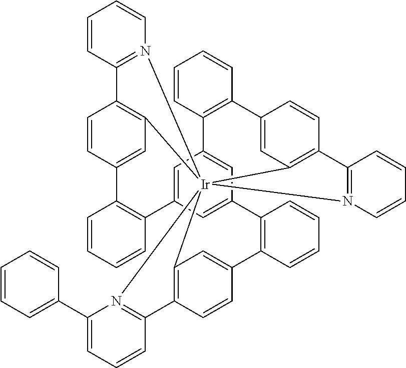

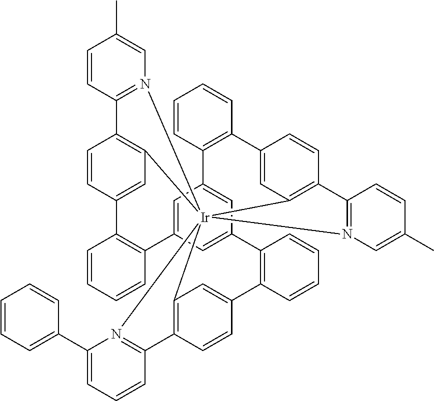

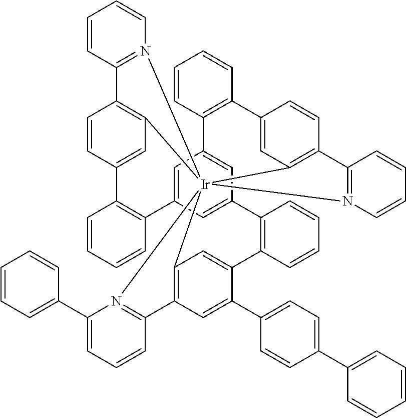

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds comprising at least one structure having at least three mutually fused structural elements of the formulae (A′), (B′) and (C′) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for applying a compound comprising at least one structure having at least three mutually fused structural elements of the formulae (A), (B) and (C), or the preferred embodiments thereof that are set out above, are novel. The present invention therefore further provides a formulation comprising at least one solvent and a compound comprising at least one structure having at least three mutually fused structural elements of the formulae (A), (B) and (C) or the preferred embodiments thereof that are set out above. The present invention further provides a formulation comprising at least one solvent and a compound comprising at least one structure having at least three mutually fused structural elements of the formulae (A′), (B′) and (C′), or the preferred embodiments thereof that are set out above, and a compound of at least one of the formulae (H-1), (H-2), (H-3), (H-4) and (H-5).

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices, containing compounds comprising at least one structure having at least three mutually fused structural elements of the formulae (A′), (B′) and (C′) or the preferred embodiments recited above and hereinafter, especially as matrix material or as electron-conducting materials, have a very good lifetime. In this context, these compounds especially bring about low roll-off, i.e. a small drop in power efficiency of the device at high luminances.
2. Electronic devices, especially organic electroluminescent devices, containing compounds comprising at least one structure having at least three mutually fused structural elements of the formulae (A′), (B′) and (C′) or the preferred embodiments recited above and hereinafter as electron-conducting materials and/or matrix materials have excellent efficiency. In this context, inventive compounds comprising at least one structure having at least three mutually fused structural elements of the formulae (A), (B) and (C) or the preferred embodiments recited above and hereinafter bring about a low operating voltage when used in electronic devices.
3. The inventive compounds comprising at least one structure having at least three mutually fused structural elements of the formulae (A), (B) and (C) or the preferred embodiments recited above and hereinafter exhibit very high stability and lifetime.
4. With compounds comprising at least one structure having at least three mutually fused structural elements of the formulae (A), (B) and (C) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. Compounds comprising at least one structure having at least three mutually fused structural elements of the formulae (A), (B) and (C) or the preferred embodiments recited above and hereinafter have excellent glass film formation.
6. Compounds comprising at least one structure having at least three mutually fused structural elements of the formulae (A), (B) and (C) or the preferred embodiments recited above and hereinafter form very good films from solutions.
7. Electronic devices, especially organic electroluminescent devices comprising compounds comprising at least one structure having at least three mutually fused structural elements of the formulae (A′), (B′) and (C′) or the preferred embodiments detailed above and hereinafter, in combination with host materials of one or more of the formulae (H-1) to (H-5), especially as matrix material, have an improved lifetime and higher efficiency.
8. The compounds comprising at least one structure having at least three mutually fused structural elements of the formulae (A), (B) and (C) or the preferred embodiments recited above and hereinafter have a low triplet level Ti which may, for example, be in the range of −2.22 eV to −2.9 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention.

Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### SYNTHESIS EXAMPLES

### a) 2-(2-Fluorophenyl)-3-methylisoquinolin-1-one

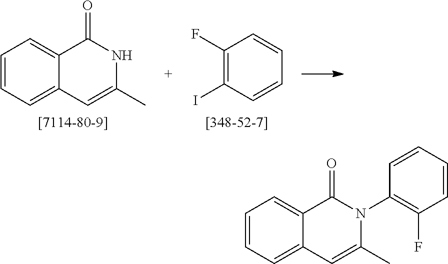 [SMILES: Cc1cc2ccccc2c(=O)[nH]1 ; Fc1ccccc1I ; Cc1cc2ccccc2c(=O)n1-c1ccccc1F]

6.3 g (40 mmol) of 3-methyl-1(2H)-isoquinolinone and 9.9 g (45 mmol) of 1-fluoro-2-iodobenzene and 44.7 g (320 mmol) of potassium carbonate, 3 g (16 mmol) of copper(I) iodide and 3.6 g (16 mmol) of 1,3-di(pyridin-2-yl)propane-1,3-dione are stirred in 100 ml of DMF at 150° C. for 30 h. The solution is diluted with water and extracted twice with ethyl acetate, and the combined organic phases are dried over Na₂SO₄ and concentrated. The residue is purified by chromatography (EtOAc/hexane: 2/3), recrystallized from toluene and finally purified under high vacuum (p=5×10⁻⁵ mbar). The purity is 99.9%. The yield is 8.1 g (30 mmol), 77% of theory.

The following compounds are prepared in an analogous manner:

| Ex. | Reactant 1 | Reactant 2 | Product | Yield |
|---|---|---|---|---|
| 1a | 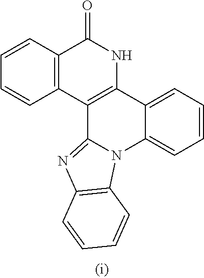 [SMILES: O=c1[nH]c2c3ccccc3n3c4ccccc4nc3c2c2ccccc12] | 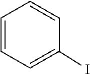 | 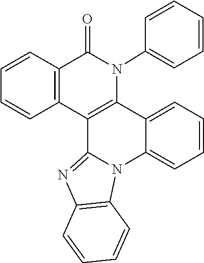 [SMILES: O=c1c2ccccc2c2c(c3ccccc3n3c4ccccc4nc23)n1-c1ccccc1] | 75% |
| 2a | 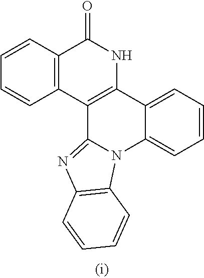 [SMILES: O=c1[nH]c2c3ccccc3n3c4ccccc4nc3c2c2ccccc12] | 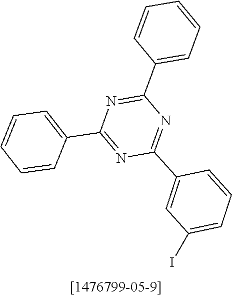 [SMILES: Ic1cccc(-c2nc(-c3ccccc3)nc(-c3ccccc3)n2)c1] | 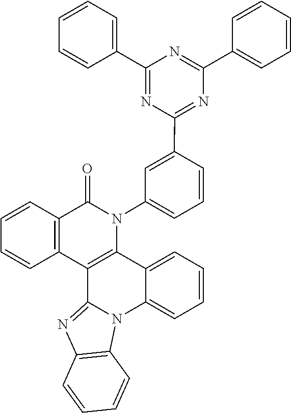 [SMILES: O=c1c2ccccc2c2c(c3ccccc3n3c4ccccc4nc23)n1-c1cccc(-c2nc(-c3ccccc3)nc(-c3ccccc3)n2)c1] | 68% |
| 3a | 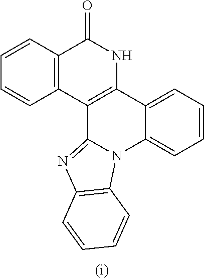 [SMILES: O=c1[nH]c2c3ccccc3n3c4ccccc4nc3c2c2ccccc12] | 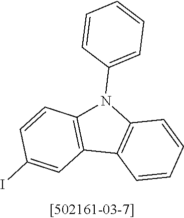 [SMILES: Ic1ccc2c(c1)c1ccccc1n2-c1ccccc1] | 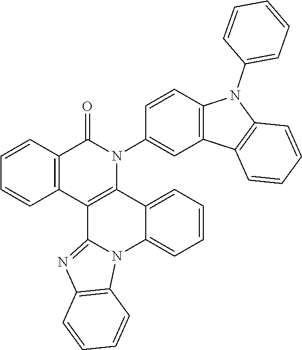 [SMILES: O=c1c2ccccc2c2c(c3ccccc3n3c4ccccc4nc23)n1-c1ccc2c(c1)c1ccccc1n2-c1ccccc1] | 74% |
| 4a | 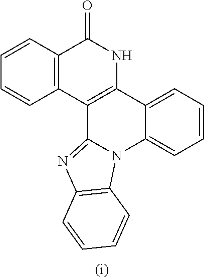 [SMILES: O=c1[nH]c2c3ccccc3n3c4ccccc4nc3c2c2ccccc12] | 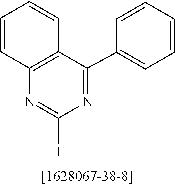 [SMILES: Ic1nc(-c2ccccc2)c2ccccc2n1] | 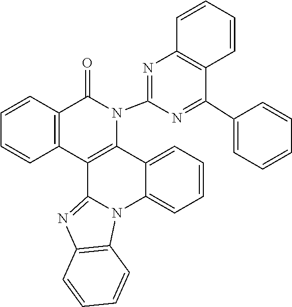 [SMILES: O=c1c2ccccc2c2c(c3ccccc3n3c4ccccc4nc23)n1-c1nc(-c2ccccc2)c2ccccc2n1] | 61% |
| 5a | 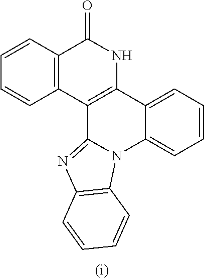 [SMILES: O=c1[nH]c2c3ccccc3n3c4ccccc4nc3c2c2ccccc12] | 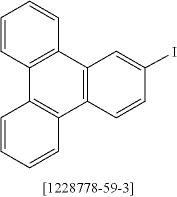 [SMILES: Ic1ccc2c3ccccc3c3ccccc3c2c1] | 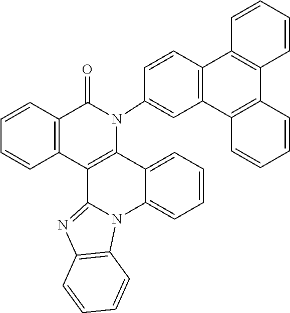 [SMILES: O=c1c2ccccc2c2c(c3ccccc3n3c4ccccc4nc23)n1-c1ccc2c3ccccc3c3ccccc3c2c1] | 79% |
| 6a | 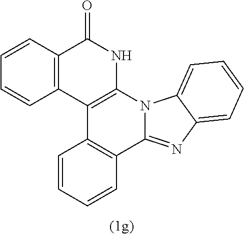 [SMILES: O=c1[nH]c2c(c3ccccc13)c1ccccc1c1nc3ccccc3n12] | 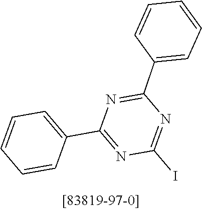 [SMILES: Ic1nc(-c2ccccc2)nc(-c2ccccc2)n1] | 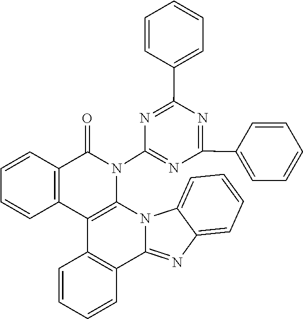 [SMILES: O=c1c2ccccc2c2c3ccccc3c3nc4ccccc4n3c2n1-c1nc(-c2ccccc2)nc(-c2ccccc2)n1] | 70% |
| 7a | 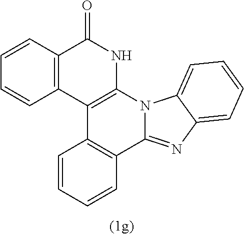 [SMILES: O=c1[nH]c2c(c3ccccc13)c1ccccc1c1nc3ccccc3n12] | 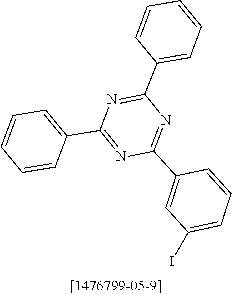 [SMILES: Ic1cccc(-c2nc(-c3ccccc3)nc(-c3ccccc3)n2)c1] | 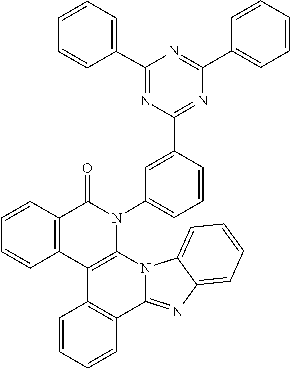 [SMILES: O=c1c2ccccc2c2c3ccccc3c3nc4ccccc4n3c2n1-c1cccc(-c2nc(-c3ccccc3)nc(-c3ccccc3)n2)c1] | 66% |
| 8a | 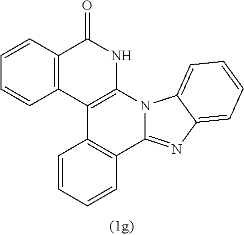 [SMILES: O=c1[nH]c2c(c3ccccc13)c1ccccc1c1nc3ccccc3n12] | 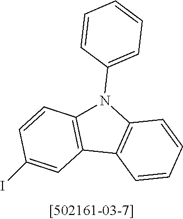 [SMILES: Ic1ccc2c(c1)c1ccccc1n2-c1ccccc1] | 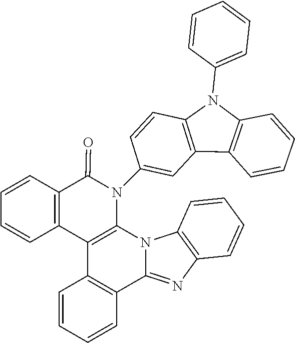 [SMILES: O=c1c2ccccc2c2c3ccccc3c3nc4ccccc4n3c2n1-c1ccc2c(c1)c1ccccc1n2-c1ccccc1] | 71% |

### b) 3-(2-Fluorophenyl)-4-oxoquinazoline-2-carbaldehyde

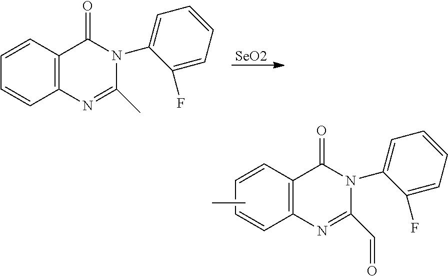

25.4 g (100 mmol) of 2-(2-fluorophenyl)-3-methylisoquinolin-1-one is dissolved in 500 ml of hot dioxane, and then 12 g of pulverized selenium dioxide (200 mmol) is added in portions while stirring. On completion of addition, the reaction mixture is heated under reflux for 8 h. Then the reaction mixture is filtered, ice-cold water is added to the solution, and the product is filtered off and recrystallized in toluene.

The yield is 18 g (73 mmol), corresponding to 70% of theory.

The following compounds are prepared in an analogous manner:

| Ex. | Reactant 1 | Product | Yield |
|---|---|---|---|
| 1b | 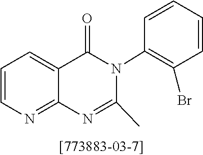 [SMILES: Cc1nc2ncccc2c(=O)n1-c1ccccc1Br] | 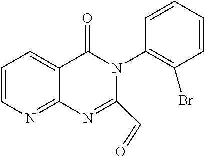 [SMILES: O=Cc1nc2ncccc2c(=O)n1-c1ccccc1Br] | 65% |

### c) 2,6-Diphenyl-8-(2-phenylphenyl)-9H-purine

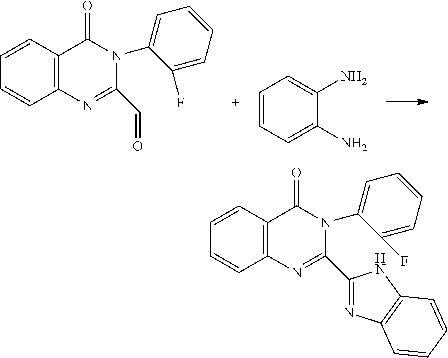

A solution of 7.5 g (28 mmol) of 3-(2-fluorophenyl)-4-oxoquinazoline-2-carbaldehyde and 3 g (28 mmol) of phenylene-1,2-diamine in 50 ml of DMF is heated to 80° C. The reaction mixture is left to stir for 8 h, and the resultant solution is brought to room temperature and then extracted with ethyl acetate (EtOAc). The organic layer is washed with salt solution, dried over anhydrous Na₂SO₄, and concentrated under reduced pressure. The crude product is purified by silica gel column chromatography with n-hexane-EtOAc. Yield: 7.7 (21 mmol), 78% of theory.

The following compounds can be obtained analogously:

| Ex. | Reactant 1 | Reactant 2 | Product | Yield |
|---|---|---|---|---|
| 1c | 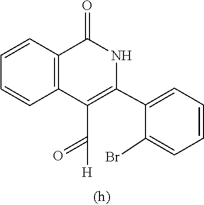 [SMILES: O=Cc1c(-c2ccccc2Br)[nH]c(=O)c2ccccc12] | 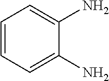 | 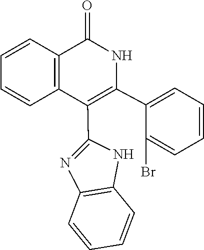 [SMILES: O=c1[nH]c(-c2ccccc2Br)c(-c2nc3ccccc3[nH]2)c2ccccc12] | 73% |
| 2c | 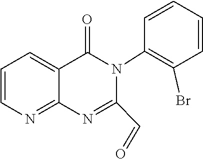 [SMILES: O=Cc1nc2ncccc2c(=O)n1-c1ccccc1Br] | 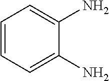 | 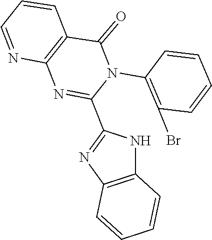 [SMILES: O=c1c2cccnc2nc(-c2nc3ccccc3[nH]2)n1-c1ccccc1Br] | 60% |

### d) 3-(1H-Benzimidazol-2-yl)-2-(2-fluorophenyl)quinazolin-4-one

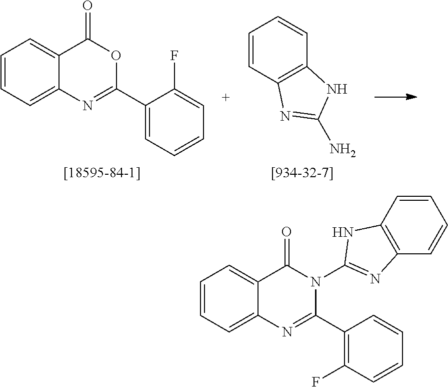 [SMILES: O=c1oc(-c2ccccc2F)nc2ccccc12 ; Nc1nc2ccccc2[nH]1 ; O=c1c2ccccc2nc(-c2ccccc2F)n1-c1nc2ccccc2[nH]1]

A mixture of 4.8 g (20 mmol) of 2-(2-fluorophenyl)-3,1-benzoxazin-4-one and 2.66 g (20 mmol) of 2-aminobenzimidazole is fused together in an oil bath at 200-250° C. for 30 minutes, in such a way that the reaction mixture is not to evaporate off owing to high heating. The mixture is cooled down, 20 ml of ethanol is added, and the mixture is filtered. The solids separated off are washed with ethanol, dried and recrystallized repeatedly with ethanol in order to obtain the pure product. Yield: 4.6 (13 mmol), 65% of theory.

The following compounds can be obtained analogously:

| Ex. | Reactant 1 | Reactant 2 | Product | Yield |
|---|---|---|---|---|
| 1d | 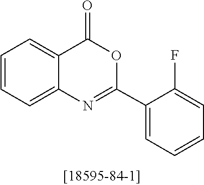 [SMILES: O=c1oc(-c2ccccc2F)nc2ccccc12] | 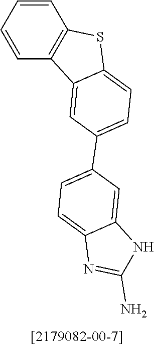 [SMILES: Nc1nc2ccc(-c3ccc4sc5ccccc5c4c3)cc2[nH]1] | 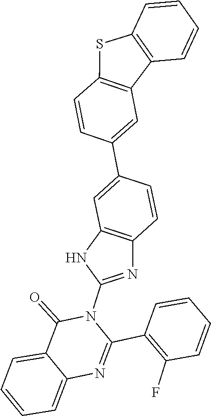 [SMILES: O=c1c2ccccc2nc(-c2ccccc2F)n1-c1nc2ccc(-c3ccc4sc5ccccc5c4c3)cc2[nH]1] | 57% |
| 2d | 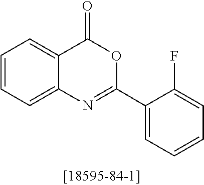 [SMILES: O=c1oc(-c2ccccc2F)nc2ccccc12] | 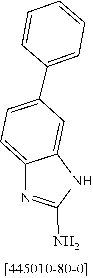 [SMILES: Nc1nc2ccc(-c3ccccc3)cc2[nH]1] | 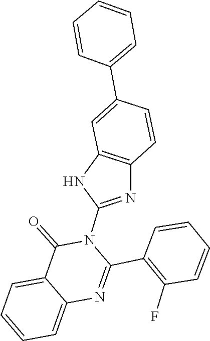 [SMILES: O=c1c2ccccc2nc(-c2ccccc2F)n1-c1nc2ccc(-c3ccccc3)cc2[nH]1] | 61% |
| 3d | 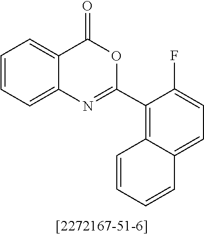 [SMILES: O=c1oc(-c2c(F)ccc3ccccc23)nc2ccccc12] | 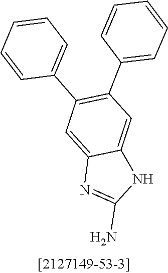 [SMILES: Nc1nc2cc(-c3ccccc3)c(-c3ccccc3)cc2[nH]1] | 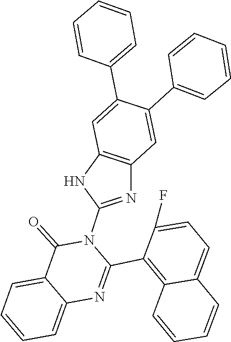 [SMILES: O=c1c2ccccc2nc(-c2c(F)ccc3ccccc23)n1-c1nc2cc(-c3ccccc3)c(-c3ccccc3)cc2[nH]1] | 60% |
| 4d | 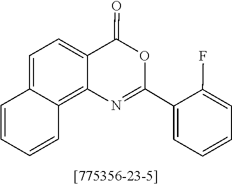 [SMILES: O=c1oc(-c2ccccc2F)nc2c1ccc1ccccc12] | 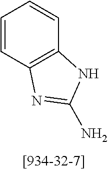 | 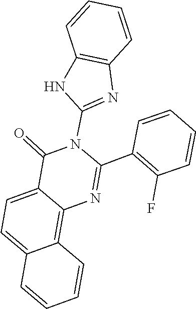 [SMILES: O=c1c2ccc3ccccc3c2nc(-c2ccccc2F)n1-c1nc2ccccc2[nH]1] | 53% |

### e) Cyclization

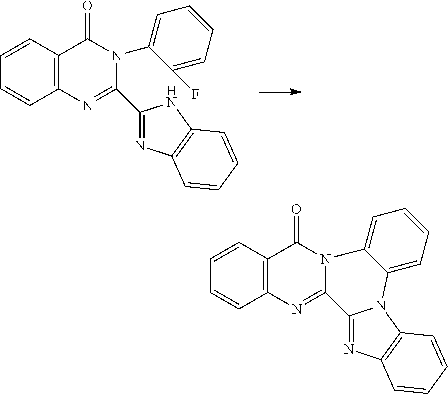 [SMILES: O=c1c2ccccc2nc(-c2nc3ccccc3[nH]2)n1-c1ccccc1F ; O=c1c2ccccc2nc2c3nc4ccccc4n3c3ccccc3n12]

To a solution of 1.78 g (5 mmol) of 2,6-diphenyl-8-(2-phenylphenyl)-9H-purine in 50 ml of DMF is added 0.32 g (1 mmol) of Cs₂CO₃, and the reaction mixture is heated at 150° C. in a microwave for 10 min. Once the microwave vial has cooled down to room temperature, the solvent is diluted with EtOAc (150 ml) and washed with saturated NaCl solution (2×100 ml). The organic phase is separated and dried over Mg₂SO₄. The residue is purified by column chromatography using an ethyl acetate/hexane gradient (0-60%). The residue is recrystallized from toluene and finally under high vacuum (p=5×10⁻⁵ mbar). The purity is 99.9%.

Yield: 1.3 (3.9 mmol), 78% of theory.

The following compounds can be obtained analogously:

| Ex. | Reactant 1 | Product | Yield |
|---|---|---|---|
| 1e | 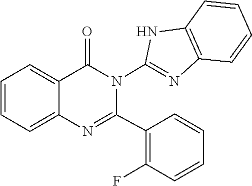 [SMILES: O=c1c2ccccc2nc(-c2ccccc2F)n1-c1nc2ccccc2[nH]1] | 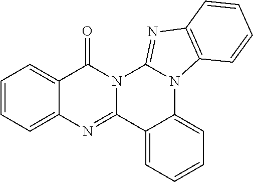 [SMILES: O=c1c2ccccc2nc2c3ccccc3n3c4ccccc4nc3n12] | 69% |
| 2e | 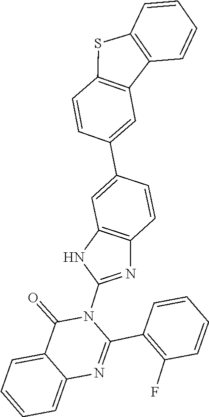 [SMILES: O=c1c2ccccc2nc(-c2ccccc2F)n1-c1nc2ccc(-c3ccc4sc5ccccc5c4c3)cc2[nH]1] | 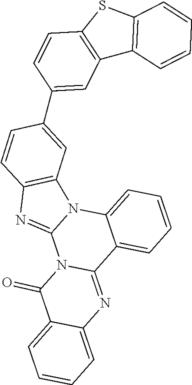 [SMILES: O=c1c2ccccc2nc2c3ccccc3n3c4cc(-c5ccc6sc7ccccc7c6c5)ccc4nc3n12] | 74% |
| 3e | 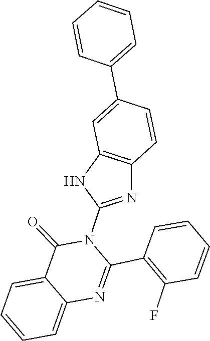 [SMILES: O=c1c2ccccc2nc(-c2ccccc2F)n1-c1nc2ccc(-c3ccccc3)cc2[nH]1] | 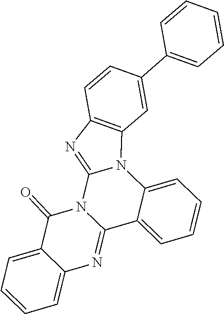 [SMILES: O=c1c2ccccc2nc2c3ccccc3n3c4cc(-c5ccccc5)ccc4nc3n12] | 71% |
| 4e | 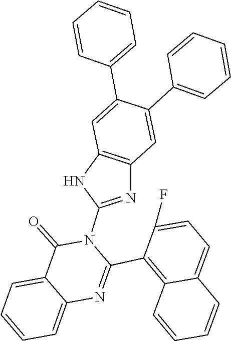 [SMILES: O=c1c2ccccc2nc(-c2c(F)ccc3ccccc23)n1-c1nc2cc(-c3ccccc3)c(-c3ccccc3)cc2[nH]1] | 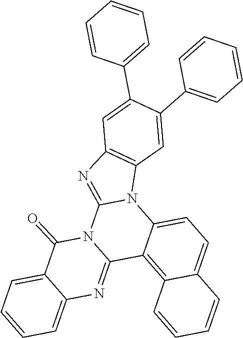 [SMILES: O=c1c2ccccc2nc2c3c4ccccc4ccc3n3c4cc(-c5ccccc5)c(-c5ccccc5)cc4nc3n12] | 68% |

### f) Cyclization

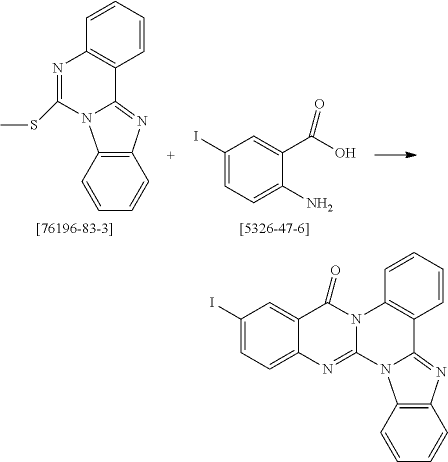 [SMILES: CSc1nc2ccccc2c2nc3ccccc3n12 ; Nc1ccc(I)cc1C(=O)O ; O=c1c2cc(I)ccc2nc2n1c1ccccc1c1nc3ccccc3n12]

A mixture of 2 g (7.5 mmol) of 6-(methylthio)benzimidazo[1,2-c]quinazoline, 3.74 g (43 mmol) of anthranilic acid and 20 g of graphite in a 70 ml quartz vial is introduced into a microwave oven. Irradiation is programmed at 105 W for 90 min. After a period of 2±3 min., the mixture reaches 170° C. and stays constant at that temperature. After cooling, the graphite powder is filtered, reacted with 10 ml of dichloromethane, filtered, and washed with 5 ml of dichloromethane. The organic solution is washed with a saturated sodium bicarbonate solution, and the crude product is recrystallized in ethanol.

Yield: 3.0 g (6.64 mmol), 88% of theory.

The following compounds can be obtained analogously:

| Ex. | Reactant 1 | Reactant 2 | Product | Yield |
|---|---|---|---|---|
| 1f | 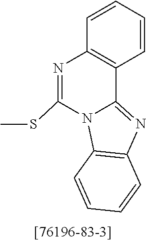 [SMILES: CSc1nc2ccccc2c2nc3ccccc3n12] | 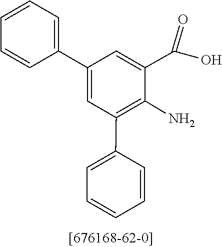 [SMILES: Nc1c(C(=O)O)cc(-c2ccccc2)cc1-c1ccccc1] | 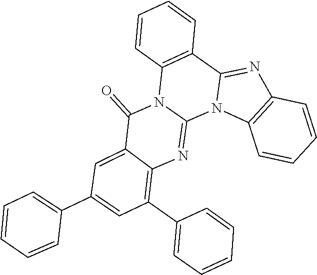 [SMILES: O=c1c2cc(-c3ccccc3)cc(-c3ccccc3)c2nc2n1c1ccccc1c1nc3ccccc3n12] | 89% |
| 2f | 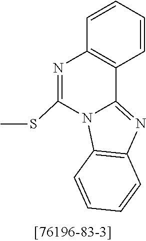 [SMILES: CSc1nc2ccccc2c2nc3ccccc3n12] | 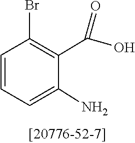 [SMILES: Nc1cccc(Br)c1C(=O)O] | 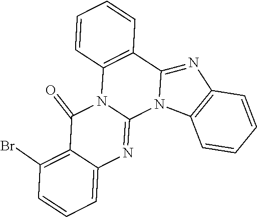 [SMILES: O=c1c2c(Br)cccc2nc2n1c1ccccc1c1nc3ccccc3n12] | 87% |
| 3f | 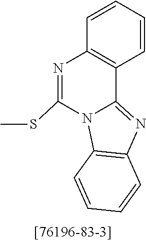 [SMILES: CSc1nc2ccccc2c2nc3ccccc3n12] | 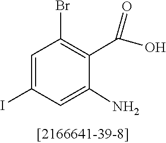 [SMILES: Nc1cc(I)cc(Br)c1C(=O)O] | 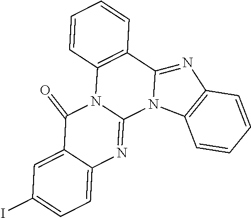 [SMILES: O=c1c2cc(I)ccc2nc2n1c1ccccc1c1nc3ccccc3n12] | 64% |
| 4f | 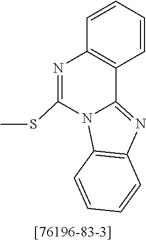 [SMILES: CSc1nc2ccccc2c2nc3ccccc3n12] | 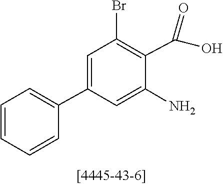 [SMILES: Nc1cc(-c2ccccc2)cc(Br)c1C(=O)O] | 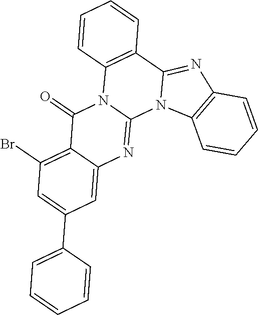 [SMILES: O=c1c2c(Br)cc(-c3ccccc3)cc2nc2n1c1ccccc1c1nc3ccccc3n12] | 61% |
| 5f | 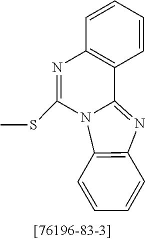 [SMILES: CSc1nc2ccccc2c2nc3ccccc3n12] | 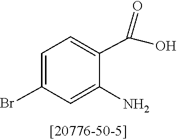 [SMILES: Nc1cc(Br)ccc1C(=O)O] | 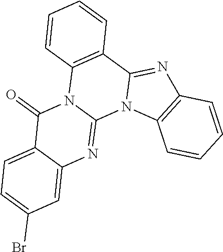 [SMILES: O=c1c2ccc(Br)cc2nc2n1c1ccccc1c1nc3ccccc3n12] | 72% |
| 6f | 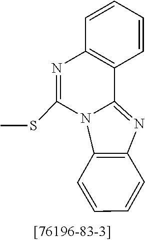 [SMILES: CSc1nc2ccccc2c2nc3ccccc3n12] | 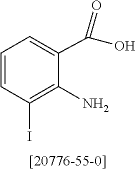 [SMILES: Nc1c(I)cccc1C(=O)O] | 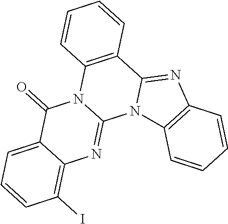 [SMILES: O=c1c2cccc(I)c2nc2n1c1ccccc1c1nc3ccccc3n12] | 70% |
| 7f | 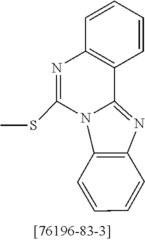 [SMILES: CSc1nc2ccccc2c2nc3ccccc3n12] | 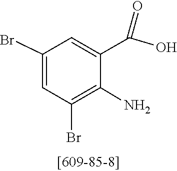 [SMILES: Nc1c(Br)cc(Br)cc1C(=O)O] | 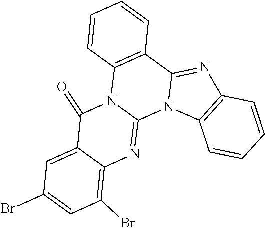 [SMILES: O=c1c2cc(Br)cc(Br)c2nc2n1c1ccccc1c1nc3ccccc3n12] | 58% |

### g) Cyclization

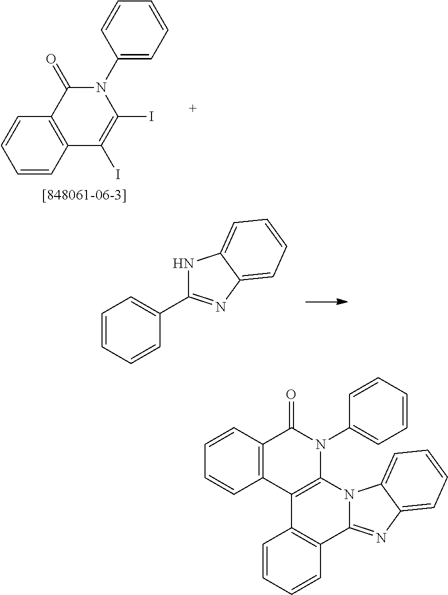 [SMILES: O=c1c2ccccc2c(I)c(I)n1-c1ccccc1 ; c1ccc(-c2nc3ccccc3[nH]2)cc1 ; O=c1c2ccccc2c2c3ccccc3c3nc4ccccc4n3c2n1-c1ccccc1]

Under protective gas, 3.9 g (20 mmol, 1.0 eq.) of 2-phenylbenzimidazole, 14.1 g (30 mmol, 1.5 eq.) of 3,4-diiodo-2-phenyl-1(2H)-isoquinolinone and 8.2 g (60 mmol, 3.0 eq.) of K₂CO₃, 0.5 (2 mmol) of Pd(OAc)₂ and finally 1.9 g (4 mmol) of Xphos are added to 200 ml of DMF. The mixture is heated at 160° C. for 80 h and then comes to room temperature. This is followed by quenching for 5 min with water and dilution with 8 ml of ethyl acetate. The organic phase is separated and concentrated under reduced pressure. The crude product is then separated by flash chromatography on silica gel (3-10% ethyl acetate/petroleum ether).

Yield: 2.9 g (18.9 mmol), 63% of theory

The following compounds can be obtained analogously:

| Ex. | Reactant 1 | Reactant 2 | Product | Yield |
|---|---|---|---|---|
| 1g | 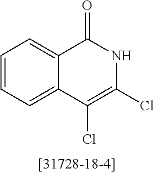 [SMILES: O=c1[nH]c(Cl)c(Cl)c2ccccc12] | 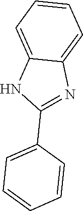 [SMILES: c1ccc(-c2nc3ccccc3[nH]2)cc1] | 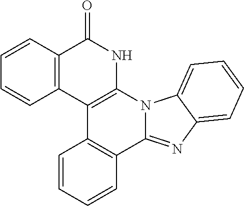 [SMILES: O=c1[nH]c2c(c3ccccc13)c1ccccc1c1nc3ccccc3n12] | 32% |
| 2g | 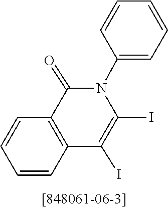 [SMILES: O=c1c2ccccc2c(I)c(I)n1-c1ccccc1] | 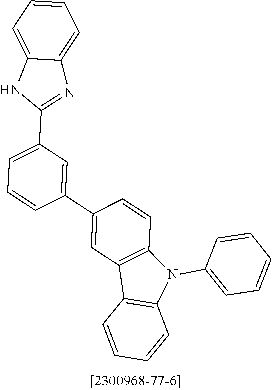 [SMILES: c1ccc(-n2c3ccccc3c3cc(-c4cccc(-c5nc6ccccc6[nH]5)c4)ccc32)cc1] | 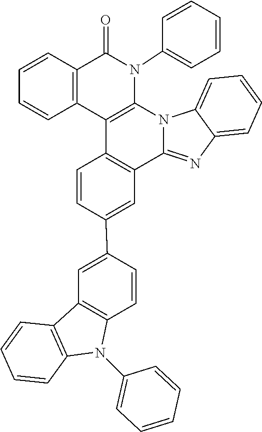 [SMILES: O=c1c2ccccc2c2c3ccc(-c4ccc5c(c4)c4ccccc4n5-c4ccccc4)cc3c3nc4ccccc4n3c2n1-c1ccccc1] | 37% |
| 3g | 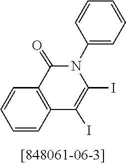 [SMILES: O=c1c2ccccc2c(I)c(I)n1-c1ccccc1] | 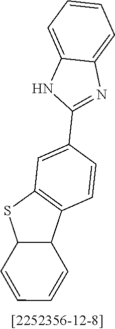 [SMILES: C1=CC2Sc3cc(-c4nc5ccccc5[nH]4)ccc3C2C=C1] | 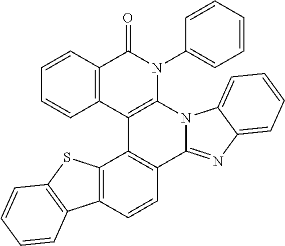 [SMILES: O=c1c2ccccc2c2c3c(ccc4c5ccccc5sc43)c3nc4ccccc4n3c2n1-c1ccccc1] | 41% |
| 4g | 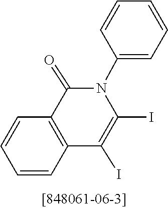 [SMILES: O=c1c2ccccc2c(I)c(I)n1-c1ccccc1] | 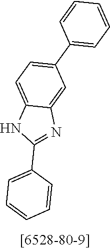 [SMILES: c1ccc(-c2ccc3[nH]c(-c4ccccc4)nc3c2)cc1] | 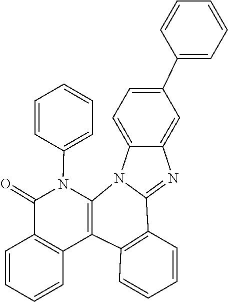 [SMILES: O=c1c2ccccc2c2c3ccccc3c3nc4cc(-c5ccccc5)ccc4n3c2n1-c1ccccc1] | 55% |

### h) 3-(2-Bromophenyl)-1-oxo-2H-isoquinoline-4-carbaldehyde

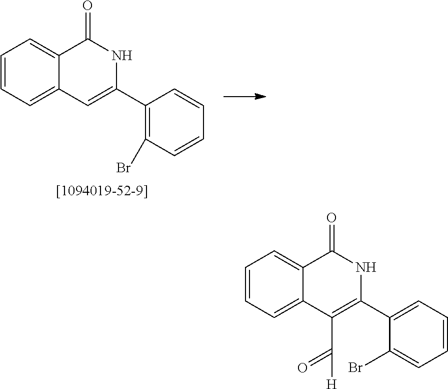 [SMILES: O=c1[nH]c(-c2ccccc2Br)cc2ccccc12 ; O=Cc1c(-c2ccccc2Br)[nH]c(=O)c2ccccc12]

9.7 g (63.6 mmol) of phosphorus oxychloride is added to 40 ml of dimethylformamide, and the mixture is cooled in an ice bath. The flask is stirred for 4 hours. The resulting mixture is stirred at the same temperature for 4 h. An ice-cold solution of 3.2 g (12.7 mmol) of 3-(2-bromophenyl)-2H-isoquinolin-1-one in DMF (10 ml) is added dropwise to the mixture and, after the addition has ended, warmed gradually to 60° C. and stirred for 20 hours. The reaction mixture is cooled in an ice bath, 50 ml of ice-cold water is added cautiously, and the mixture is basified with 35% NaOH solution. The solids are filtered off. The filtrate is extracted with ethyl acetate. The combined ethyl acetate extracts are washed with water and salt solution, dried over anhydrous sodium sulfate and then concentrated. The residue is recrystallized from methanol.

Yield: 2.8 g (8.5 mmol), 80% of theory

### i) Cyclization

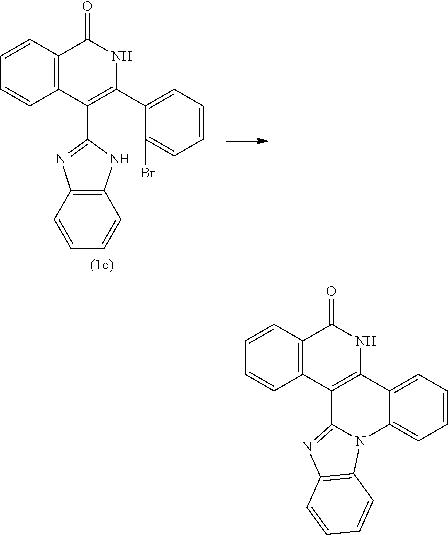 [SMILES: O=c1[nH]c(-c2ccccc2Br)c(-c2nc3ccccc3[nH]2)c2ccccc12 ; O=c1[nH]c2c3ccccc3n3c4ccccc4nc3c2c2ccccc12]

Under protective gas, 16.6 g (40 mmol, 1.0 eq.) of compound 1c, 16.4 g (120 mmol, 3.0 eq.) of K2C03, 1 g (4 mmol) of Pd(OAc)₂ and finally 3.8 g (8 mmol) of Xphos are added to 400 ml of DMF. The mixture is heated at 160° C. for 80 h and then comes to room temperature. This is followed by quenching with 20 ml of water and dilution with 20 ml of ethyl acetate. The organic phase is separated and concentrated under reduced pressure. The crude product is then separated by flash chromatography on silica gel (3-10% ethyl acetate/petroleum ether).

Yield: 8 g (22 mmol), 75% of theory

The following compounds can be obtained analogously:

| Ex. | Reactant 1 | Product | Yield |
|---|---|---|---|
| 1i | 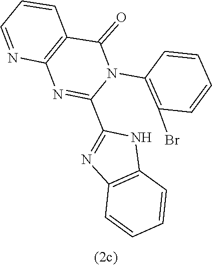 [SMILES: O=c1c2cccnc2nc(-c2nc3ccccc3[nH]2)n1-c1ccccc1Br] | 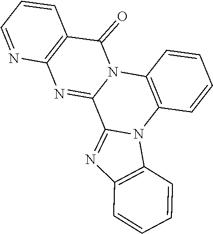 [SMILES: O=c1c2cccnc2nc2c3nc4ccccc4n3c3ccccc3n12] | 65% |

### j) Suzuki Reaction

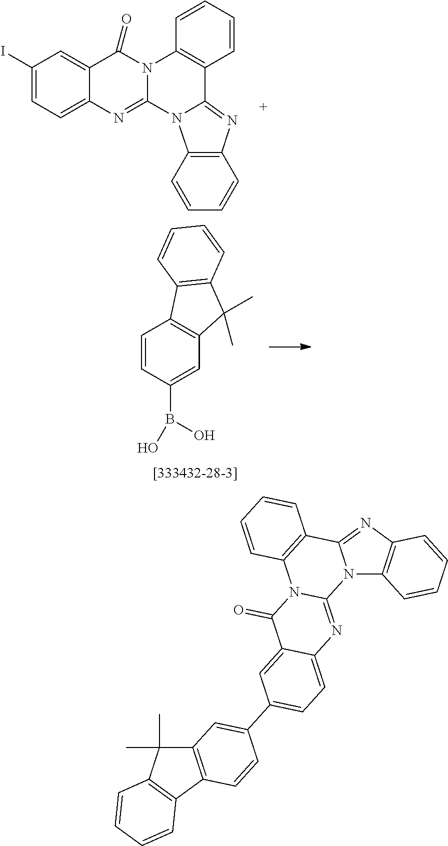

53 g (155 mmol) of compound f, 41 g (172 mmol) of 9,9-dimethyl-9H-fluorene-2-boronic acid and 36 g (340 mmol) of sodium carbonate are suspended in 1000 ml of ethylene glycol diamine ether and 280 ml of water. To this suspension is added 1.8 g (1.5 mmol) of tetrakis(triphenylphosphine)palladium(0), and the reaction mixture is heated under reflux for 16 h. After cooling, the organic phase is removed, filtered through silica gel and then concentrated to dryness. The product is purified via column chromatography on silica gel with toluene/heptane (1:2) and finally sublimed under high vacuum (p=5×10⁻⁷ mbar) (99.9% purity). The yield is 42 g (80 mmol), corresponding to 70% of theory.

The following compounds are prepared in an analogous manner:

| Ex. | Reactant 1 | Reactant 2 | Product | Yield |
|---|---|---|---|---|
| 1j | 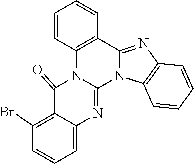 [SMILES: O=c1c2c(Br)cccc2nc2n1c1ccccc1c1nc3ccccc3n12] | 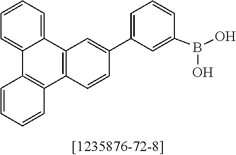 [SMILES: OB(O)c1cccc(-c2ccc3c4ccccc4c4ccccc4c3c2)c1] | 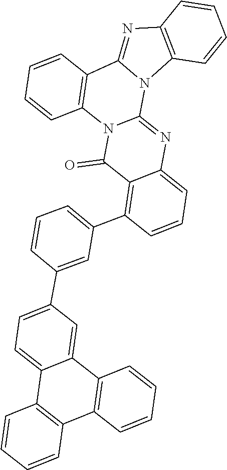 [SMILES: O=c1c2c(-c3cccc(-c4ccc5c6ccccc6c6ccccc6c5c4)c3)cccc2nc2n1c1ccccc1c1nc3ccccc3n12] | 73% |
| 2j | 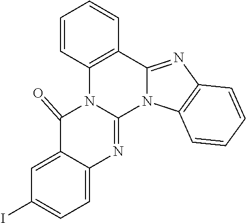 [SMILES: O=c1c2cc(I)ccc2nc2n1c1ccccc1c1nc3ccccc3n12] | 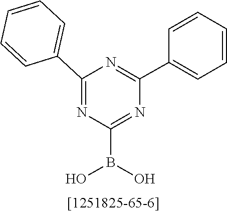 [SMILES: OB(O)c1nc(-c2ccccc2)nc(-c2ccccc2)n1] | 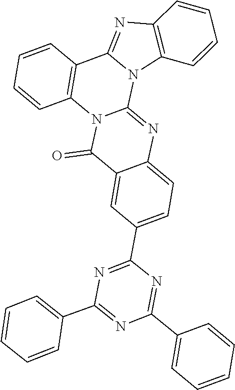 [SMILES: O=c1c2cc(-c3nc(-c4ccccc4)nc(-c4ccccc4)n3)ccc2nc2n1c1ccccc1c1nc3ccccc3n12] | 84% |
| 3j | 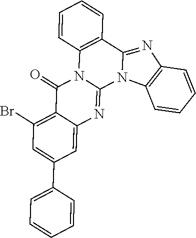 [SMILES: O=c1c2c(Br)cc(-c3ccccc3)cc2nc2n1c1ccccc1c1nc3ccccc3n12] | 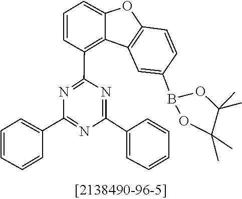 [SMILES: CC1(C)OB(c2ccc3oc4cccc(-c5nc(-c6ccccc6)nc(-c6ccccc6)n5)c4c3c2)OC1(C)C] | 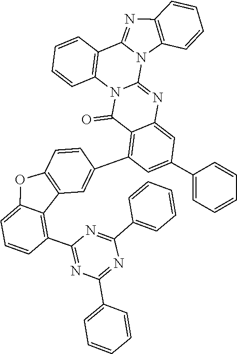 [SMILES: O=c1c2c(-c3ccc4oc5cccc(-c6nc(-c7ccccc7)nc(-c7ccccc7)n6)c5c4c3)cc(-c3ccccc3)cc2nc2n1c1ccccc1c1nc3ccccc3n12] | 72% |
| 4j | 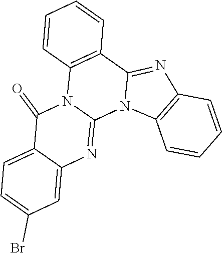 [SMILES: O=c1c2ccc(Br)cc2nc2n1c1ccccc1c1nc3ccccc3n12] | 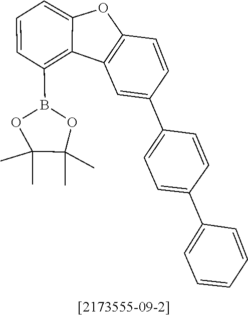 [SMILES: CC1(C)OB(c2cccc3oc4ccc(-c5ccc(-c6ccccc6)cc5)cc4c23)OC1(C)C] | 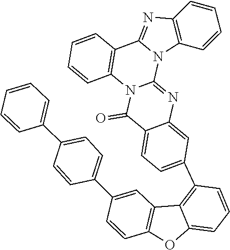 [SMILES: O=c1c2ccc(-c3cccc4oc5ccc(-c6ccc(-c7ccccc7)cc6)cc5c34)cc2nc2n1c1ccccc1c1nc3ccccc3n12] | 79% |
| 5j | 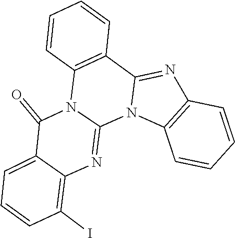 [SMILES: O=c1c2cccc(I)c2nc2n1c1ccccc1c1nc3ccccc3n12] | 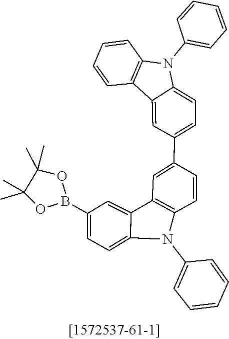 [SMILES: CC1(C)OB(c2ccc3c(c2)c2cc(-c4ccc5c(c4)c4ccccc4n5-c4ccccc4)ccc2n3-c2ccccc2)OC1(C)C] | 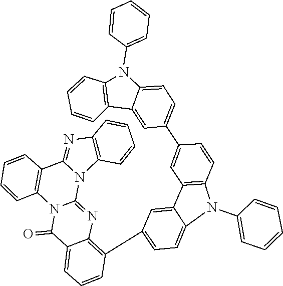 | 71% |
| 6j | 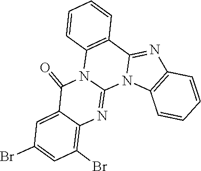 [SMILES: O=c1c2cc(Br)cc(Br)c2nc2n1c1ccccc1c1nc3ccccc3n12] | 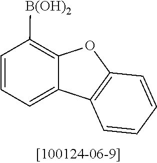 [SMILES: OB(O)c1cccc2c1oc1ccccc12] | 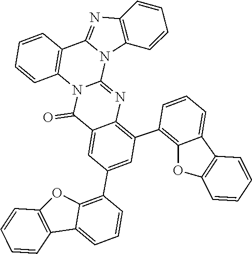 [SMILES: O=c1c2cc(-c3cccc4c3oc3ccccc34)cc(-c3cccc4c3oc3ccccc34)c2nc2n1c1ccccc1c1nc3ccccc3n12] | 69% |
| 7j | 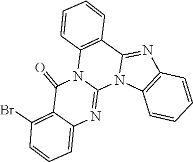 [SMILES: O=c1c2c(Br)cccc2nc2n1c1ccccc1c1nc3ccccc3n12] | 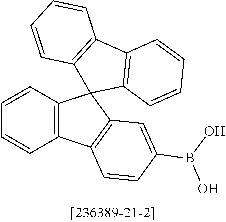 [SMILES: OB(O)c1ccc2c(c1)C1(c3ccccc3-c3ccccc31)c1ccccc1-2] | 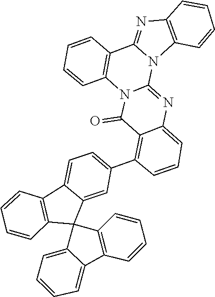 [SMILES: O=c1c2c(-c3ccc4c(c3)C3(c5ccccc5-c5ccccc53)c3ccccc3-4)cccc2nc2n1c1ccccc1c1nc3ccccc3n12] | 71% |
| 8j | 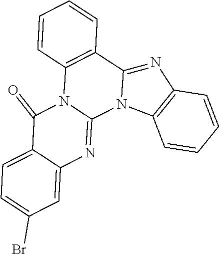 [SMILES: O=c1c2ccc(Br)cc2nc2n1c1ccccc1c1nc3ccccc3n12] | 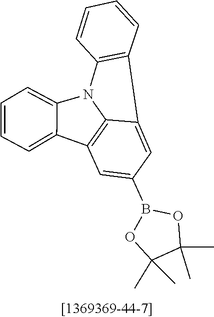 [SMILES: CC1(C)OB(c2cc3c4ccccc4n4c5ccccc5c(c2)c34)OC1(C)C] | 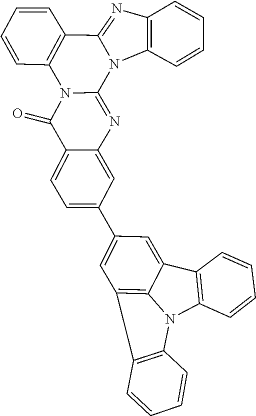 [SMILES: O=c1c2ccc(-c3cc4c5ccccc5n5c6ccccc6c(c3)c45)cc2nc2n1c1ccccc1c1nc3ccccc3n12] | 80% |

### Production of the Electroluminescent Devices

Examples E1 to E16 which follow (see table 1) present the use of the materials of the invention in OLEDs.

Pretreatment for Examples E1 to E16: Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as EG1:IC2:TEG1 (49%:44%:7%) mean here that the material EG1 is present in the layer in a proportion by volume of 49%, IC2 in a proportion of 44% and TEG1 in a proportion of 7%. Analogously, the electron transport layer may also consist of a mixture of two materials.

The OLEDs are characterized in a standard manner. For this purpose, electroluminescence spectra, current efficiency (CE, measured in cd/A) and external quantum efficiency (EQE, measured in %) are determined as a function of luminance, calculated from current-voltage-luminance characteristics assuming Lambertian emission characteristics. The parameter U1000 refers to the voltage which is required for a luminance of 1000 cd/m². CE1000 and EQE1000 respectively denote the current efficiency and external quantum efficiency that are attained at 1000 cd/m². Electroluminescence spectra are determined at a luminance of 1000 cd/m², and these are used to calculate the CIE 1931 x and y color coordinates. The results thus obtained can be found in table 3.

### Use of the Materials of the Invention in OLEDs

Inventive compound EG1 is used in example E1 as matrix material in the emission layer of red-phosphorescing OLEDs. Compounds EG2 to EG13 are used in examples E2 to E13 as matrix material in the emission layer of green-phosphorescing OLEDs. Inventive compounds EG3, EG7 and EG9 are used in examples E14 to E16 as electron transporter in the electron transport layer (ETL) of green-phosphorescing OLEDs.

In all cases, good performance data of the OLEDs are achieved (table 3).

TABLE 1
Structure of the electroluminescent devices

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | EG1:IC2:TER5 | ST2 | ST2:LiQ | LiQ |
| | 5 nm | 230 nm | 20 nm | (57%:40%:7%) | 5 nm | (50%:50%) | 1 nm |
| | | | | 35 nm | | 30 nm | |
| E2 | HATCN | SpMA1 | SpMA2 | EG2:IC2:TEG1 | ST2 | ST2:LiQ | LiQ |
| | 5 nm | 230 nm | 20 nm | (49%:44%:7%) | 5 nm | (50%:50%) | 1 nm |
| | | | | 40 nm | | 30 nm | |
| E3 | HATCN | SpMA1 | SpMA2 | EG3:IC2:TEG1 | ST2 | ST2:LiQ | LiQ |
| | 5 nm | 230 nm | 20 nm | (49%:44%:7%) | 5 nm | (50%:50%) | 1 nm |
| | | | | 40 nm | | 30 nm | |
| E4 | HATCN | SpMA1 | SpMA2 | EG4:IC2:TEG1 | ST2 | ST2:LiQ | LiQ |
| | 5 nm | 230 nm | 20 nm | (49%:44%:7%) | 5 nm | (50%:50%) | 1 nm |
| | | | | 40 nm | | 30 nm | |
| E5 | HATCN | SpMA1 | SpMA2 | EG5:IC3:TEG1 | ST2 | ST2:LiQ | LiQ |
| | 5 nm | 230 nm | 20 nm | (45%:45%:10%) | 10 nm | (50%:50%) | 1 nm |
| | | | | 30 nm | | 30 nm | |
| E6 | HATCN | SpMA1 | SpMA2 | EG6:IC3:TEG1 | ST2 | ST2:LiQ | LiQ |
| | 5 nm | 230 nm | 20 nm | (44%:44%:12%) | 10 nm | (50%:50%) | 1 nm |
| | | | | 30 nm | | 30 nm | |
| E7 | HATCN | SpMA1 | SpMA2 | EG7:IC3:TEG1 | ST2 | ST2:LiQ | LiQ |
| | 5 nm | 230 nm | 20 nm | (44%:44%:12%) | 10 nm | (50%:50%) | 1 nm |
| | | | | 30 nm | | 30 nm | |
| E8 | HATCN | SpMA1 | SpMA2 | EG8:IC3:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E9 | HATCN | SpMA1 | SpMA2 | EG9:IC3:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E10 | HATCN | SpMA1 | SpMA2 | EG10:IC3:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E11 | HATCN | SpMA1 | SpMA2 | EG11:IC3:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E12 | HATCN | SpMA1 | SpMA2 | EG12:IC3:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E13 | HATCN | SpMA1 | SpMA2 | EG13:IC1:TEG1 | ST2 | ST2:LiQ | LiQ |
| | 5 nm | 230 nm | 20 nm | (46%:47%:7%) | 10 nm | (50%:50%) | 1 nm |
| | | | | 30 nm | | 30 nm | |
| E14 | HATCN | SpMA1 | SpMA2 | IC1:TEG1 | — | EG3 | LiQ |
| | 5 nm | 70 nm | 15 nm | (90%:10%) | | 45 nm | 3 nm |
| | | | | 25 nm | | | |
| E15 | HATCN | SpMA1 | SpMA2 | IC1:TEG1 | — | EG7 | LiQ |
| | 5 nm | 70 nm | 15 nm | (90%:10%) | | 45 nm | 3 nm |
| | | | | 25 nm | | | |
| E16 | HATCN | SpMA1 | SpMA2 | IC1:TEG1 | — | EG9 | LiQ |
| | 5 nm | 70 nm | 15 nm | (90%:10%) | | 45 nm | 3 nm |
| | | | | 25 nm | | | |

TABLE 2
Structural formulae of the materials for the
electroluminescent devices

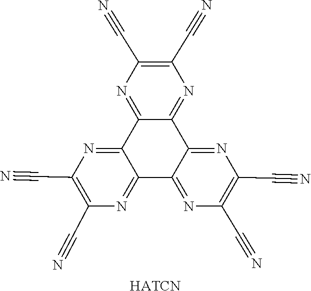

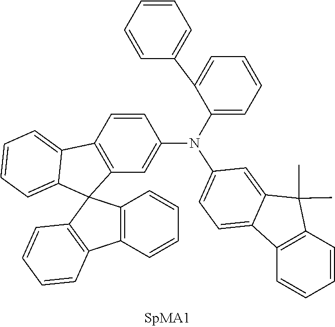 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

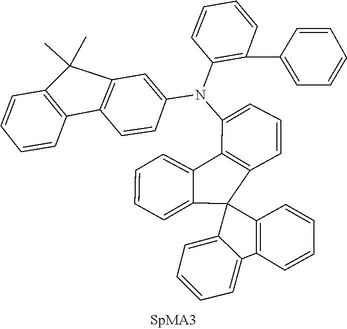 [SMILES: CC1(C)c2ccccc2-c2ccc(N(c3ccccc3-c3ccccc3)c3cccc4c3-c3ccccc3C43c4ccccc4-c4ccccc43)cc21]

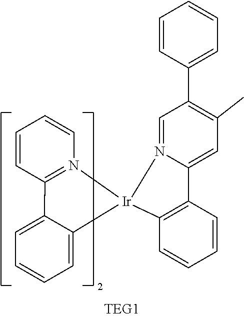

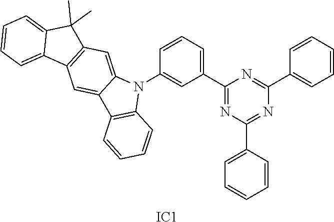 [SMILES: CC1(C)c2ccccc2-c2cc3c4ccccc4n(-c4cccc(-c5nc(-c6ccccc6)nc(-c6ccccc6)n5)c4)c3cc21 ; "IC1"]

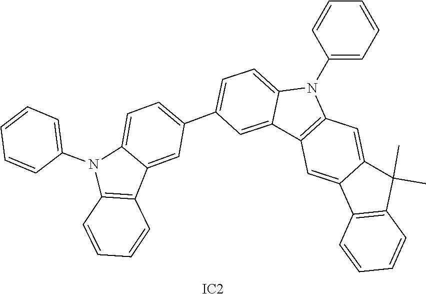 [SMILES: CC1(C)c2ccccc2-c2cc3c4cc(-c5ccc6c(c5)c5ccccc5n6-c5ccccc5)ccc4n(-c4ccccc4)c3cc21 ; "IC2"]

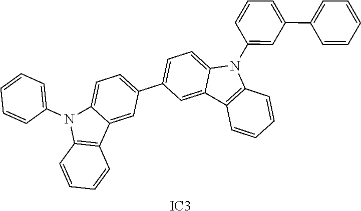

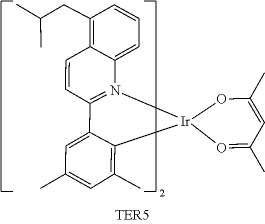

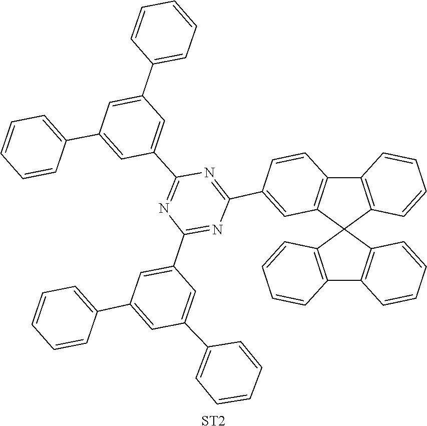

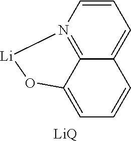

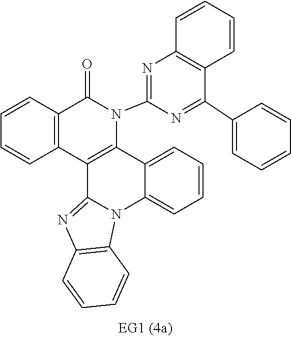 [SMILES: O=c1c2ccccc2c2c(c3ccccc3n3c4ccccc4nc23)n1-c1nc(-c2ccccc2)c2ccccc2n1]

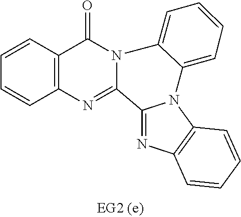 [SMILES: O=c1c2ccccc2nc2c3nc4ccccc4n3c3ccccc3n12]

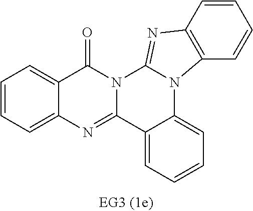 [SMILES: O=c1c2ccccc2nc2c3ccccc3n3c4ccccc4nc3n12]

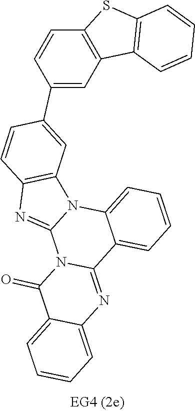 [SMILES: O=c1c2ccccc2nc2c3ccccc3n3c4cc(-c5ccc6sc7ccccc7c6c5)ccc4nc3n12]

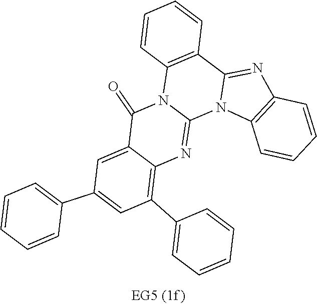 [SMILES: O=c1c2cc(-c3ccccc3)cc(-c3ccccc3)c2nc2n1c1ccccc1c1nc3ccccc3n12]

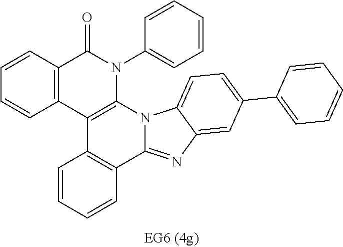 [SMILES: O=c1c2ccccc2c2c3ccccc3c3nc4cc(-c5ccccc5)ccc4n3c2n1-c1ccccc1]

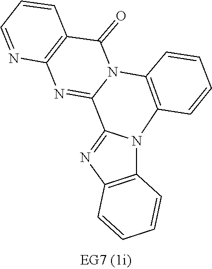 [SMILES: O=c1c2cccnc2nc2c3nc4ccccc4n3c3ccccc3n12]

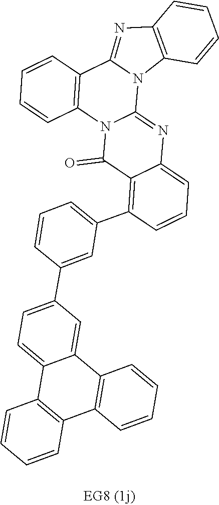 [SMILES: O=c1c2c(-c3cccc(-c4ccc5c6ccccc6c6ccccc6c5c4)c3)cccc2nc2n1c1ccccc1c1nc3ccccc3n12]

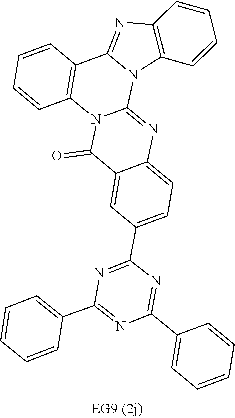 [SMILES: O=c1c2cc(-c3nc(-c4ccccc4)nc(-c4ccccc4)n3)ccc2nc2n1c1ccccc1c1nc3ccccc3n12]

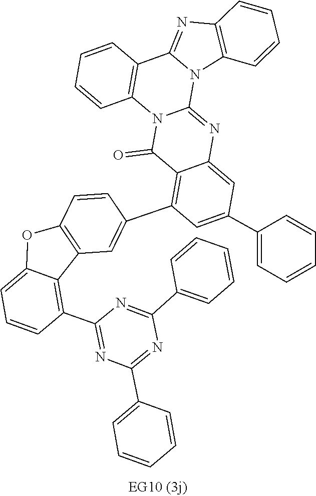 [SMILES: O=c1c2c(-c3ccc4oc5cccc(-c6nc(-c7ccccc7)nc(-c7ccccc7)n6)c5c4c3)cc(-c3ccccc3)cc2nc2n1c1ccccc1c1nc3ccccc3n12]

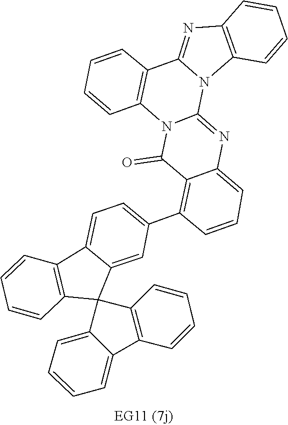 [SMILES: O=c1c2c(-c3ccc4c(c3)C3(c5ccccc5-c5ccccc53)c3ccccc3-4)cccc2nc2n1c1ccccc1c1nc3ccccc3n12]

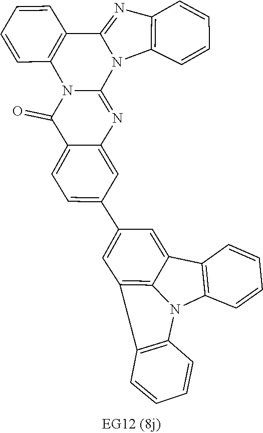 [SMILES: O=c1c2ccc(-c3cc4c5ccccc5n5c6ccccc6c(c3)c45)cc2nc2n1c1ccccc1c1nc3ccccc3n12]

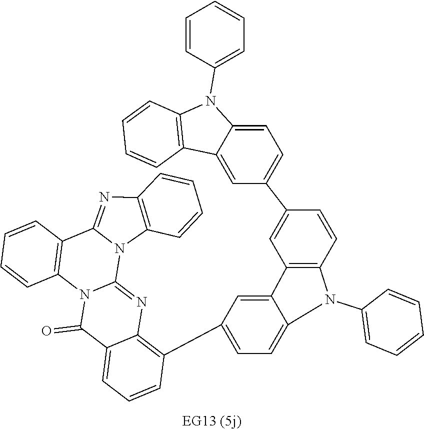 [SMILES: O=c1c2cccc(-c3ccc4c(c3)c3cc(-c5ccc6c(c5)c5ccccc5n6-c5ccccc5)ccc3n4-c3ccccc3)c2nc2n1c1ccccc1c1nc3ccccc3n12]

The figure between brackets for the respective compound in table 2 relates to the synthesis example.

TABLE 3
Performance data of the electroluminescent devices

| | U1000 | SE1000 | EQE 1000 | CIE x/y at |
|---|---|---|---|---|
| Ex. | (V) | (cd/A) | (%) | 1000 cd/m² |
| E1 | 3.7 | 23 | 21 | 0.66/0.34 |
| E2 | 4.1 | 67 | 16 | 0.33/0.62 |
| E3 | 3.9 | 65 | 17 | 0.35/0.61 |
| E4 | 3.7 | 70 | 16.7 | 0.35/0.62 |
| E5 | 4.2 | 74 | 16.8 | 0.33/0.63 |
| E6 | 4.4 | 63 | 17.0 | 0.33/0.62 |
| E7 | 3.8 | 70 | 16.3 | 0.32/0.64 |
| E8 | 3.6 | 79 | 17.3 | 0.32/0.63 |
| E9 | 3.7 | 73 | 17.8 | 0.33/0.62 |
| E10 | 3.3 | 75 | 18.8 | 0.33/0.63 |
| E11 | 3.2 | 76 | 19.7 | 0.33/0.63 |
| E12 | 3.2 | 73 | 19.9 | 0.33/0.63 |
| E13 | 3.1 | 67 | 19.5 | 0.33/0.63 |
| E14 | 3.4 | 64 | 18.8 | 0.33/0.62 |
| E15 | 3.5 | 64 | 17.9 | 0.33/0.63 |
| E16 | 3.3 | 66 | 18.2 | 0.33/0.62 |

1.-15. (canceled)

16. A compound comprising at least one structure having at least three mutually fused structural elements of the formulae (A), (B) and (C): 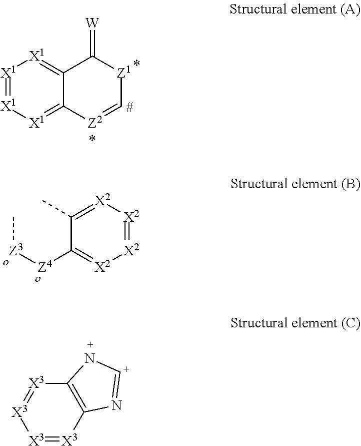
where structural element (A) is fused to structural element (B) and structural element (B) is fused to structural element (C); where structural element (B) binds to structural element (A) via the bonds shown by dotted lines, where there is one bond via the binding site marked # and one bond via a binding site marked *; where structural element (B) is fused to structural element (C) via the atoms marked o and +, and the respectively marked atoms are shared by structural elements (B) and (C), and
the symbols and indices used are as follows:
W is O or S;
Z¹ is NAr, or N if structural element (A) is fused to structural element (B) via Z¹;
Z² is X, or C if structural element (A) is fused to structural element (B) via Z²;
Z³, Z⁴ is N or C, where one of the Z³, Z⁴ radicals is N and one of the Z³, Z⁴ radicals is C;
X is N or CR;
X¹ is the same or different at each instance and is N or CR¹, with the proviso that not more than two of the X¹ groups in one cycle are N;
X² is the same or different at each instance and is N or CR², with the proviso that not more than two of the X² groups in one cycle are N;
X³ is the same or different at each instance and is N or CR³, with the proviso that not more than two of the X³ groups in one cycle are N;
Ar is the same or different at each instance and is an aromatic or heteroaromatic ring system which has 5 to 40 aromatic ring atoms and has R radicals;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for two R radicals or one R radical together with a further radical to form an aliphatic, heteroaliphatic, aromatic or heteroaromatic ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals or one R¹ radical together with a further radical, or an R, R², R³ group, to form an aromatic, heteroaromatic, aliphatic or heteroaliphatic ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical to form an aromatic, heteroaromatic, aliphatic or heteroaliphatic ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical to form an aromatic, heteroaromatic, aliphatic or heteroaliphatic ring system;
Ar′ is the same or different at each instance and is an aromatic or heteroaromatic ring system which has 5 to 40 aromatic ring atoms and has R⁴ radicals;
R⁴ is the same or different at each instance and is H, D, F, Cl, Br, I, R⁵C≡C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system;
R⁵ is the same or different at each instance and is H, D, F or an aliphatic, aromatic or heteroaromatic organic radical, in which one or more hydrogen atoms may also be replaced by F; it is possible here for two or more R⁵ radicals together to form a ring system;
where at least one of the R, R¹, R², R³ radicals is selected from the group consisting of a heteroaromatic ring system which has 6 to 60 aromatic ring atoms and has R⁴ radicals, an aromatic ring system which has 10 to 60 aromatic ring atoms and has R⁴ radicals, an aryloxy group having 10 to 60 aromatic ring atoms or heteroaryloxy group having 6 to 40 aromatic ring atoms, each of which has R⁴ radicals, a diarylamino group having 6 to 60 aromatic ring atoms in the respective aromatic radical, an arylheteroarylamino group having 6 to 60 aromatic ring atoms in the respective aromatic or heteroaromatic radical, and a diheteroarylamino group having 6 to 60 aromatic ring atoms in the respective heteroaromatic radical, where the diarylamino, arylheteroarylamino, diheteroarylamino group has R⁴ radicals.

17. The compound as claimed in claim 16, wherein the compound comprising at least one structure of the formulae (I-1) to (I-7): 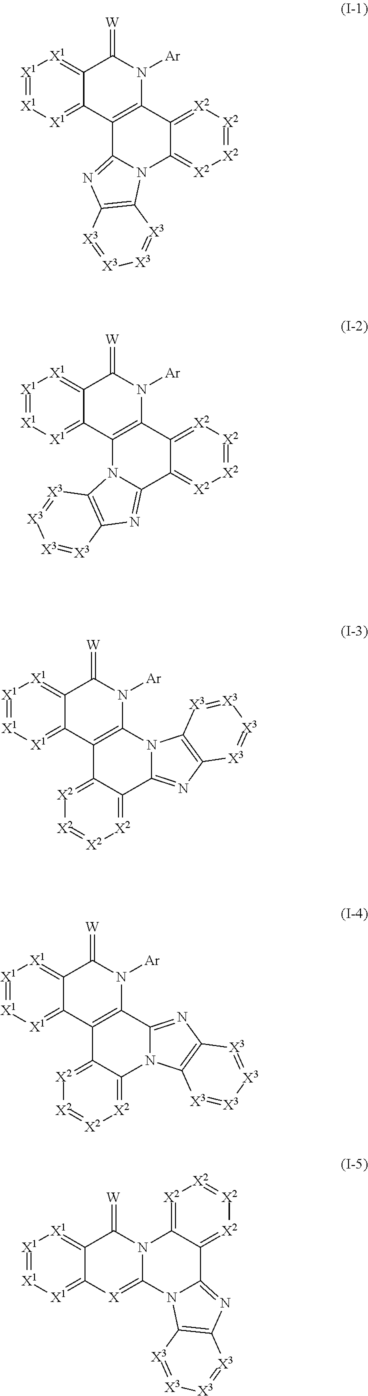 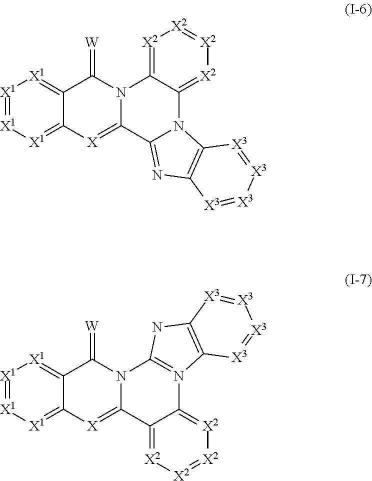
where symbols W, X, X¹, X², X³ and Ar have the definitions given in claim 16.

18. The compound as claimed in claim 16, wherein the compound comprising at least one structure of the formulae (II-1) to (II-30): 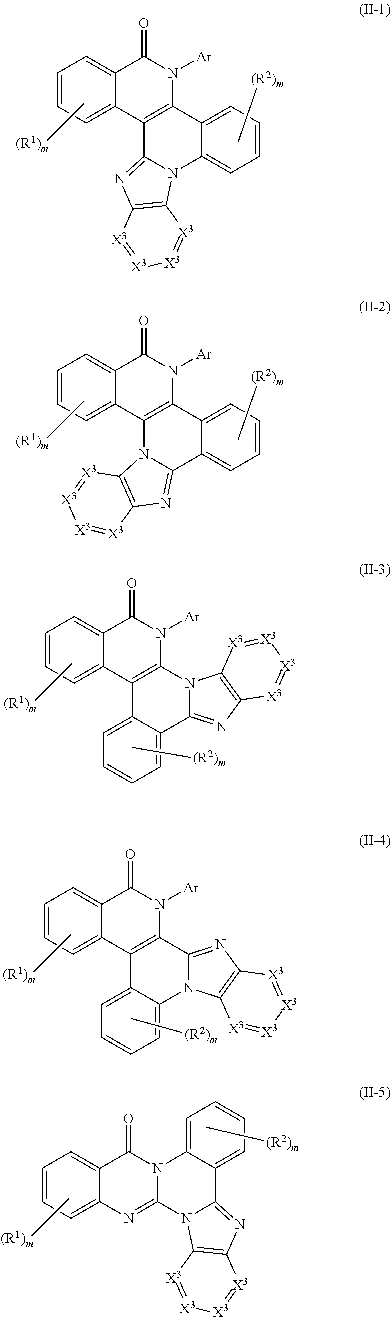 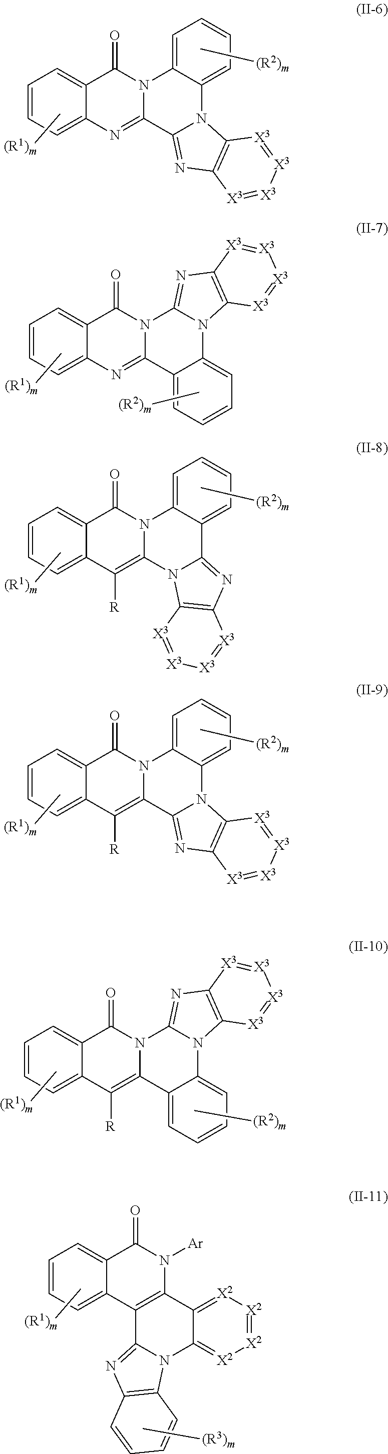 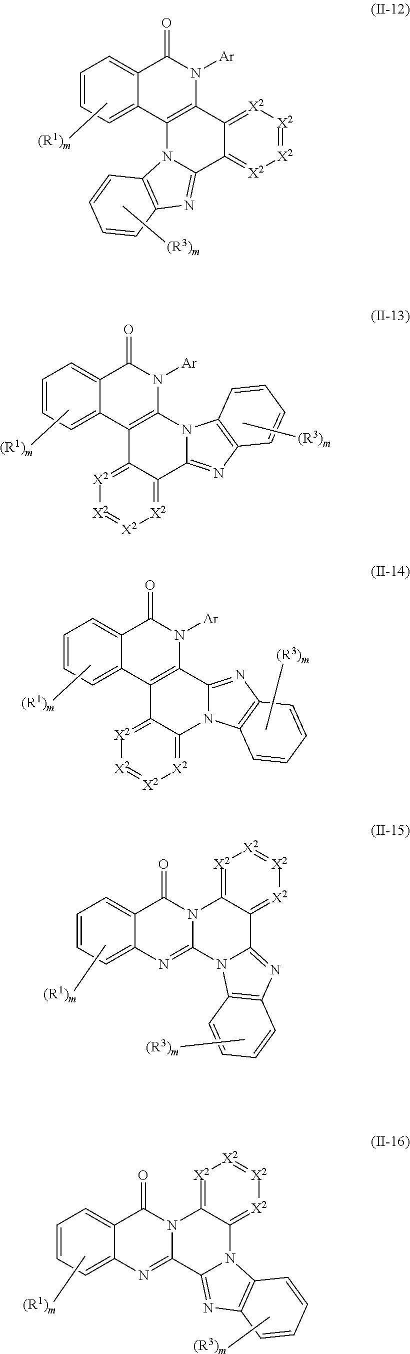 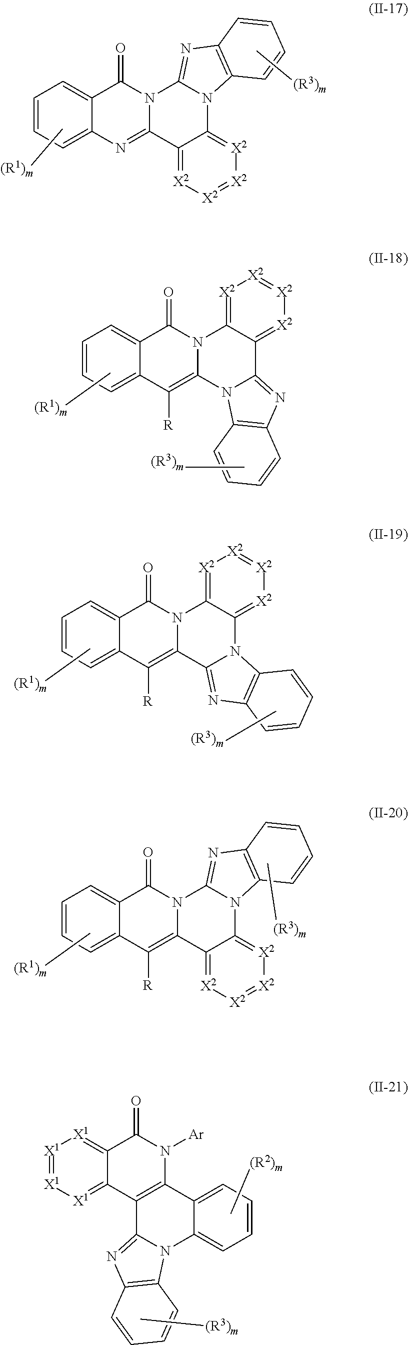 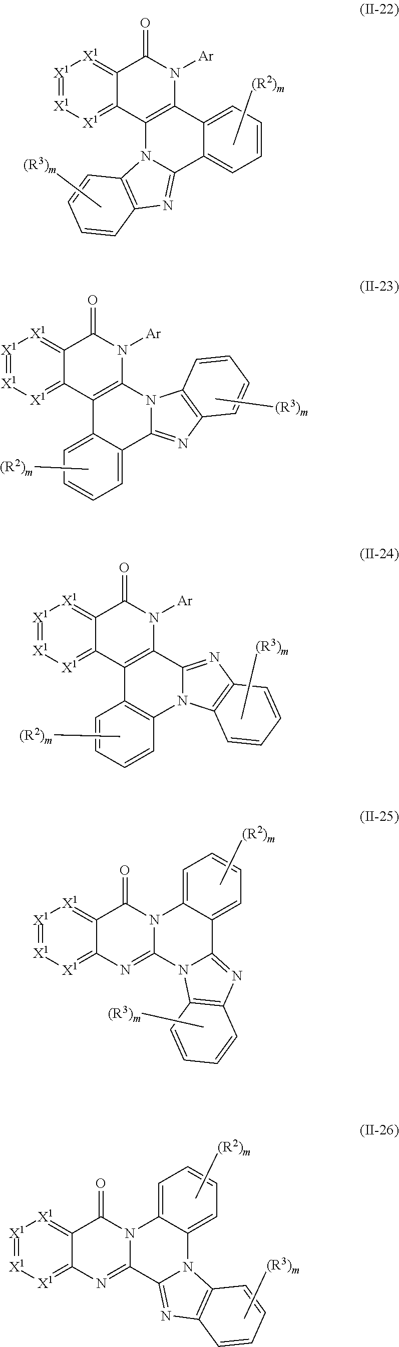 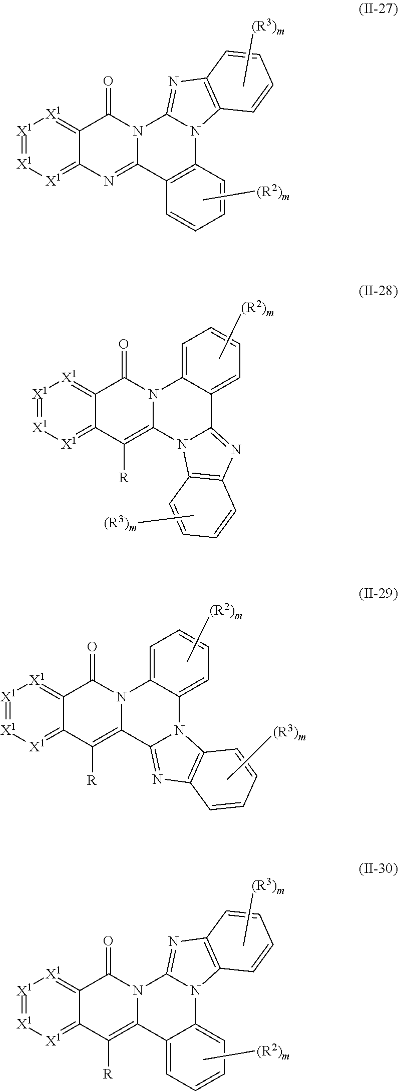
where the symbols X¹, X², X³, R, R¹, R², R³ and Ar have the definitions given in claim 16 and the index m is 0, 1, 2, 3 or 4.

19. The compound as claimed in claim 18, wherein the structure is of the formulae (II-1) to (II-7), (II-11) to (II-17) and/or (II-21) to (II-27).

20. The compound as claimed in claim 16, wherein the compound comprising at least one structure of the formulae (III-1) to (III-10): 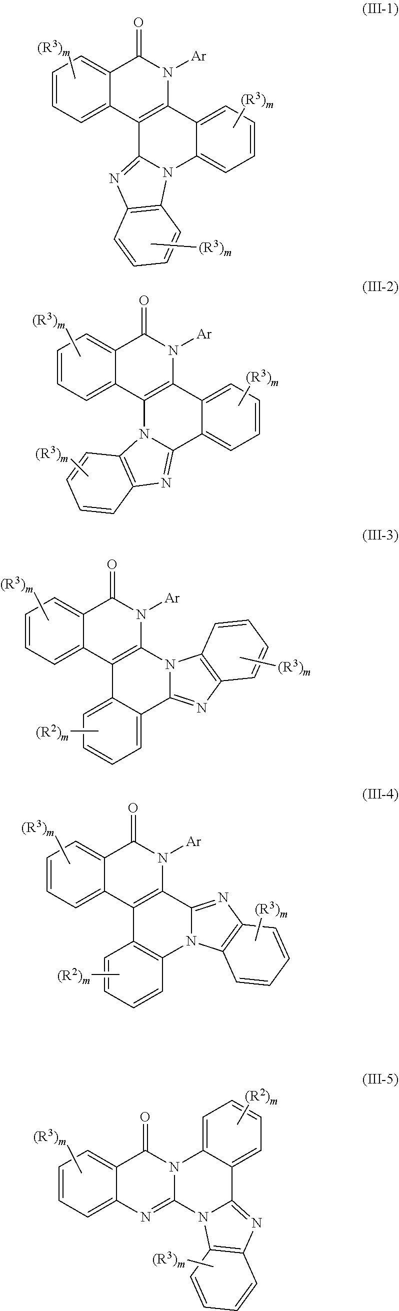 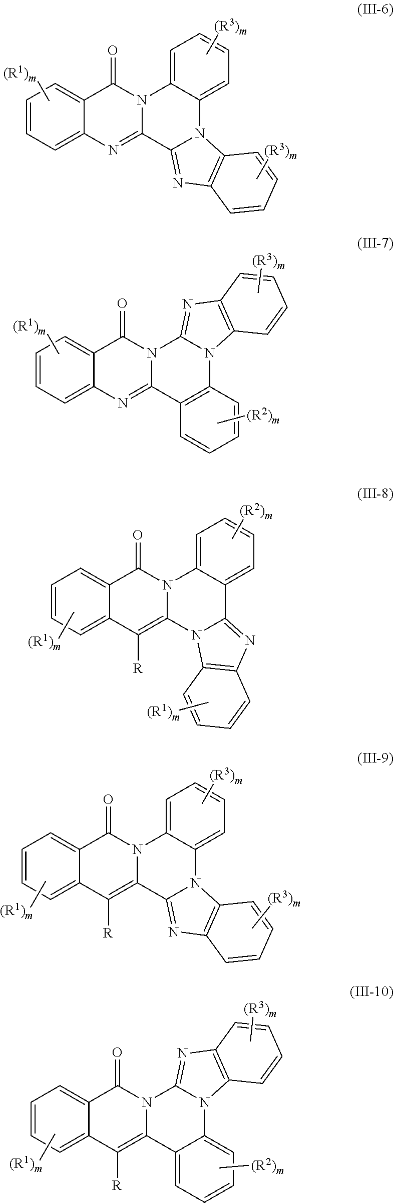
where the symbols R, R¹, R², R³ and Ar have the definitions given in claim 16 and the index m is 0, 1, 2, 3 or 4.

21. The compound as claimed in claim 16, wherein at least one R, R¹, R² and/or R³ group is the same or different and is selected from the radicals of the following formulae SAr-1 to SAr-18: 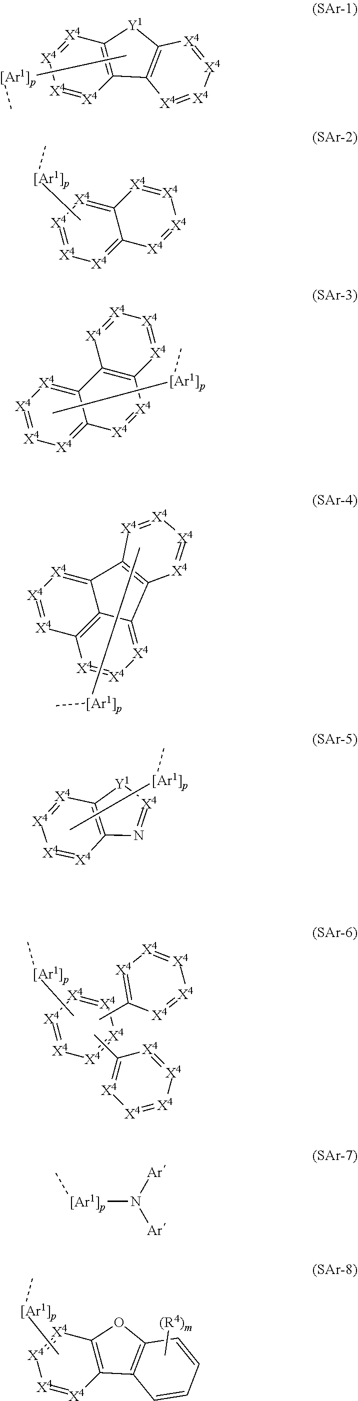 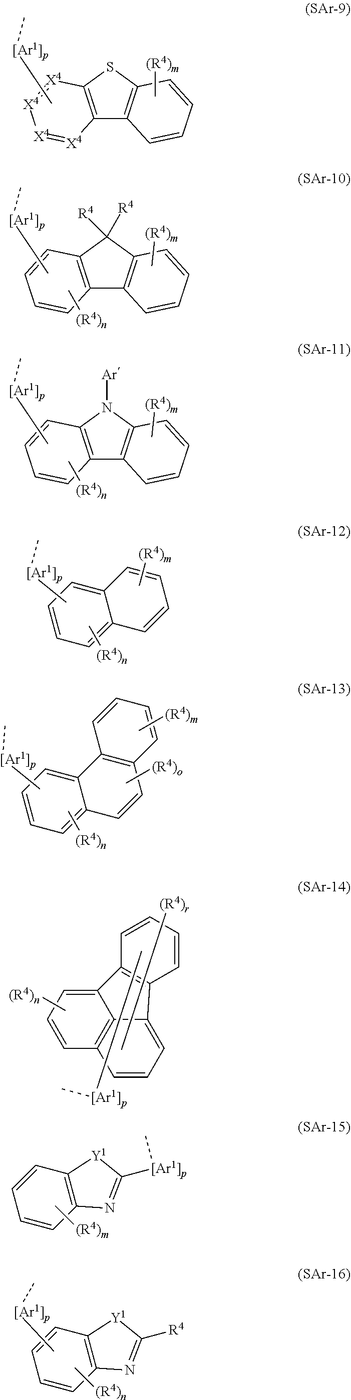 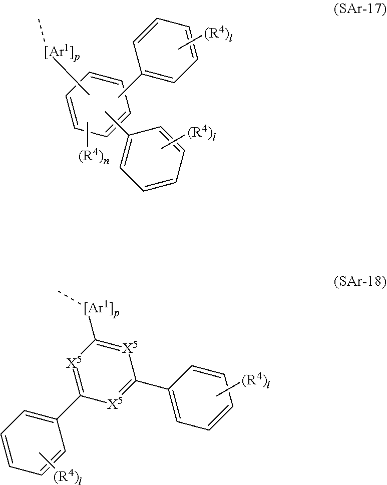
where R⁴ and Ar′ have the definitions given in claim 16 and the further symbols and indices are as follows:
X⁴ is the same or different at each instance and is CR⁴, N, or C if the [Ar¹]ₚ group binds thereto;
X⁵ is the same or different at each instance and is CR⁴ or N;
Ar¹ is the same or different at each instance and is a bivalent aromatic or heteroaromatic ring system which has 6 to 18 aromatic ring atoms and in each case has R⁴ radicals;
Y¹ is the same or different at each instance and is C(R⁴)₂, NR⁴, O, S, or N if the [Ar¹]ₚ group binds thereto;
p is 0 or 1, where p=0 means that the Ar¹ group is absent and that the corresponding aromatic or heteroaromatic group is bonded directly to the corresponding radical;
n is 0, 1, 2 or 3;
m is 0, 1, 2, 3 or 4;
l is 0, 1, 2, 3, 4 or 5; and
r is 0, 1, 2, 3, 4, 5 or 6.

22. The compound as claimed in claim 16, wherein R, R¹, R² and/or R³ are the same or different at each instance and are selected from the group consisting of H, D or an aromatic or heteroaromatic ring system selected from the groups of the following formulae Ar-1 to Ar-79: 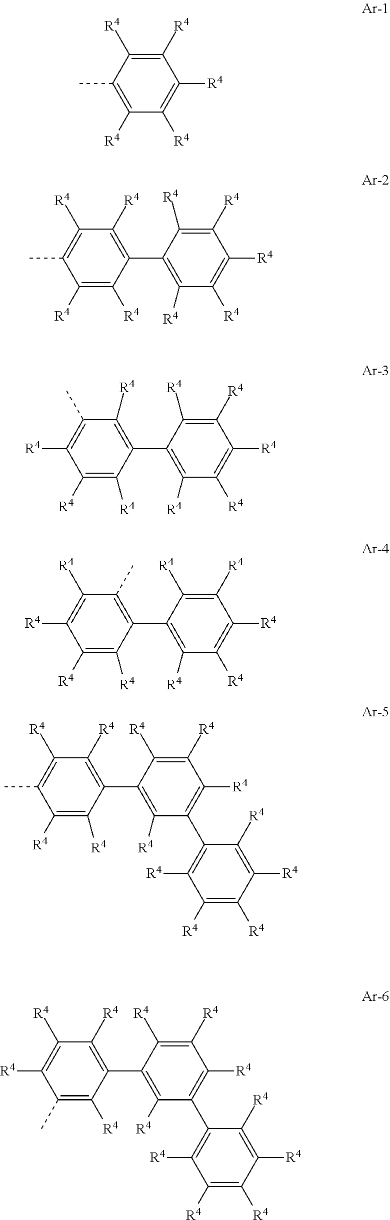 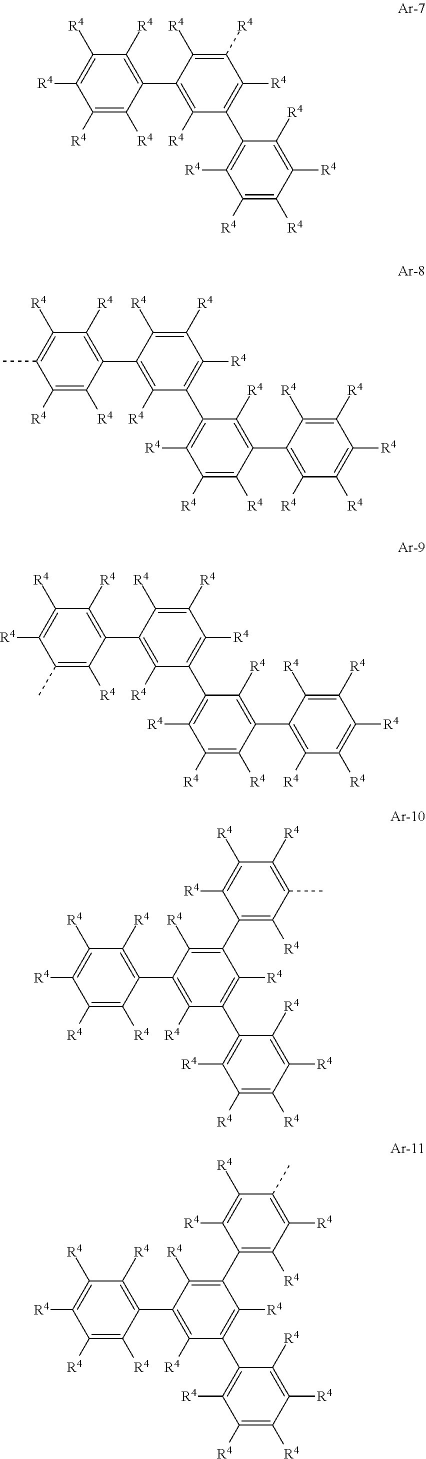 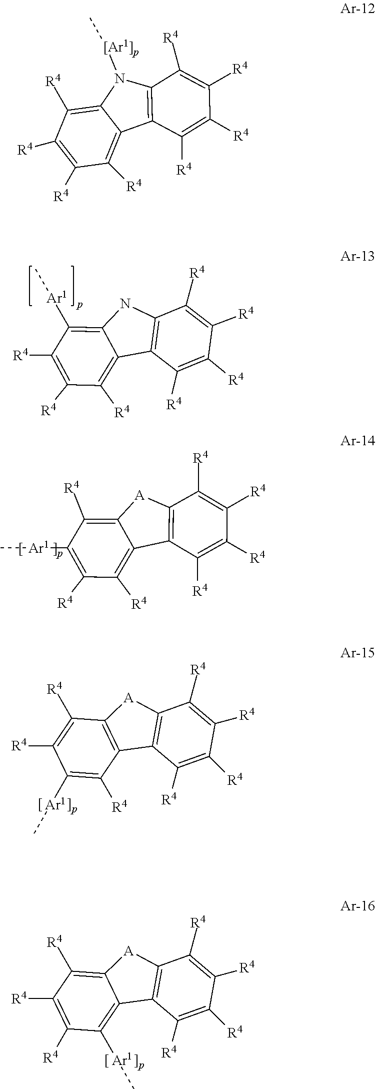 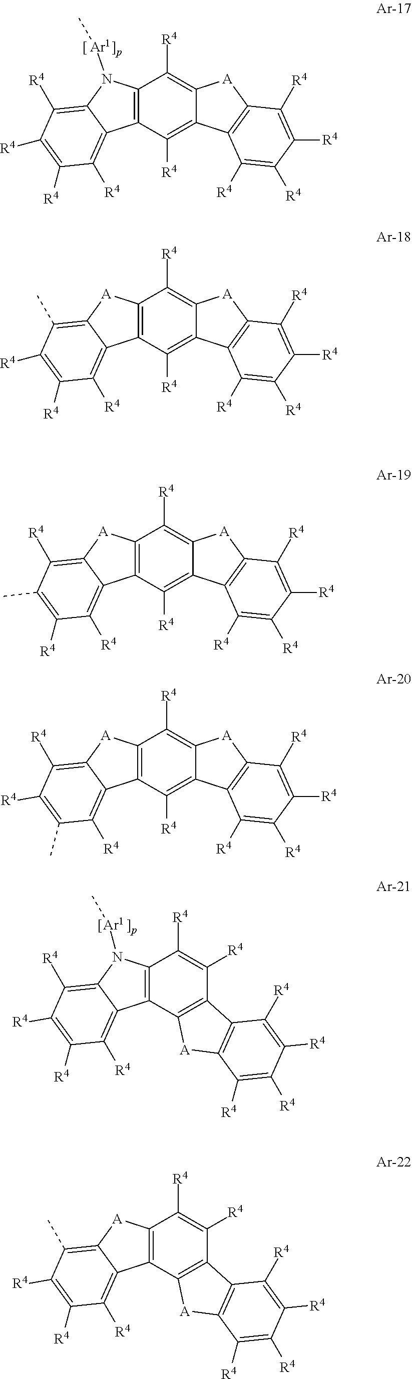 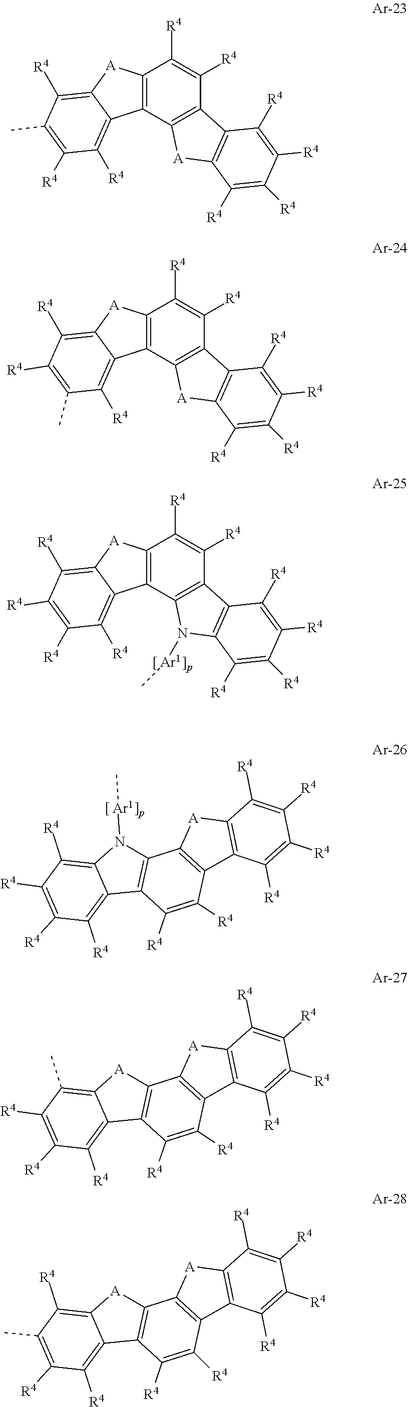 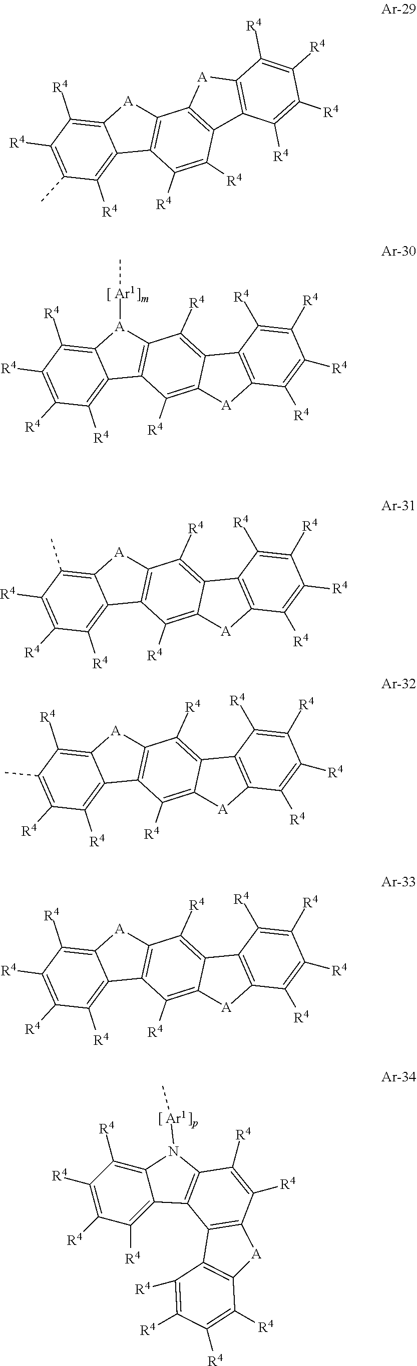 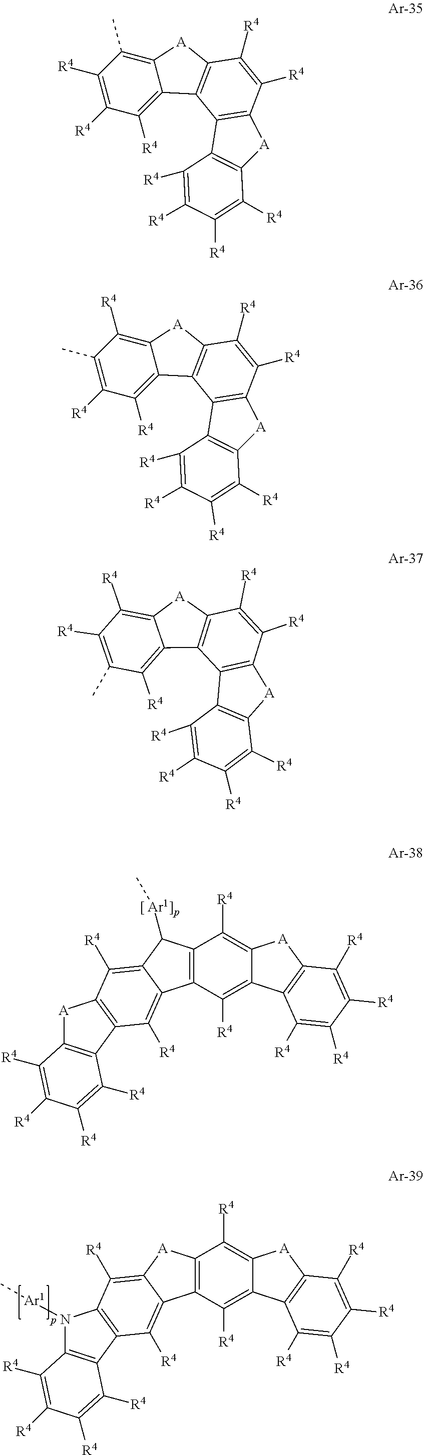 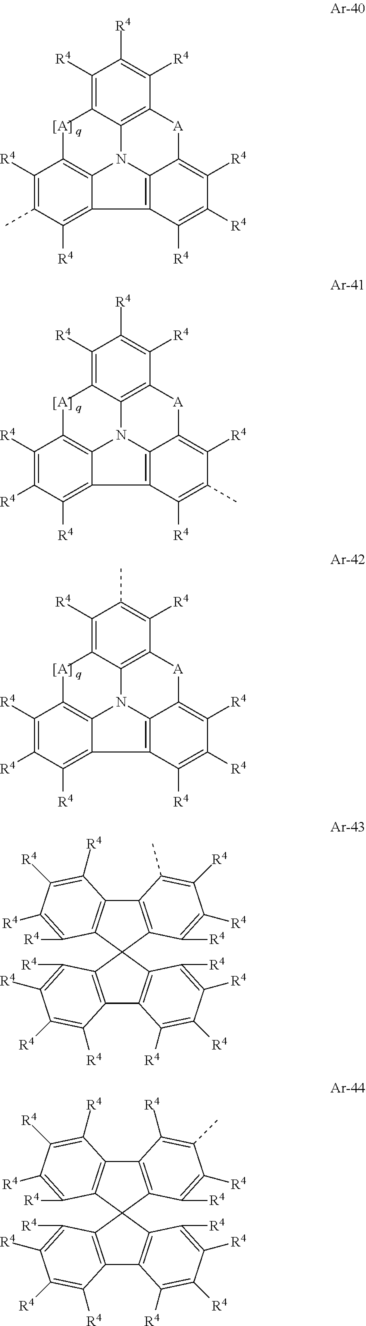 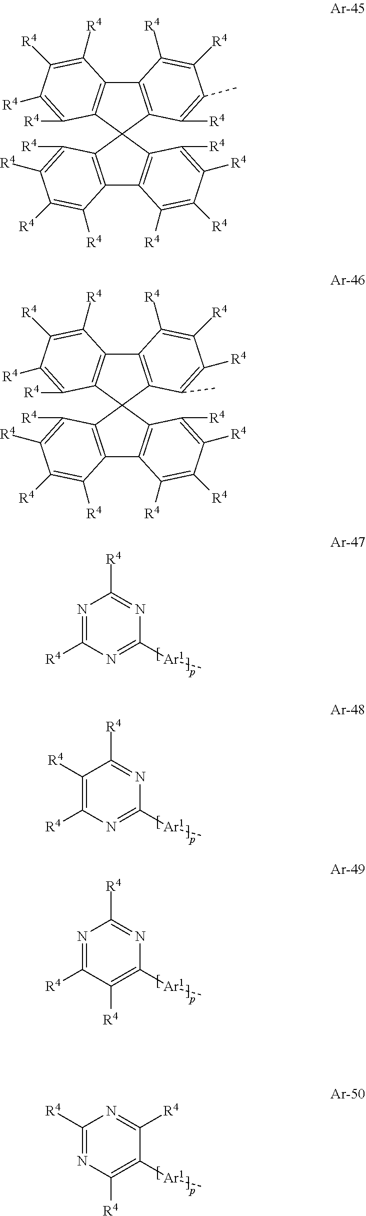 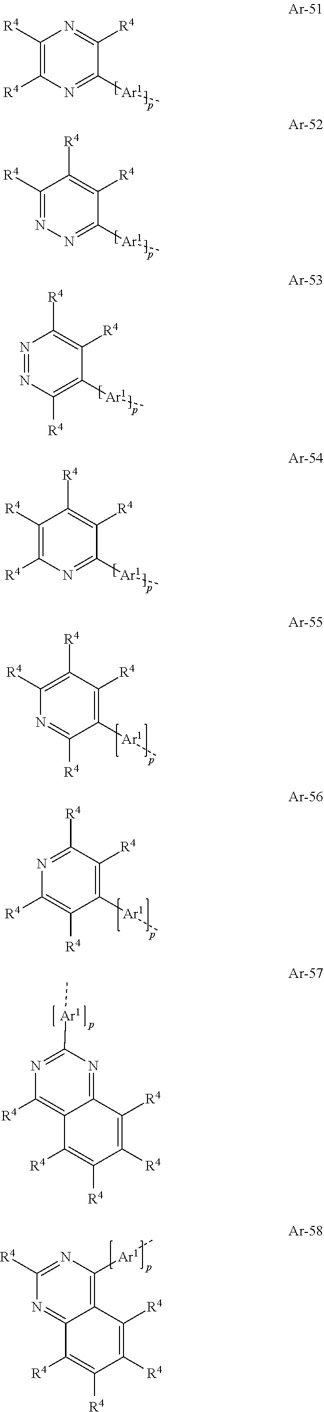 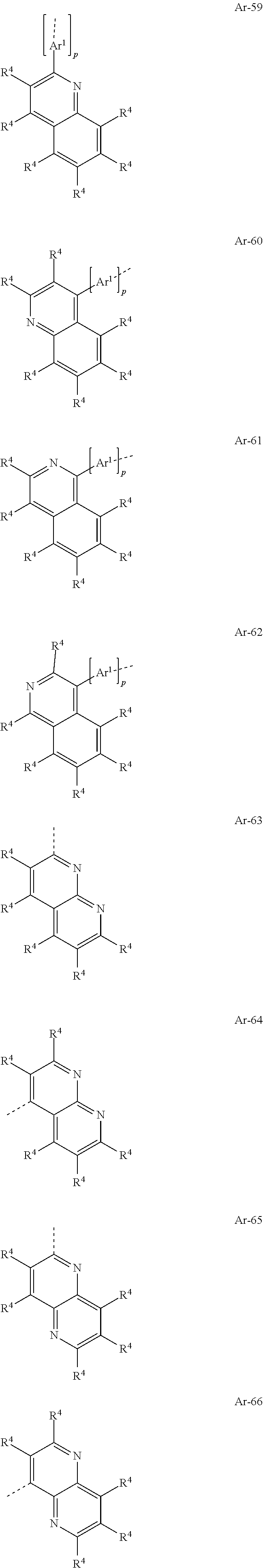 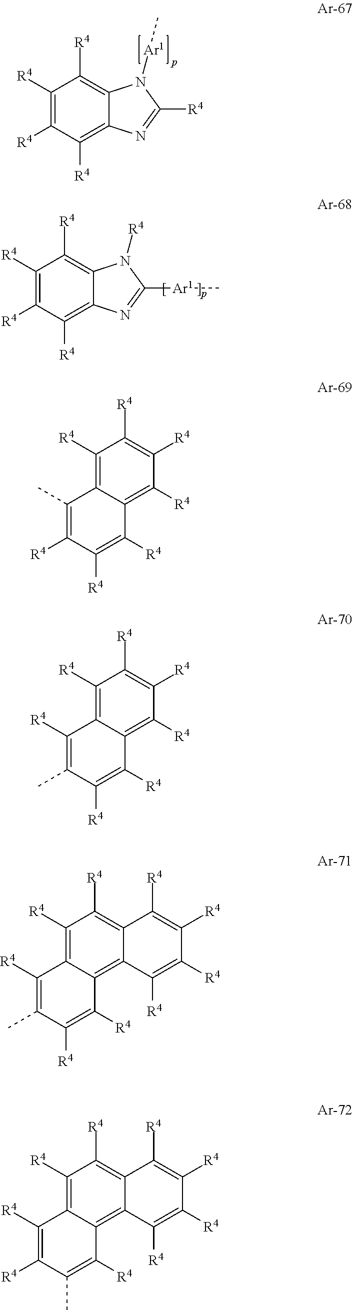 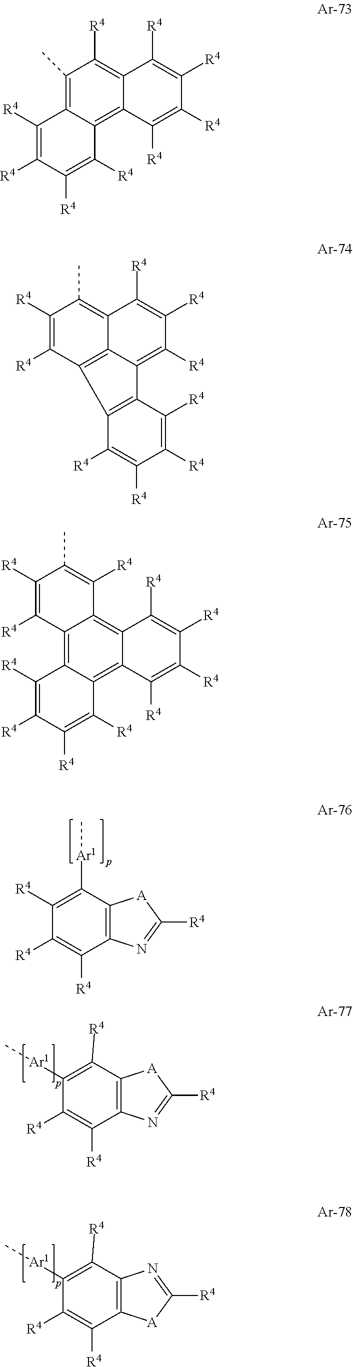 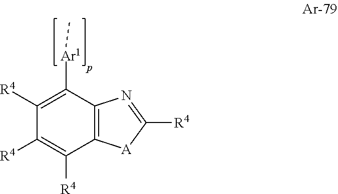
where R⁴ has the definitions given above, the dotted bond represents the bond to the corresponding group and in addition:
Ar¹ is the same or different at each instance and is a bivalent aromatic or heteroaromatic ring system which has 6 to 18 aromatic ring atoms and in each case has R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical; and
q is 0 or 1, where q=0 means that no A group is bonded at this position and R⁴ radicals are bonded to the corresponding carbon atoms instead.

23. A process for preparing the compound as claimed in claim 16, comprising reacting a nitrogen-containing aromatic or heteroaromatic compound in a ring-forming reaction.

24. A composition comprising at least one compound comprising at least one structure having at least three mutually fused structural elements of the formulae (A′), (B′) and (C′): 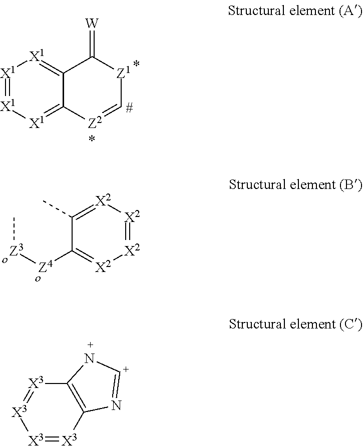
where structural element (A′) is fused to structural element (B′) and structural element (B′) is fused to structural element (C′); where structural element (B′) binds to structural element (A′) via the bonds shown by dotted lines, where there is one bond via the binding site marked # and one bond via a binding site marked *; where structural element (B′) is fused to structural element (C′) via the atoms marked o and +, and the respectively marked atoms are shared by structural elements (B′) and (C′), and
the symbols and indices used are as follows:
W is O or S;
Z¹ is NAr, or N if structural element (A′) is fused to structural element (B′) via Z¹;
Z² is X, or C if structural element (A′) is fused to structural element (B′) via Z²;
Z³, Z⁴ is N or C, where one of the Z³, Z⁴ radicals is N and one of the Z³, Z⁴ radicals is C;
X is N or CR;
X¹ is the same or different at each instance and is N or CR¹, with the proviso that not more than two of the X¹ groups in one cycle are N;
X² is the same or different at each instance and is N or CR², with the proviso that not more than two of the X² groups in one cycle are N;
X³ is the same or different at each instance and is N or CR³, with the proviso that not more than two of the X³ groups in one cycle are N;
Ar is the same or different at each instance and is an aromatic or heteroaromatic ring system which has 5 to 40 aromatic ring atoms and has R radicals;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for one R radical together with a further radical to form an aliphatic, heteroaliphatic, aromatic or heteroaromatic ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals or one R¹ radical together with a further radical to form an aliphatic, heteroaliphatic, aromatic or heteroaromatic ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical to form an aliphatic, heteroaliphatic, aromatic or heteroaromatic ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical;
Ar′ is the same or different at each instance and is an aromatic or heteroaromatic ring system which has 5 to 40 aromatic ring atoms and has R⁴ radicals;
R⁴ is the same or different at each instance and is H, D, F, Cl, Br, I, R⁵C≡C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system;
R⁵ is the same or different at each instance and is H, D, F or an aliphatic, aromatic or heteroaromatic organic radical, having 1 to 20 carbon atoms, in which one or more hydrogen atoms may also be replaced by F; it is possible here for two or more R⁵ radicals together to form a ring system;
and
at least one further matrix material, where the further matrix material is selected from compounds of at least one of the formulae (H-1), (H-2), (H-3), (H-4) and (H-5): 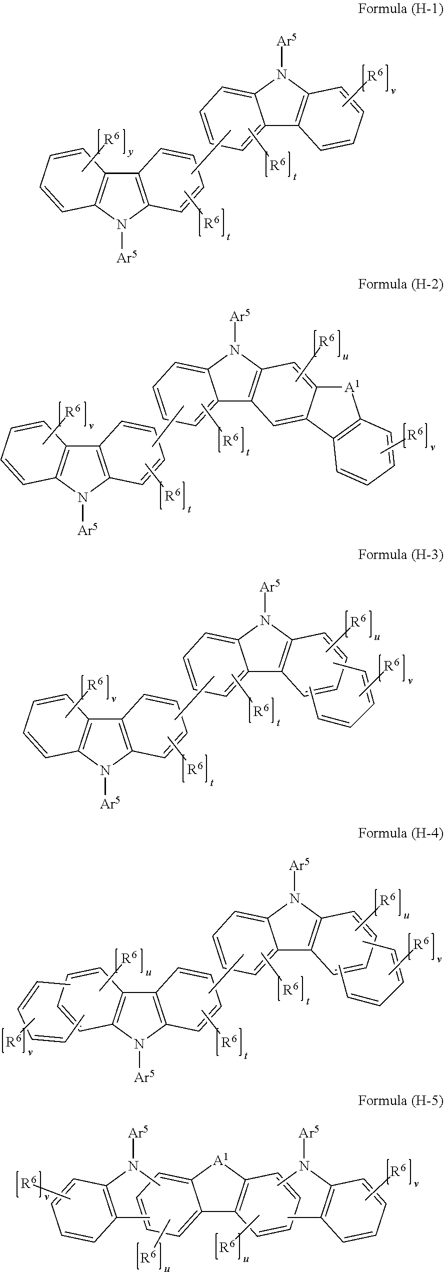
where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and in each case has R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and has R⁷ radicals;
A¹ is C(R⁷)₂, NR⁷, O or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R′)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3; and
u is the same or different at each instance and is 0, 1 or 2.

25. The composition as claimed in claim 24, wherein the composition consists of at least one compound containing at least one compound comprising at least one structure having at least three mutually fused structural elements of the formulae (A′), (B′) and (C′), and at least one compound of one of the formulae (H-1), (H-2), (H-3), (H-4) and (H-5).

26. The composition as claimed in claim 24, wherein the compounds of one of the formulae (H-1), (H-2), (H-3), (H-4) and (H-5) have a proportion by mass in the composition in the range from 5% by weight to 90% by weight, based on the overall composition.

27. The composition as claimed in claim 24, wherein the compounds of one of the formulae (H-1), (H-2), (H-3), (H-4) and (H-5) have a proportion by mass in the composition in the range from 30% by weight to 50% by weight, based on the overall composition.

28. A formulation comprising at least one compound as claimed in claim 16 and at least one further compound.

29. A formulation comprising at least one composition as claimed in claim 24 and at least one further compound.

30. A formulation comprising at least one composition as claimed in claim 24 and at least one or more solvents.

31. A formulation comprising at least one compound as claimed in claim 16 and at least one or more solvents.

32. An electronic device containing at least one compound comprising at least one structure having at least three mutually fused structural elements of the formulae (A′), (B′) and (C′): 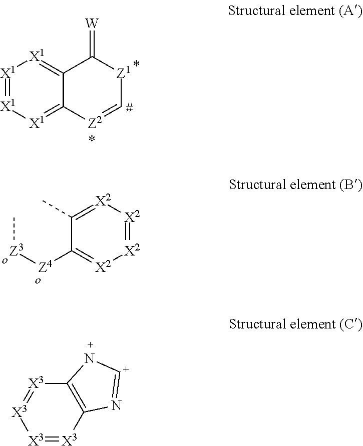
where structural element (A′) is fused to structural element (B′) and structural element (B′) is fused to structural element (C′); where structural element (B′) binds to structural element (A′) via the bonds shown by dotted lines, where there is one bond via the binding site marked # and one bond via a binding site marked *; where structural element (B′) is fused to structural element (C′) via the atoms marked o and +, and the respectively marked atoms are shared by structural elements (B′) and (C′), wherein
W is O or S;
Z¹ is NAr, or N if structural element (A′) is fused to structural element (B′) via Z¹;
Z² is X, or C if structural element (A′) is fused to structural element (B′) via Z²;
Z³, Z⁴ is N or C, where one of the Z³, Z⁴ radicals is N and one of the Z³, Z⁴ radicals is C;
X is N or CR;
X¹ is the same or different at each instance and is N or CR¹, with the proviso that not more than two of the X¹ groups in one cycle are N;
X² is the same or different at each instance and is N or CR², with the proviso that not more than two of the X² groups in one cycle are N;
X³ is the same or different at each instance and is N or CR³, with the proviso that not more than two of the X³ groups in one cycle are N;
Ar is the same or different at each instance and is an aromatic or heteroaromatic ring system which has 5 to 40 aromatic ring atoms and has R radicals;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for one R radical together with a further radical to form an aliphatic, heteroaliphatic, aromatic or heteroaromatic ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals or one R¹ radical together with a further radical to form an aliphatic, heteroaliphatic, aromatic or heteroaromatic ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical to form an aliphatic, heteroaliphatic, aromatic or heteroaromatic ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical;
Ar′ is the same or different at each instance and is an aromatic or heteroaromatic ring system which has 5 to 40 aromatic ring atoms and has R⁴ radicals;
R⁴ is the same or different at each instance and is H, D, F, Cl, Br, I, R⁵C≡C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system;
R⁵ is the same or different at each instance and is H, D, F or an aliphatic, aromatic or heteroaromatic organic radical, having 1 to 20 carbon atoms, in which one or more hydrogen atoms may also be replaced by F; it is possible here for two or more R⁵ radicals together to form a ring system

33. The electronic device as claimed in claim 32, wherein the device is an organic electroluminescent device.

34. The electronic device as claimed in claim 32 which is an organic electroluminescent device, wherein the compound comprising at least one structure having at least three mutually fused structural elements of the formulae (A′), (B′) and (C′) is used as matrix material in an emitting layer and/or in an electron transport layer and/or in a hole blocker layer.

35. The electronic device as claimed in claim 32, wherein the compound comprising at least one structure having at least three mutually fused structural elements of the formulae (A′), (B′) and (C′), is used as matrix material for phosphorescent emitters in combination with a further matrix material selected from compounds of one of the formulae (H-1), (H-2), (H-3), (H-4) and (H-5), 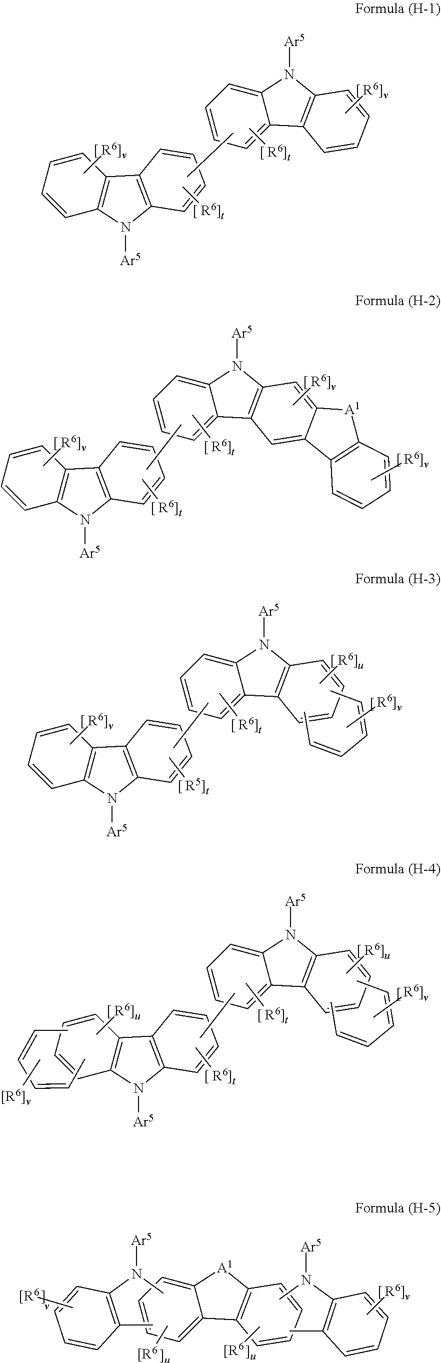
where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and in each case has R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and has R⁷ radicals;
A¹ is C(R⁷)₂, NR⁷, 0 or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R′)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3; and
u is the same or different at each instance and is 0, 1 or 2.
